(19) United States
(12) Patent Application Publication
(10) Pub. No.: US 2025/0206699 A1
(43) Pub. Date: Jun. 26, 2025

(54) LIPID COMPOUNDS AND LIPID NANOPARTICLES

(71) Applicant: BroadPharm Inc., San Diego, CA (US)

(72) Inventors: Hangzhong Zhang, San Diego, CA (US); Irawan Pramudya, San Diego, CA (US); Mikhail A. Kondratenko, San Diego, CA (US)

(73) Assignee: BroadPharm Inc., San Diego, CA (US)

(21) Appl. No.: 18/847,232

(22) PCT Filed: Mar. 14, 2023
(86) PCT No.: PCT/US2023/064282

§ 371 Date: Sep. 14, 2024

Related U.S. Application Data
(60) Provisional application No. 63/364,020, filed on May 2, 2022.
(60) Provisional application No. 63/269,397, filed on Mar. 15, 2022.

(51) Int. Cl. C07C 233/36; C07C 217/08; C07C 219/06; C07C 219/08; C07C 219/16; C07C 229/16; C07C 271/12; C07C 271/20

(52) U.S. Cl. CPC C07C 233/36; C07C 217/08; C07C 219/06; C07C 219/08; C07C 219/16; C07C 229/16; C07C 271/12; C07C 271/20

(57) ABSTRACT

Provided herein are lipid compounds, e.g., a compound of Formula (I). Also provided are lipid nanoparticles, each comprising a lipid compound of Formula (I).

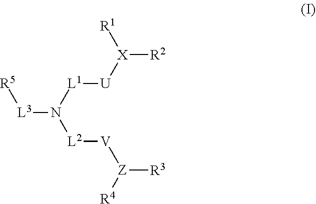

### CROSS REFERENCE TO RELATED APPLICATIONS

This application claims the benefit of the priority of U.S. Provisional Application Nos. 63/269,397, filed Mar. 25, 2022, and 63/364,020, filed May 2, 2022; the disclosure of each of which is incorporated herein by reference in its entirety.

### FIELD

Provided herein are lipid compounds. Also provided are lipid nanoparticles, each comprising a lipid compound.

### BACKGROUND

Lipid nanoparticles (LNPs) are a promising delivery vehicle for a variety of therapeutic agents, including small molecule drugs, proteins, and nucleic acids. Akinc et al., Nat. Nanotechnol. 2019, 14, 1084-87; Hou et al., Nat. Rev. Mater. 2021, 6, 1078-94. The two FDA-approved COVID-19 mRNA vaccines, mRNA-1273 and BNT162b, employ lipid nanoparticles for antigen mRNA delivery. Polack et al., N. Engl. J. Med 2020, 383, 2603-15; Baden et al., N. Engl. J. Med. 2021, 384, 403-16. However, it remains challenging to deliver a therapeutic agent efficiently, especially a highly charged nucleic acid, such as an mRNA. Weng et al., Biotechnol. Adv. 2020, 40, 107534; Hou et al., Nat. Rev. Mater. 2021, 6, 1078-94. To achieve therapeutic effects, for example, a highly charged mRNA molecules have to reach specific target cells and produce sufficient proteins of interest. Hou et al., Nat. Rev. Mater. 2021, 6, 1078-94. Therefore, there is a need for a lipid compound for delivering a therapeutic agent efficiently.

### SUMMARY OF THE DISCLOSURE

Provided herein is a compound of Formula (I):

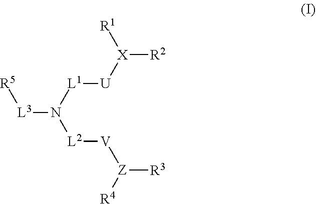

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, or hydrate thereof; wherein:
R¹ is hydrogen, C₁₋₂₀ alkyl, C₁₋₂₀ heteroalkyl, C₂₋₂₀ alkenyl, or C₂₋₂₀ alkynyl;
R², R³, and R⁴ are each independently C₁₋₂₀ alkyl, C₁₋₂₀ heteroalkyl, C₂₋₂₀ alkenyl, or C₂₋₂₀ alkynyl;
R⁵ is —C(O)OR^{5a}, —OR^{5a}, or —OC(O)R^{5a}; wherein each R^{5a} is independently hydrogen, C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl;
L¹, L², and L³ are each independently C₁₋₂₀ alkylene, C₁₋₂₀ heteroalkylene, C₂₋₂₀ alkenylene, or C₂₋₂₀ alkynylene;
U and V are each independently —C(O)—, —C(O)O—, or —OC(O)O—; and
X and Y are each independently C(H) or N;
with the proviso that, when U and V are each —C(O)O—, and X and Z are each C(H), then at least one of R¹, R², R³, and R⁴ is substituted C₁₋₂₀ alkyl, or at least one of L¹ and L² is C₁₋₂₀ heteroalkylene;
wherein each alkyl, alkylene, heteroalkyl, heteroalkylene, alkenyl, alkenylene, alkynyl, alkynylene, cycloalkyl, aryl, aralkyl, heteroaryl, and heterocyclyl is optionally substituted with one or more, in one embodiment, one, two, three, or four, substituents Q, wherein each Q is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{b})OR^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, nitro, imino, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OP(O)(OR^{f})OR^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

Additionally, provided herein is a lipid nanoparticle comprising a compound of Formula (I), or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, or hydrate thereof.

### DETAILED DESCRIPTION

To facilitate understanding of the disclosure set forth herein, a number of terms are defined below.

Generally, the nomenclature used herein and the laboratory procedures in organic chemistry, medicinal chemistry, and biochemistry described herein are those well-known and commonly employed in the art. Unless defined otherwise, all technical and scientific terms used herein generally have the same meaning as commonly understood by one of ordinary skill in the art to which this disclosure belongs.

The term “alkyl” refers to a linear or branched saturated monovalent hydrocarbon radical, wherein the alkyl is optionally substituted with one or more substituents Q as described herein. For example, C₁₋₆ alkyl refers to a linear saturated monovalent hydrocarbon radical of 1 to 6 carbon atoms or a branched saturated monovalent hydrocarbon radical of 3 to 6 carbon atoms. In certain embodiments, the alkyl is a linear saturated monovalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ alkyl groups are also referred as “lower alkyl.” Examples of alkyl groups include, but are not limited to, methyl, ethyl, propyl (including all isomeric forms, e.g., n-propyl and isopropyl), butyl (including all isomeric forms, e.g., n-butyl, isobutyl, sec-butyl, and t-butyl), pentyl (including all isomeric forms, e.g., n-pentyl, isopentyl, sec-pentyl, neopentyl, and tert-pentyl), and hexyl (including all isomeric forms, e.g., n-hexyl, isohexyl, and sec-hexyl).

The terms “alkylene” and “alkanediyl” are used interchangeably herein in reference to a linear or branched saturated divalent hydrocarbon radical, wherein the alkanediyl is optionally be substituted with one or more substituents Q as described herein. For example, C₁₋₆ alkanediyl refers to a linear saturated divalent hydrocarbon radical of 1 to 6 carbon atoms or a branched saturated divalent hydrocarbon radical of 3 to 6 carbon atoms. In certain embodiments, the alkanediyl is a linear saturated divalent hydrocarbon radical that has 1 to 30 (C₁₋₃₀), 1 to 20 (C₁₋₂₀), 1 to 15 (C₁₋₁₅), 1 to 10 (C₁₋₁₀), or 1 to 6 (C₁₋₆) carbon atoms, or branched saturated divalent hydrocarbon radical of 3 to 30 (C₃₋₃₀), 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ alkanediyl groups are also referred as “lower alkanediyl.” Examples of alkanediyl groups include, but are not limited to, methanediyl, ethanediyl (including all isomeric forms, e.g., ethane-1,1-diyl and ethane-1,2-diyl), propanediyl (including all isomeric forms, e.g., propane-1,1-diyl, propane-1,2-diyl, and propane-1,3-diyl), butanediyl (including all isomeric forms, e.g., butane-1,1-diyl, butane-1,2-diyl, butane-1,3-diyl, and butane-1,4-diyl), pentanediyl (including all isomeric forms, e.g., pentane-1,1-diyl, pentane-1,2-diyl, pentane-1,3-diyl, and pentane-1,5-diyl), and hexanediyl (including all isomeric forms, e.g., hexane-1,1-diyl, hexane-1,2-diyl, hexane-1,3-diyl, and hexane-1,6-diyl). Examples of substituted alkanediyl groups include, but are not limited to, —C(O)CH₂—, —C(O)(CH₂)₂—, —C(O)(CH₂)₃—, —C(O)(CH₂)₄—, —C(O)(CH₂)₅—, —C(O)(CH₂)₆—, —C(O)(CH₂)₇—, —C(O)(CH₂)₈—, —C(O)(CH₂)₉—, —C(O)(CH₂)₁₀—, —C(O)CH₂C(O)—, —C(O)(CH₂)₂C(O)—, —C(O)(CH₂)₃C(O)—, —C(O)(CH₂)₄C(O)—, or —C(O)(CH₂)₅C(O)—.

The term “heteroalkyl” refers to a linear or branched saturated monovalent hydrocarbon radical that contains one or more heteroatoms on its main chain, each independently selected from O, S, and N. The heteroalkyl is optionally substituted with one or more substituents Q as described herein. For example, C₁₋₆ heteroalkyl refers to a linear saturated monovalent hydrocarbon radical of 1 to 6 carbon atoms or a branched saturated monovalent hydrocarbon radical of 3 to 6 carbon atoms. In certain embodiments, the heteroalkyl is a linear saturated monovalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ heteroalkyl groups are also referred as “lower heteroalkyl.” Examples of heteroalkyl groups include, but are not limited to, —OCH₃, —OCH₂CH₃, —CH₂OCH₃, —NHCH₃, —ONHCH₃, —NHOCH₃, —SCH₃, —CH₂NHCH₂CH₃, and —NHCH₂CH₂CH₃. Examples of substituted heteroalkyl groups include, but are not limited to, —CH₂NHC(O)CH₃ and —NHC(O)CH₂CH₃.

The terms “heteroalkylene” and “heteroalkanediyl” are used interchangeably herein in reference to a linear or branched saturated divalent hydrocarbon radical that contains one or more heteroatoms in its main chain, each independently selected from O, S, and N. The heteroalkylene is optionally substituted with one or more substituents Q as described herein. For example, C₁₋₆ heteroalkylene refers to a linear saturated divalent hydrocarbon radical of 1 to 6 carbon atoms or a branched saturated divalent hydrocarbon radical of 3 to 6 carbon atoms. In certain embodiments, the heteroalkylene is a linear saturated divalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated divalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ heteroalkylene groups are also referred as “lower heteroalkylene.” Examples of heteroalkylene groups include, but are not limited to, —CH₂O—, —(CH₂)₂O—, —(CH₂)₃O—, —(CH₂)₄O—, —(CH₂)₅O—, —(CH₂)₆O—, —(CH₂)₇O—, —(CH₂)₈O—, —(CH₂)₉O—, —(CH₂)₁₀O—, —CH₂OCH₂—, —CH₂CH₂O—, —(CH₂CH₂O)₂—, —(CH₂CH₂O)₃—, —(CH₂CH₂O)₄—, —(CH₂CH₂O)₅—, —CH₂NH—, —CH₂NHCH₂—, —CH₂CH₂NH—, —CH₂S—, —CH₂SCH₂—, and —CH₂CH₂S—. Examples of substituted heteroalkylene groups include, but are not limited to, —C(O)CH₂O—, —C(O)(CH₂)₂O—, —C(O)(CH₂)₃O—, —C(O)(CH₂)₄O—, —C(O)(CH₂)₅O—, —C(O)(CH₂)₆O—, —C(O)(CH₂)₇O—, —C(O)(CH₂)₈O—, —C(O)(CH₂)₉O—, —C(O)(CH₂)₁₀O—, —C(O)CH₂OCH₂CH₂O—, —C(O)CH₂O(CH₂CH₂O)₂—, —C(O)CH₂O(CH₂CH₂O)₃—, —C(O)CH₂O(CH₂CH₂O)₄, —C(O)CH₂O(CH₂CH₂O)₅—, —CH₂NHC(O)CH₂—, or —CH₂CH₂C(O)NH—.

The term “alkenyl” refers to a linear or branched monovalent hydrocarbon radical, which contains one or more, in one embodiment, one, two, three, or four, in another embodiment, one, carbon-carbon double bond(s). The alkenyl is optionally substituted with one or more substituents Q as described herein. The term “alkenyl” embraces radicals having a “cis” or “trans” configuration or a mixture thereof, or alternatively, a “Z” or “E” configuration or a mixture thereof, as appreciated by those of ordinary skill in the art. For example, C₂₋₆ alkenyl refers to a linear unsaturated monovalent hydrocarbon radical of 2 to 6 carbon atoms or a branched unsaturated monovalent hydrocarbon radical of 3 to 6 carbon atoms. In certain embodiments, the alkenyl is a linear monovalent hydrocarbon radical of 2 to 20 (C₂₋₂₀), 2 to 15 (C₂₋₄₅), 2 to 10 (C₂₋₁₀), or 2 to 6 (C₂₋₆) carbon atoms, or a branched monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkenyl groups include, but are not limited to, ethenyl, propenyl (including all isomeric forms, e.g., propen-1-yl, propen-2-yl, and allyl), and butenyl (including all isomeric forms, e.g., buten-1-yl, buten-2-yl, buten-3-yl, and 2-buten-1-yl).

The terms “alkenylene” and “alkenediyl” are used interchangeably herein in reference to a linear or branched divalent hydrocarbon radical, which contains one or more, in one embodiment, one, two, three, or four, in another embodiment, one, carbon-carbon double bond(s). The alkenediyl is optionally substituted with one or more substituents Q as described herein. The term “alkenediyl” embraces radicals having a “cis” or “trans” configuration or a mixture thereof, or alternatively, a “Z” or “F” configuration or a mixture thereof, as appreciated by those of ordinary skill in the art. For example, C₂₋₆ alkenediyl refers to a linear unsaturated divalent hydrocarbon radical of 2 to 6 carbon atoms or a branched unsaturated divalent hydrocarbon radical of 3 to 6 carbon atoms. In certain embodiments, the alkenediyl is a linear divalent hydrocarbon radical of 2 to 30 (C₂₋₃₀), 2 to 20 (C₂₋₂₀), 2 to 15 (C₂₋₁₅), 2 to 10 (C₂₋₁₀), or 2 to 6 (C₂₋₆) carbon atoms, or a branched divalent hydrocarbon radical of 3 to 30 (C₃₋₃₀), 3 to 20 (C₃₋₂₀), 3 to 15 (C₃₋₁₅), 3 to 10 (C₃₋₁₀), or 3 to 6 (C₃₋₆) carbon atoms. Examples of alkenediyl groups include, but are not limited to, ethenediyl (including all isomeric forms, e.g., ethene-1,1-diyl and ethene-1,2-diyl), propenediyl (including all isomeric forms, e.g., 1-propene-1,1-diyl, 1-propene-1,2-diyl, and 1-propene-1,3-diyl), butenediyl (including all isomeric forms, e.g., 1-butene-1,1-diyl, 1-butene-1,2-diyl, and 1-butene-1,4-diyl), pentenediyl (including all isomeric forms, e.g., 1-pentene-1,1-diyl, 1-pentene-1,2-diyl, and 1-pentene-1,5-diyl), and hexenediyl (including all isomeric forms, e.g., 1-hexene-1,1-diyl, 1-hexene-1,2-diyl, 1-hexene-1,3-diyl, 1-hexene-1,4-diyl, 1-hexene-1,5-diyl, and 1-hexene-1,6-diyl).

The term “alkynyl” refers to a linear or branched monovalent hydrocarbon radical, which contains one or more, in one embodiment, one, two, three, or four, in another embodiment, one, carbon-carbon triple bond(s). An alkynyl group does not contain a carbon-carbon double bond. The alkynyl is optionally substituted with one or more substituents Q as described herein. For example, C₂₋₆ alkynyl refers to a linear unsaturated monovalent hydrocarbon radical of 2 to 6 carbon atoms or a branched unsaturated monovalent hydrocarbon radical of 4 to 6 carbon atoms. In certain embodiments, the alkynyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 4 to 20 (C₄₋₂₀), 4 to 15 (C₄₋₁₅), 4 to 10 (C₄₋₁₀), or 4 to 6 (C₄₋₆) carbon atoms. Examples of alkynyl groups include, but are not limited to, ethynyl (—C≡CH), propynyl (including all isomeric forms, e.g., 1-propynyl (—C≡CCH₃) and propargyl (—CH₂C≡CH)), butynyl (including all isomeric forms, e.g., 1-butyn-1-yl and 2-butyn-1-yl), pentynyl (including all isomeric forms, e.g., 1-pentyn-1-yl and 1-methyl-2-butyn-1-yl), and hexynyl (including all isomeric forms, e.g., 1-hexyn-1-yl and 2-hexyn-1-yl).

The terms “alkynylene” and “alkynediyl” are used interchangeably herein in reference to a linear or branched divalent hydrocarbon radical, which contains one or more, in one embodiment, one, two, three, or four, in another embodiment, one, carbon-carbon triple bond(s). An alkynylene group does not contain a carbon-carbon double bond. The alkynediyl is optionally substituted with one or more substituents Q as described herein. For example, C₂₋₆ alkynediyl refers to a linear unsaturated divalent hydrocarbon radical of 2 to 6 carbon atoms or a branched unsaturated divalent hydrocarbon radical of 4 to 6 carbon atoms. In certain embodiments, the alkynediyl is a linear divalent hydrocarbon radical of 2 to 30 (C₂₋₃₀), 2 to 20 (C₂₋₂₀), 2 to 15 (C₂₋₁₅), 2 to 10 (C₂₋₁₀), or 2 to 6 (C₂₋₆) carbon atoms, or a branched divalent hydrocarbon radical of 4 to 30 (C₄₋₃₀), 4 to 20 (C₄₋₂₀), 4 to 15 (C₄₋₁₅), 4 to 10 (C₄₋₁₀), or 4 to 6 (C₄₋₆) carbon atoms. Examples of alkynediyl groups include, but are not limited to, ethynediyl, propynediyl (including all isomeric forms, e.g., 1-propyne-1,3-diyl and 1-propyne-3,3-diyl), butynediyl (including all isomeric forms, e.g., 1-butyne-1,3-diyl, 1-butyne-1,4-diyl, and 2-butyne-1,1-diyl), pentynediyl (including all isomeric forms, e.g., 1-pentyne-1,3-diyl, 1-pentyne-1,4-diyl, and 2-pentyne-1,1-diyl), and hexynediyl (including all isomeric forms, e.g., 1-hexyne-1,3-diyl, 1-hexyne-1,4-diyl, and 2-hexyne-1,1-diyl).

The term “cycloalkyl” refers to a cyclic monovalent hydrocarbon radical, which is optionally substituted with one or more substituents Q as described herein. In one embodiment, the cycloalkyl is a saturated or unsaturated but non-aromatic, and/or bridged or non-bridged, and/or fused bicyclic group. In certain embodiments, the cycloalkyl has from 3 to 20 (C₃₋₂₀), from 3 to 15 (C₃₋₁₅), from 3 to 10 (C₃₋₁₀), or from 3 to 7 (C₃₋₇) carbon atoms. In one embodiment, the cycloalkyl is monocyclic. In another embodiment, the cycloalkyl is bicyclic. In yet another embodiment, the cycloalkyl is tricyclic. In still another embodiment, the cycloalkyl is polycyclic. Examples of cycloalkyl groups include, but are not limited to, cyclopropyl, cyclobutyl, cyclopentyl, cyclopentenyl, cyclohexyl, cyclohexenyl, cyclohexadienyl, cycloheptyl, cycloheptenyl, bicyclo[1.1.1]pentyl, bicyclo[2.1.1]hexyl, bicyclo[2.2.1]heptyl, bicyclo[2.2.2]octyl, decalinyl, and adamantyl.

The term “aryl” refers to a monovalent monocyclic aromatic hydrocarbon radical and/or monovalent polycyclic aromatic hydrocarbon radical that contain at least one aromatic carbon ring. In certain embodiments, the aryl has from 6 to 20 (C₆₋₂₀), from 6 to 15 (C₆₋₁₅), or from 6 to 10 (C₆₋₁₀) ring carbon atoms. Examples of aryl groups include, but are not limited to, phenyl, naphthyl, fluorenyl, azulenyl, anthryl, phenanthryl, pyrenyl, biphenyl, and terphenyl. The aryl also refers to bicyclic or tricyclic carbon rings, where one of the rings is aromatic and the others of which may be saturated, partially unsaturated, or aromatic, for example, dihydronaphthyl, indenyl, indanyl, or tetrahydronaphthyl (tetralinyl). In one embodiment, the aryl is monocyclic. In another embodiment, the aryl is bicyclic. In yet another embodiment, the aryl is tricyclic. In still another embodiment, the aryl is polycyclic. In certain embodiments, the aryl is optionally substituted with one or more substituents Q as described herein.

The term “aralkyl” or “arylalkyl” refers to a monovalent alkyl group substituted with one or more aryl groups. In certain embodiments, the aralkyl has from 7 to 30 (C₇₋₃₀), from 7 to 20 (C₇₋₂₀), or from 7 to 16 (C₇₋₁₆) carbon atoms. Examples of aralkyl groups include, but are not limited to, benzyl, phenylethyl (including all isomeric forms, e.g., 1-phenylethyl and 2-phenylethyl), and phenylpropyl (including all isomeric forms, e.g., 1-phenylpropyl, 2-phenylpropyl, and 3-phenylpropyl). In certain embodiments, the aralkyl is optionally substituted with one or more substituents Q as described herein.

The term “heteroaryl” refers to a monovalent monocyclic aromatic group or monovalent polycyclic aromatic group that contain at least one aromatic ring, wherein at least one aromatic ring contains one or more heteroatoms, each independently selected from O, S, and N, in the ring. For a heteroaryl group containing a heteroaromatic ring and a nonaromatic heterocyclic ring, the heteroaryl group is not bonded to the rest of a molecule through its nonaromatic heterocyclic ring. Each ring of a heteroaryl group can contain one or two O atoms, one or two S atoms, and/or one to four N atoms; provided that the total number of heteroatoms in each ring is four or less and each ring contains at least one carbon atom. In certain embodiments, the heteroaryl has from 5 to 20, from 5 to 15, or from 5 to 10 ring atoms. In one embodiment, the heteroaryl is monocyclic. Examples of monocyclic heteroaryl groups include, but are not limited to, furanyl, imidazolyl, isothiazolyl, isoxazolyl, oxadiazolyl, oxazolyl, pyrazinyl, pyrazolyl, pyridazinyl, pyridyl, pyrimidinyl, pyrrolyl, thiadiazolyl, thiazolyl, thienyl, tetrazolyl, triazinyl, and triazolyl. In another embodiment, the heteroaryl is bicyclic. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzotriazolyl, benzoxazolyl, furopyrindyl (including all isomeric forms, e.g., furo[2,3-b]pyridinyl, furo[2,3-c]pyridinyl, furo[3,2-b]pyridinyl, furo[3,2-c]pyridinyl, furo[3,4-b]pyridinyl, and furo[3,4-c]pyridinyl), imidazopyridinyl (including all isomeric forms, e.g., imidazo[1,2-a]pyridinyl, imidazo[4,5-b]pyridinyl, and imidazo[4,5-c]pyridinyl), imidazothiazolyl (including all isomeric forms, e.g., imidazo[2,1-b]thiazolyl and imidazo[4,5-d]thiazolyl), indazolyl, indolizinyl, indolyl, isobenzofuranyl, isobenzothienyl (i.e., benzo[c]thienyl), isoindolyl, isoquinolinyl, naphthyridinyl (including all isomeric forms, e.g., 1,5-naphthyridinyl, 1,6-naphthyridinyl, 1,7-naphthyridinyl, and 1,8-naphthyridinyl), oxazolopyridinyl (including all isomeric forms, e.g., oxazolo[4,5-b]pyridinyl, oxazolo[4,5-c]pyridinyl, oxazolo[5,4-b]pyridinyl, and oxazolo[5,4-c]pyridinyl), phthalazinyl, pteridinyl, purinyl, pyrrolopyridyl (including all isomeric forms, e.g., pyrrolo[2,3-b]pyridinyl, pyrrolo[2,3-c]pyridinyl, pyrrolo[3,2-b]pyridinyl, and pyrrolo[3,2-c]pyridinyl), quinolinyl, quinoxalinyl, quinazolinyl, thiadiazolopyrimidyl (including all isomeric forms, e.g., [1,2,5]thiadiazolo[3,4-d]pyrimidinyl and [1,2,3]thiadiazolo[4,5-d]pyrimidinyl), and thienopyridyl (including all isomeric forms, e.g., thieno[2,3-b]pyridinyl, thieno[2,3-c]pyridinyl, thieno[3,2-b]pyridinyl, and thieno[3,2-c]pyridinyl). In yet another embodiment, the heteroaryl is tricyclic. Examples of tricyclic heteroaryl groups include, but are not limited to, acridinyl, benzindolyl, carbazolyl, dibenzofuranyl, perimidinyl, phenanthrolinyl, phenanthridinyl (including all isomeric forms, e.g., 1,5-phenanthrolinyl, 1,6-phenanthrolinyl, 1,7-phenanthrolinyl, 1,9-phenanthrolinyl, and 2,10-phenanthrolinyl), phenarsazinyl, phenazinyl, phenothiazinyl, phenoxazinyl, and xanthenyl. In certain embodiments, the heteroaryl is optionally substituted with one or more substituents Q as described herein.

The term “heterocyclyl” or “heterocyclic” refers to a monovalent monocyclic non-aromatic ring system or monovalent polycyclic ring system that contains at least one non-aromatic ring, wherein one or more of the non-aromatic ring atoms are heteroatoms, each independently selected from O, S, and N; and the remaining ring atoms are carbon atoms. For a heterocyclyl group containing a heteroaromatic ring and a nonaromatic heterocyclic ring, the heterocyclyl group is not bonded to the rest of a molecule through the heteroaromatic ring. In certain embodiments, the heterocyclyl or heterocyclic group has from 3 to 20, from 3 to 15, from 3 to 10, from 3 to 8, from 4 to 7, or from 5 to 6 ring atoms. In certain embodiments, the heterocyclyl is a monocyclic, bicyclic, tricyclic, or tetracyclic ring system, which may be fused or bridged, and in which nitrogen or sulfur atoms may be optionally oxidized, nitrogen atoms may be optionally quaternized, and some rings may be partially or fully saturated, or aromatic. The heterocyclyl may be attached to the main structure at any heteroatom or carbon atom which results in the creation of a stable compound. Examples of heterocyclyls and heterocyclic groups include, but are not limited to, azepinyl, benzodioxanyl, benzodioxolyl, benzofuranonyl, chromanyl, decahydroisoquinolinyl, dihydrobenzofuranyl, dihydrobenzisothiazolyl, dihydrobenzisoxazinyl (including all isomeric forms, e.g., 1,4-dihydrobenzo[d][1,3]oxazinyl, 3,4-dihydrobenzo[c][1,2]-oxazinyl, and 3,4-dihydrobenzo[d][1,2]oxazinyl), dihydrobenzothienyl, dihydroisobenzofuranyl, dihydrobenzo[c]thienyl, dihydrofuryl, dihydroisoindolyl, dihydropyranyl, dihydropyrazolyl, dihydropyrazinyl, dihydropyridinyl, dihydropyrimidinyl, dihydropyrrolyl, dioxolanyl, 1,4-dithianyl, furanonyl, imidazolidinyl, imidazolinyl, indolinyl, isochromanyl, isoindolinyl, isothiazolidinyl, isoxazolidinyl, morpholinyl, octahydroindolyl, octahydroisoindolyl, oxazolidinonyl, oxazolidinyl, oxiranyl, piperazinyl, piperidinyl, 4-piperidonyl, pyrazolidinyl, pyrazolinyl, pyrrolidinyl, pyrrolinyl, quinuclidinyl, tetrahydrofuryl, tetrahydroisoquinolinyl, tetrahydropyranyl, tetrahydrothienyl, thiamorpholinyl, thiazolidinyl, thiochromanyl, tetrahydroquinolinyl, and 1,3,5-trithianyl. In certain embodiments, the heterocyclyl is optionally substituted with one or more substituents Q as described herein.

The term “halogen,” “halide,” or “halo” refers to fluoro, chloro, bromo, and/or iodo.

The term “optionally substituted” is intended to mean that a group or substituent, such as an alkyl, alkylene, heteroalkyl, heteroalkylene, alkenyl, alkenylene, alkynyl, alkynylene, cycloalkyl, aryl, aralkyl, heteroaryl, or heterocyclyl group, may be substituted with one or more, in one embodiment, one, two, three, or four, substituents Q, each of which is independently selected from, e.g., (a) deuterium (-D), cyano (—CN), halo, imino (═NH), nitro (—NO₂), and oxo (═O); (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{b})OR^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl optionally substituted with one or more, in one embodiment, one, two, three, or four, substituents Q^{a}. As used herein, all groups that can be substituted are “optionally substituted.”

In one embodiment, each Q^{a} is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OP(O)(OR^{f})OR^{g}, —OS(O)R^{e}, —OS(O)₂R^{e}, —OS(O)NR^{f}R^{g}, —OS(O)₂NR^{f}R^{g}, —N^{f}R^{g}, —NC(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —N^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In certain embodiments, “optically active” and “enantiomerically active” refer to a collection of molecules, which has an enantiomeric excess of no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%. In certain embodiments, an optically active compound comprises about 95% or more of one enantiomer and about 5% or less of the other enantiomer based on the total weight of the enantiomeric mixture in question. In certain embodiments, an optically active compound comprises about 98% or more of one enantiomer and about 2% or less of the other enantiomer based on the total weight of the enantiomeric mixture in question. In certain embodiments, an optically active compound comprises about 99% or more of one enantiomer and about 1% or less of the other enantiomer based on the total weight of the enantiomeric mixture in question.

In describing an optically active compound, the prefixes R and S are used to denote the absolute configuration of the compound about its chiral center(s). The (+) and (−) are used to denote the optical rotation of the compound, that is, the direction in which a plane of polarized light is rotated by the optically active compound. The (−) prefix indicates that the compound is levorotatory, that is, the compound rotates the plane of polarized light to the left or counterclockwise. The (+) prefix indicates that the compound is dextrorotatory, that is, the compound rotates the plane of polarized light to the right or clockwise. However, the sign of optical rotation, (+) and (−), is not related to the absolute configuration of the compound, R and S.

The term “isotopically enriched” refers to a compound that contains an unnatural proportion of an isotope at one or more of the atoms that constitute such a compound. In certain embodiments, an isotopically enriched compound contains unnatural proportions of one or more isotopes, including, but not limited to, hydrogen (H), deuterium (²H), tritium (³H), carbon-11 (¹¹C), carbon-12 (²C), carbon-13 (³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I), iodine-125 (¹²⁵I), iodine-127 (¹²⁷I), iodine-129 (¹²⁹I), and iodine-131 (¹³¹I). In certain embodiments, an isotopically enriched compound is in a stable form, that is, non-radioactive. In certain embodiments, an isotopically enriched compound contains unnatural proportions of one or more isotopes, including, but not limited to, hydrogen (¹H), deuterium (2H), carbon-12 (¹²C), carbon-13 (¹³C), nitrogen-14 (¹⁴N), nitrogen-15 (¹⁵N), oxygen-16 (¹⁶O), oxygen-17 (¹⁷O), oxygen-18 (¹⁸O), fluorine-17 (¹⁷F), phosphorus-31 (³¹P), sulfur-32 (³²S), sulfur-33 (³³S), sulfur-34 (³⁴S), sulfur-36 (³⁶S), chlorine-35 (³⁵Cl), chlorine-37 (³⁷Cl), bromine-79 (⁷⁹Br), bromine-81 (⁸¹Br), and iodine-127 (¹²⁷I). In certain embodiments, an isotopically enriched compound is in an unstable form, that is, radioactive. In certain embodiments, an isotopically enriched compound contains unnatural proportions of one or more isotopes, including, but not limited to, tritium (³H), carbon-11 (¹¹C), carbon-14 (¹⁴C), nitrogen-13 (¹³N), oxygen-14 (¹⁴O), oxygen-15 (¹⁵O), fluorine-18 (¹⁸F), phosphorus-32 (³²P), phosphorus-33 (³³P) sulfur-35 (³⁵S), chlorine-36 (³⁶Cl), iodine-123 (¹²³I), iodine-125 (¹²¹I), iodine-129 (¹²⁹I), and iodine-131 (¹³¹I). It will be understood that, in a compound as provided herein, any hydrogen can be ²H, as example, or any carbon can be ¹³C, as example, or any nitrogen can be ¹⁵N, as example, or any oxygen can be ¹⁸O, as example, where feasible according to the judgment of one of ordinary skill in the art.

The term “isotopic enrichment” refers to the percentage of incorporation of a less prevalent isotope (e.g., D for deuterium or hydrogen-2) of an element at a given position in a molecule in the place of a more prevalent isotope (e.g., ¹H for protium or hydrogen-1) of the element. As used herein, when an atom at a particular position in a molecule is designated as a particular less prevalent isotope, it is understood that the abundance of that isotope at that position is substantially greater than its natural abundance.

The term “isotopic enrichment factor” refers the ratio between the isotopic abundance in an isotopically enriched compound and the natural abundance of a specific isotope.

The term “hydrogen” or the symbol “H” refers to the composition of naturally occurring hydrogen isotopes, which include protium (¹H), deuterium (²H or D), and tritium (³H), in their natural abundances. Protium is the most common hydrogen isotope having a natural abundance of more than 99.98%. Deuterium is a less prevalent hydrogen isotope having a natural abundance of about 0.0156%.

The term “deuterium enrichment” refers to the percentage of incorporation of deuterium at a given position in a molecule in the place of hydrogen. For example, deuterium enrichment of 1% at a given position means that 1% of molecules in a given sample contain deuterium at the specified position. Because the naturally occurring distribution of deuterium is about 0.0156% on average, deuterium enrichment at any position in a compound synthesized using non-enriched starting materials is about 0.0156% on average. As used herein, when a particular position in an isotopically enriched compound is designated as having deuterium, it is understood that the abundance of deuterium at that position in the compound is substantially greater than its natural abundance (0.0156%).

The term “carbon” or the symbol “C” refers to the composition of naturally occurring carbon isotopes, which include carbon-12 (¹²C) and carbon-13 (¹³C) in their natural abundances. Carbon-12 is the most common carbon isotope having a natural abundance of more than 98.89%. Carbon-13 is a less prevalent carbon isotope having a natural abundance of about 1.11%.

The term “carbon-13 enrichment” or “¹³C enrichment” refers to the percentage of incorporation of carbon-13 at a given position in a molecule in the place of carbon. For example, carbon-13 enrichment of 10% at a given position means that 10% of molecules in a given sample contain carbon-13 at the specified position. Because the naturally occurring distribution of carbon-13 is about 1.11% on average, carbon-13 enrichment at any position in a compound synthesized using non-enriched starting materials is about 1.11% on average. As used herein, when a particular position in an isotopically enriched compound is designated as having carbon-13, it is understood that the abundance of carbon-13 at that position in the compound is substantially greater than its natural abundance (1.11%).

The terms “substantially pure” and “substantially homogeneous” mean, when referred to a substance, sufficiently homogeneous to appear free of readily detectable impurities as determined by a standard analytical method used by one of ordinary skill in the art, including, but not limited to, thin layer chromatography (TLC), gel electrophoresis, high performance liquid chromatography (HPLC), gas chromatography (GC), nuclear magnetic resonance (NMR), and mass spectrometry (MS); or sufficiently pure such that further purification would not detectably alter the physical, chemical, biological, and/or pharmacological properties, such as enzymatic and biological activities, of the substance. In certain embodiments, “substantially pure” or “substantially homogeneous” refers to a collection of molecules, wherein at least about 95%, at least about 96%, at least about 97%, at least about 98%, at least about 99%, or at least about 99.5% by weight of the molecules are a single compound, including a single enantiomer, a racemic mixture, or a mixture of enantiomers, as determined by standard analytical methods. As used herein, when an atom at a particular position in an isotopically enriched molecule is designated as a particular less prevalent isotope, a molecule that contains other than the designated isotope at the specified position is an impurity with respect to the isotopically enriched compound. Thus, for a deuterated compound that has an atom at a particular position designated as deuterium, a compound that contains a protium at the same position is an impurity.

The term “solvate” refers to a complex or aggregate formed by one or more molecules of a solute, e.g., a compound provided herein, and one or more molecules of a solvent, which are present in a stoichiometric or non-stoichiometric amount. Suitable solvents include, but are not limited to, water, methanol, ethanol, n-propanol, isopropanol, and acetic acid. In certain embodiments, the solvent is pharmaceutically acceptable. In one embodiment, the complex or aggregate is in a crystalline form. In another embodiment, the complex or aggregate is in a noncrystalline form. Where the solvent is water, the solvate is a hydrate. Examples of hydrates include, but are not limited to, a hemihydrate, monohydrate, dihydrate, trihydrate, tetrahydrate, and pentahydrate.

For a divalent group described herein, no orientation is implied by the direction in which the divalent group is presented. For example, unless a particular orientation is specified, the formula —C(O)O— represents both —C(O)O— and —OC(O)—.

The phrase “an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof” has the same meaning as the phrase “(i) an enantiomer, a mixture of enantiomers, a diastereomer, a mixture of two or more diastereomers, a tautomer, a mixture of two or more tautomers, or an isotopic variant of the compound referenced therein; (ii) a pharmaceutically acceptable salt, solvate, or hydrate of the compound referenced therein; or (iii) a pharmaceutically acceptable salt, solvate, or hydrate of an enantiomer, a mixture of enantiomers, a diastereomer, a mixture of two or more diastereomers, a tautomer, a mixture of two or more tautomers, or an isotopic variant of the compound referenced therein.”

### Compounds

In one embodiment, provided herein is a compound of Formula (I):

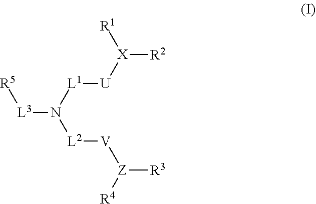

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof; wherein:
R¹ is hydrogen, C₁₋₂₀ alkyl, C₁₋₂₀ heteroalkyl, C₂₋₂₀ alkenyl, or C₂₋₂₀ alkynyl;
R², R³, and R⁴ are each independently C₁₋₂₀ alkyl, C₁₋₂₀ heteroalkyl, C₂₋₂₀ alkenyl, or C₂₋₂₀ alkynyl;
R⁵ is —C(O)OR^{5a}, —OR^{5a}, or —OC(O)R^{5a}; wherein each R^{5a} is independently hydrogen, C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl;
L¹, L², and L³ are each independently C₁₋₂₀ alkylene, C₁₋₂₀ heteroalkylene, C₂₋₂₀ alkenylene, or C₂₋₂₀ alkynylene;
U and V are each independently —C(O)—, —C(O)O—, or —OC(O)O—; and
X and Y are each independently C(H) or N;
with the proviso that, when U and V are each —C(O)O—, and X and Z are each C(H), then at least one of R¹, R², R³, and R⁴ is substituted C₁₋₂₀ alkyl, or at least one of L¹ and L² is C₁₋₂₀ heteroalkylene;
wherein each alkyl, alkylene, heteroalkyl, heteroalkylene, alkenyl, alkenylene, alkynyl, alkynylene, cycloalkyl, aryl, aralkyl, heteroaryl, and heterocyclyl is optionally substituted with one or more, in one embodiment, one, two, three, or four, substituents Q, wherein each Q is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{b})OR^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, nitro, imino, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OP(O)(OR^{f})OR^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{f}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In certain embodiments, in Formula (I), U and V are each —C(O)O—; X and Z are each C(H); and R¹, R², R³, or R⁴ is C₁₋₂₀ alkyl, substituted with one or more substituents Q. In certain embodiments, in Formula (I), U and V are each —C(O)O—; X and Z are each C(H); and R¹, R², R³, or R⁴ is C₁₋₂₀ alkyl, substituted with one or more halo. In certain embodiments, in Formula (I), U and V are each —C(O)O—; X and Z are each C(H); and R¹, R², R³, or R⁴ is C₁₋₂₀ alkyl, substituted with one or more fluoro. In certain embodiments, in Formula (I), U and V are each —C(O)O—; X and Z are each C(H); and R¹, R², R³, or R⁴ is C₁₋₂₀ alkyl, substituted with one, two, or three fluoro.

In certain embodiments, in Formula (I), U and V are each —C(O)O—; X and Z are each C(H); and L¹ or L² is C₁₋₂₀ heteroalkylene, optionally substituted with one or more substituents Q. In certain embodiments, in Formula (I), U and V are each —C(O)O—; X and Z are each C(H); and L¹ or L² is unsubstituted C₁₋₂₀ heteroalkylene. In certain embodiments, in Formula (I), U and V are each —C(O)O—; X and Z are each C(H); and L¹ and L² are each independently —NH—C₁₋₂₀ alkylene or —NH—C₁₋₂₀ heteroalkylene, each optionally substituted with one or more substituents Q.

In certain embodiments, in Formula (I), U and V are each —C(O)O—; X and Z are each C(H); R¹, R², R³, or R⁴ is C₁₋₂₀ alkyl, substituted with one or more substituents Q; and L¹ or L² is C₁₋₂₀ heteroalkylene, optionally substituted with one or more substituents Q. In certain embodiments, in Formula (I), U and V are each —C(O)O—; X and Z are each C(H); R¹, R², R³, or R⁴ is C₁₋₂₀ alkyl, substituted with one or more halo; and L¹ or L² is unsubstituted C₁₋₂₀ heteroalkylene. In certain embodiments, in Formula (I), U and V are each —C(O)O—; X and Z are each C(H); R¹, R², R³, or R⁴ is C₁₋₂₀ alkyl, substituted with one or more fluoro; and L¹ or L² is unsubstituted C₁₋₂₀ heteroalkylene. In certain embodiments, in Formula (I), U and V are each —C(O)O—; X and Z are each C(H); R¹, R², R³, or R⁴ is C₁₋₂₀ alkyl, substituted with one, two, or three fluoro; and L¹ or L² is unsubstituted C₁₋₂₀ heteroalkylene.

In certain embodiments, in Formula (I), U and V are each —C(O)—; X and Z are each C(H); and L¹ and L² are each independently C₁₋₂₀ heteroalkylene, optionally substituted with one or more substituents Q. In certain embodiments, in Formula (I), U and V are each —C(O)—; X and Z are each C(H); and L¹ and L² are each independently unsubstituted C₁₋₂₀ heteroalkylene. In certain embodiments, in Formula (I), U and V are each —C(O)—; X and Z are each C(H); and L¹ and L² are each independently —NH—C₁₋₂₀ alkylene or —NH—C₁₋₂₀ hetero-alkylene, each optionally substituted with one or more substituents Q.

In one embodiment, in Formula (I),
R¹ is hydrogen or C₁₋₁₀ alkyl, optionally substituted with one or more substituents Q;
R², R³, and R⁴ are each independently C₁₋₁₀ alkyl, optionally substituted with one or more substituents Q;
R¹ is hydroxyl;
L¹ and L² are each independently C₁₋₁₀ alkylene or C₁₋₁₀ heteroalkylene, each optionally substituted with one or more substituents Q;
L¹ is C₁₋₁₀ alkylene, optionally substituted with one or more substituents Q;
U and V are each independently —C(O)—, —C(O)O—, or —OC(O)O—; and
X and Y are each independently C(H) or N.

In another embodiment, in Formula (I),
R¹ is hydrogen, unsubstituted C₁₋₁₀ alkyl, or C₁₋₁₀ alkyl substituted with one, two, or three substituents halo;
R², R³, and R⁴ are each independently unsubstituted C₁₋₁₀ alkyl, or C₁₋₁₀ alkyl substituted with one, two, or three substituents halo;
R⁵ is hydroxyl;
L¹ and L² are each independently unsubstituted C₁₋₁₀ alkylene or unsubstituted C₁₋₁₀ heteroalkylene;
L³ is unsubstituted C₁₋₁₀ alkylene;
U and V are each independently —C(O)—, —C(O)O—, or —OC(O)O—; and
X and Y are each independently C(H) or N.

In yet another embodiment, in Formula (I),
R¹ is hydrogen, hexyl, octyl, decyl, trifluorohexyl, trifluorooctyl, or trifluorodecyl;
R², R³, and R⁴ are each independently hexyl, octyl, decyl, trifluorohexyl, trifluorooctyl, or trifluorodecyl;
R⁵ is hydroxyl;
L¹ and L² are each independently pentanediyl, hexanediyl, heptanediyl, —(CH₂)₂O(CH₂)₂—, —(CH₂)₂O(CH₂)₃—, or —(CH₂)₂O(CH₂)₂O(CH₂)₂—;
L³ is ethanediyl, propanediyl, butanediyl, or pentanediyl;
U and V are each independently —C(O)—, —C(O)O—, or —OC(O)O—; and
X and Y are each independently C(H) or N.

In yet another embodiment, in Formula (I),
R¹ is hydrogen, hexyl, octyl, decyl, trifluorohexyl, trifluorooctyl, or trifluorodecyl;
R², R³, and R⁴ are each independently hexyl, octyl, decyl, trifluorohexyl, trifluorooctyl, or trifluorodecyl;
R⁵ is hydroxyl;
L¹ and L² are each independently pentanediyl, hexanediyl, heptanediyl, or —(CH₂)₂O(CH₂)₃—;
L³ is ethanediyl, propanediyl, butanediyl, or pentanediyl;
U and V are each independently —C(O)—, —C(O)O—, or —OC(O)O—; and
X and Y are each independently C(H) or N.

In yet another embodiment, in Formula (I),
R¹ is hydrogen, n-hexyl, n-octyl, n-decyl, 6,6,6-trifluorohex-1-yl, 8,8,8-trifluorooct-1-yl, or 10,10,10-trifluorodec-1-yl;
R², R³, and R⁴ are each independently n-hexyl, n-octyl, n-decyl, 6,6,6-trifluorohex-1-yl, 8,8,8-trifluorooct-1-yl, or 10,10,10-trifluorodec-1-yl;
R⁵ is hydroxyl;
L¹ and L² are each independently pentane-1,5-diyl, hexane-1,6-diyl, heptane-1,7-diyl, —(CH₂)₂O(CH₂)₂—, —(CH₂)₂O(CH₂)₃—, or —(CH₂)₂O(CH₂)₂O(CH₂)₂—;
L³ is ethane-1,2-diyl, propane-1,3-diyl, butane-1,4-diyl, or pentane-1,5-diyl;
U and V are each independently —C(O)—, —C(O)O—, or —OC(O)O—; and
X and Y are each independently C(H) or N.

In yet another embodiment, in Formula (1),
R¹ is hydrogen, n-hexyl, n-octyl, n-decyl, 6,6,6-trifluorohex-1-yl, 8,8,8-trifluorooct-1-yl, or 10,10,10-trifluorodec-1-yl;
R², R³, and R⁴ are each independently n-hexyl, n-octyl, n-decyl, 6,6,6-trifluorohex-1-yl, 8,8,8-trifluorooct-1-yl, or 10,10,10-trifluorodec-1-yl;
R⁵ is hydroxyl;
L¹ and L² are each independently pentane-1,5-diyl, hexane-1,6-diyl, heptane-1,7-diyl, or —(CH₂)₂O(CH₂)₃—;
L³ is ethane-1,2-diyl, propane-1,3-diyl, butane-1,4-diyl, or pentane-1,5-diyl;
U and V are each independently —C(O)—, —C(O)O—, or —OC(O)O—; and
X and Y are each independently C(H) or N.

In yet another embodiment, in Formula (I),
R¹ is hydrogen, n-hexyl, n-octyl, 6,6,6-trifluorohex-1-yl, or 8,8,8-trifluorooct-1-yl;
R² is n-octyl, n-decyl, 8,8,8-trifluorooct-1-yl, or 10,10,10-trifluorodec-1-yl;
R³ is n-hexyl, n-octyl, 6,6,6-trifluorohex-1-yl, or 8,8,8-trifluorooct-1-yl;
R⁴ is n-octyl or 8,8,8-trifluorooct-1-yl;
R⁵ is hydroxyl;
L¹ and L² are each independently pentane-1,5-diyl, hexane-1,6-diyl, heptane-1,7-diyl, —(CH₂)₂O(CH₂)₂—, —(CH₂)₂O(CH₂)₃—, or —(CH₂)₂O(CH₂)₂O(CH₂)₂—;
L³ is ethane-1,2-diyl, propane-1,3-diyl, butane-1,4-diyl, or pentane-1,5-diyl;
U and V are each independently —C(O)—, —C(O)O—, or —OC(O)O—; and
X and Y are each independently C(H) or N.

In still another embodiment, in Formula (I),
R¹ is hydrogen, n-hexyl, n-octyl, 6,6,6-trifluorohex-1-yl, or 8,8,8-trifluorooct-1-yl;
R² is n-octyl, n-decyl, 8,8,8-trifluorooct-1-yl, or 10,10,10-trifluorodec-1-yl;
R³ is n-hexyl, n-octyl, 6,6,6-trifluorohex-1-yl, or 8,8,8-trifluorooct-1-yl;
R⁴ is n-octyl or 8,8,8-trifluorooct-1-yl;
R⁵ is hydroxyl;
L¹ and L² are each independently pentane-1,5-diyl, hexane-1,6-diyl, heptane-1,7-diyl, or —(CH₂)₂O(CH₂)₃—;
L³ is ethane-1,2-diyl, propane-1,3-diyl, butane-1,4-diyl, or pentane-1,5-diyl;
U and V are each independently —C(O)—, —C(O)O—, or —OC(O)O—; and
X and Y are each independently C(H) or N.

In one embodiment, in Formula (I),
R¹ is hydrogen or C₁₋₁₀ alkyl, optionally substituted with one or more substituents Q;
R², R³, and R⁴ are each independently C₁₋₂₀ alkyl or C₁₋₂₀ heteroalkyl, optionally substituted with one or more substituents Q;
R⁵ is hydroxyl;
L¹ and L² are each independently C₁₋₁₀ alkylene or C₁₋₁₀ heteroalkylene, each optionally substituted with one or more substituents Q;
L³ is C₁₋₁₀ alkylene, optionally substituted with one or more substituents Q;
U and V are each independently —C(O)—, —C(O)O—, or —OC(O)O—; and
X and Y are each independently C(H) or N.

In another embodiment, in Formula (I),
R¹ is hydrogen, hexyl, octyl, decyl, trifluorohexyl, trifluorooctyl, or trifluorodecyl;
R², R³, and R⁴ are each independently hexyl, octyl, decyl, trifluorohexyl, trifluorooctyl, trifluorodecyl, —(CH₂)₂O(CH₂)₂O(CH₂)₄CH₃, or —(CH₂)₂O(CH₂)₂O(CH₂)₂O—(CH₂)₁₀CH₃;
R⁵ is hydroxyl;
L¹ and L² are each independently pentanediyl, hexanediyl, heptanediyl, —(CH₂)₂O(CH₂)₂—, —(CH₂)₂O(CH₂)₃—, —(CH₂)₂O(CH₂)₂O(CH₂)₂—, or —(CH₂)₆N(H)—;
L³ is ethanediyl, propanediyl, butanediyl, or pentanediyl;
U and V are each independently —C(O)—, —C(O)O—, or —OC(O)O—; and
X and Y are each independently C(H) or N.

In yet another embodiment, in Formula (I),
R¹ is hydrogen, n-hexyl, n-octyl, n-decyl, 6,6,6-trifluorohex-1-yl, 8,8,8-trifluorooct-1-yl, or 10,10,10-trifluorodec-1-yl;
R², R³, and R⁴ are each independently n-hexyl, n-octyl, n-decyl, 6,6,6-trifluorohex-1-yl, 8,8,8-trifluorooct-1-yl, 10,10,10-trifluorodec-1-yl, —(CH₂)₂O(CH₂)₂O(CH₂)₄—CH₃, or —(CH₂)₂O(CH₂)₂O(CH₂)₂O(CH₂)₁₀CH₃;
R⁵ is hydroxyl;
L¹ and L² are each independently pentane-1,5-diyl, hexane-1,6-diyl, heptane-1,7-diyl, —(CH₂)₂O(CH₂)₂—, —(CH₂)₂O(CH₂)₃—, —(CH₂)₂O(CH₂)₂O(CH₂)₂—, or —(CH₂)₆N(H)—;
L³ is ethane-1,2-diyl, propane-1,3-diyl, butane-1,4-diyl, or pentane-1,5-diyl;
U and V are each independently —C(O)—, —C(O)O—, or —OC(O)O—; and
X and Y are each independently C(H) or N.

In still another embodiment, in Formula (I),
R¹ is hydrogen, n-hexyl, n-octyl, 6,6,6-trifluorohex-1-yl, or 8,8,8-trifluorooct-1-yl;
R² is n-octyl, n-decyl, 8,8,8-trifluorooct-1-yl, 10,10,10-trifluorodec-1-yl, —(CH₂)₂O(CH₂)₂O(CH₂)₄CH₃, or —(CH₂)₂O(CH₂)₂O(CH₂)₂O(CH₂)₁₀CH₃;
R³ is n-hexyl, n-octyl, 6,6,6-trifluorohex-1-yl, or 8,8,8-trifluorooct-1-yl;
R⁴ is n-octyl or 8,8,8-trifluorooct-1-yl;
R⁵ is hydroxyl;
L¹ and L² are each independently pentane-1,5-diyl, hexane-1,6-diyl, heptane-1,7-diyl, —(CH₂)₂O(CH₂)₂—, —(CH₂)₂O(CH₂)₃—, —(CH₂)₂O(CH₂)₂O(CH₂)₂—, or —(CH₂)₆N(H)—;
L³ is ethane-1,2-diyl, propane-1,3-diyl, butane-1,4-diyl, or pentane-1,5-diyl;
U and V are each independently —C(O)—, —C(O)O—, or —OC(O)O—; and
X and Y are each independently C(H) or N.

In another embodiment, provided herein is a compound of Formula (II):

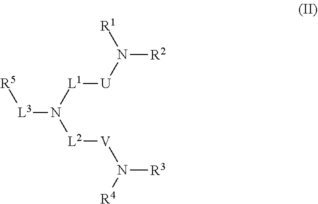

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, or hydrate thereof; wherein U and V are each independently —C(O)— or —C(O)O—; and R¹, R², R³, R⁴, R⁵, L¹, L², and L³ are each as defined herein.

In one embodiment, in Formula (II),
R¹ is hydrogen or C₁₋₁₀ alkyl, optionally substituted with one or more substituents Q;
R², R³, and R⁴ are each independently C₁₋₁₀ alkyl, optionally substituted with one or more substituents Q;
R⁵ is hydroxyl;
L¹ and L² are each independently C₁₋₁₀ alkylene or C₁₋₁₀ heteroalkylene, each optionally substituted with one or more substituents Q;
L³ is C₁₋₁₀ alkylene, optionally substituted with one or more substituents Q; and
U and V are each independently —C(O)— or —C(O)O—.

In another embodiment, in Formula (11),
R¹ is hydrogen, unsubstituted C₁₋₁₀ alkyl, or C₁₋₁₀ alkyl substituted with one, two, or three substituents halo;
R², R³, and R⁴ are each independently unsubstituted C₁₋₁₀ alkyl, or C₁₋₁₀ alkyl substituted with one, two, or three substituents halo;
R⁵ is hydroxyl;
L¹ and L² are each independently unsubstituted C₁₋₁₀ alkylene or unsubstituted C₁₋₁₀ heteroalkylene;
L³ is unsubstituted C₁₋₁₀ alkylene; and
U and V are each independently —C(O)— or —C(O)O—.

In yet another embodiment, in Formula (II),
R¹ is hydrogen, hexyl, octyl, decyl, trifluorohexyl, trifluorooctyl, or trifluorodecyl;
R², R³, and R⁴ are each independently hexyl, octyl, decyl, trifluorohexyl, trifluorooctyl, or trifluorodecyl;
R⁵ is hydroxyl;
L¹ and L² are each independently pentanediyl, hexanediyl, heptanediyl, —(CH₂)₂O(CH₂)₂—, —(CH₂)₂O(CH₂)₃—, or —(CH₂)₂O(CH₂)₂O(CH₂)₂—;
L³ is ethanediyl, propanediyl, butanediyl, or pentanediyl; and
U and V are each independently —C(O)— or —C(O)O—.

In yet another embodiment, in Formula (II),
R¹ is hydrogen, hexyl, octyl, decyl, trifluorohexyl, trifluorooctyl, or trifluorodecyl;
R², R³, and R⁴ are each independently hexyl, octyl, decyl, trifluorohexyl, trifluorooctyl, or trifluorodecyl;
R⁵ is hydroxyl;
L¹ and L² are each independently pentanediyl, hexanediyl, heptanediyl, or —(CH₂)₂O(CH₂)₃—;
L³ is ethanediyl, propanediyl, butanediyl, or pentanediyl; and
U and V are each independently —C(O)— or —C(O)O—.

In yet another embodiment, in Formula (11),
R¹ is hydrogen, n-hexyl, n-octyl, n-decyl, 6,6,6-trifluorohex-1-yl, 8,8,8-trifluorooct-1-yl, or 10,10,10-trifluorodec-1-yl;
R², R³, and R⁴ are each independently n-hexyl, n-octyl, n-decyl, 6,6,6-trifluorohex-1-yl, 8,8,8-trifluorooct-1-yl, or 10,10,10-trifluorodec-1-yl;
R⁵ is hydroxyl;
L¹ and L² are each independently pentanediyl, hexanediyl, heptanediyl, —(CH₂)₂O(CH₂)₂—, —(CH₂)₂O(CH₂)₃—, or —(CH₂)₂O(CH₂)₂O(CH₂)₂—;
L³ is ethane-1,2-diyl, propane-1,3-diyl, butane-1,4-diyl, or pentane-1,5-diyl; and
U and V are each independently —C(O)— or —C(O)O—.

In yet another embodiment, in Formula (II),
R¹ is hydrogen, n-hexyl, n-octyl, n-decyl, 6,6,6-trifluorohex-1-yl, 8,8,8-trifluorooct-1-yl, or 10,10,10-trifluorodec-1-yl;
R², R³, and R⁴ are each independently n-hexyl, n-octyl, n-decyl, 6,6,6-trifluorohex-1-yl, 8,8,8-trifluorooct-1-yl, or 10,10,10-trifluorodec-1-yl;
R⁵ is hydroxyl;
L¹ and L² are each independently pentane-1,5-diyl, hexane-1,6-diyl, heptane-1,7-diyl, or —(CH₂)₂O(CH₂)₃—;
L³ is ethane-1,2-diyl, propane-1,3-diyl, butane-1,4-diyl, or pentane-1,5-diyl; and
U and V are each independently —C(O)— or —C(O)O—.

In yet another embodiment, in Formula (II),
R¹ is hydrogen, n-hexyl, n-octyl, 6,6,6-trifluorohex-1-yl, or 8,8,8-trifluorooct-1-yl;
R² is n-octyl, n-decyl, 8,8,8-trifluorooct-1-yl, or 10,10,10-trifluorodec-1-yl;
R³ is n-hexyl, n-octyl, 6,6,6-trifluorohex-1-yl, or 8,8,8-trifluorooct-1-yl;
R⁴ is n-octyl or 8,8,8-trifluorooct-1-yl;
R⁵ is hydroxyl;
L¹ and L² are each independently pentanediyl, hexanediyl, heptanediyl, —(CH₂)₂O(CH₂)₂—, —(CH₂)₂O(CH₂)₃—, or —(CH₂)₂O(CH₂)₂O(CH₂)₂—;
L³ is ethane-1,2-diyl, propane-1,3-diyl, butane-1,4-diyl, or pentane-1,5-diyl; and
U and V are each independently —C(O)— or —C(O)O—.

In still another embodiment, in Formula (II),
R¹ is hydrogen, n-hexyl, n-octyl, 6,6,6-trifluorohex-1-yl, or 8,8,8-trifluorooct-1-yl;
R² is n-octyl, n-decyl, 8,8,8-trifluorooct-1-yl, or 10,10,10-trifluorodec-1-yl;
R³ is n-hexyl, n-octyl, 6,6,6-trifluorohex-1-yl, or 8,8,8-trifluorooct-1-yl;
R⁴ is n-octyl or 8,8,8-trifluorooct-1-yl;
R⁵ is hydroxyl;
L¹ and L² are each independently pentane-1,5-diyl, hexane-1,6-diyl, heptane-1,7-diyl, or —(CH₂)₂O(CH₂)₃—;
L³ is ethane-1,2-diyl, propane-1,3-diyl, butane-1,4-diyl, or pentane-1,5-diyl; and
U and V are each independently —C(O)— or —C(O)O—.

In yet another embodiment, provided herein is a compound of Formula (III):

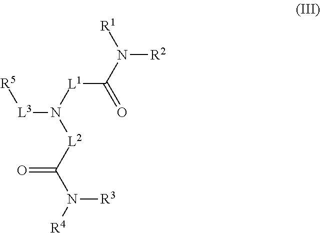

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R³, R⁴, R⁵, L¹, L², and L³ are each as defined herein.

In yet another embodiment, provided herein is a compound of Formula (IV):

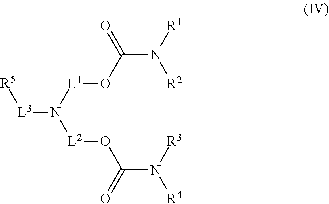

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, or hydrate thereof; wherein R¹, R², R³, R⁴, R⁵, L¹, L², and L³ are each as defined herein.

In one embodiment, in Formula (III) or (IV),
R¹ is hydrogen or C₁₋₁₀ alkyl, optionally substituted with one or more substituents Q;
R², R³, and R⁴ are each independently C₁₋₁₀ alkyl, optionally substituted with one or more substituents Q;
R⁵ is hydroxyl;
L¹ and L² are each independently C₁₋₁₀ alkylene or C₁₋₁₀ heteroalkylene, each optionally substituted with one or more substituents Q; and
L³ is C₁₋₁₀ alkylene, optionally substituted with one or more substituents Q.

In another embodiment, in Formula (III) or (IV),
R¹ is hydrogen, unsubstituted C₁₋₁₀ alkyl, or C₁₋₁₀ alkyl substituted with one, two, or three substituents halo;
R², R³, and R⁴ are each independently unsubstituted C₁₋₁₀ alkyl, or C₁₋₁₀ alkyl substituted with one, two, or three substituents halo;
R⁵ is hydroxyl;
L¹ and L² are each independently unsubstituted C₁₋₁₀ alkylene or unsubstituted C₁₋₁₀ heteroalkylene; and
L³ is unsubstituted C₁₋₁₀ alkylene.

In yet another embodiment, in Formula (III) or (IV),
R¹ is hydrogen, hexyl, octyl, decyl, trifluorohexyl, trifluorooctyl, or trifluorodecyl;
R², R³, and R⁴ are each independently hexyl, octyl, decyl, trifluorohexyl, trifluorooctyl, or trifluorodecyl;
R⁵ is hydroxyl;
L¹ and L² are each independently pentanediyl, hexanediyl, heptanediyl, —(CH₂)₂O(CH₂)₂—, —(CH₂)₂O(CH₂)₃—, or —(CH₂)₂O(CH₂)₂O(CH₂)₂—; and
L³ is ethanediyl, propanediyl, butanediyl, or pentanediyl.

In yet another embodiment, in Formula (111) or (IV),
R¹ is hydrogen, hexyl, octyl, decyl, trifluorohexyl, trifluorooctyl, or trifluorodecyl;
R², R³, and R⁴ are each independently hexyl, octyl, decyl, trifluorohexyl, trifluorooctyl, or trifluorodecyl;
R⁵ is hydroxyl;
L¹ and L² are each independently pentanediyl, hexanediyl, heptanediyl, or —(CH₂)₂O(CH₂)₃—; and
L³ is ethanediyl, propanediyl, butanediyl, or pentanediyl.

In yet another embodiment, in Formula (III) or (IV),
R¹ is hydrogen, n-hexyl, n-octyl, n-decyl, 6,6,6-trifluorohex-1-yl, 8,8,8-trifluorooct-1-yl, or 10,10,10-trifluorodec-1-yl;
R², R³, and R⁴ are each independently n-hexyl, n-octyl, n-decyl, 6,6,6-trifluorohex-1-yl, 8,8,8-trifluorooct-1-yl, or 10,10,10-trifluorodec-1-yl;
R⁵ is hydroxyl;
L¹ and L² are each independently pentanediyl, hexanediyl, heptanediyl, —(CH₂)₂O(CH₂)₂—, —(CH₂)₂O(CH₂)₃—, or —(CH₂)₂O(CH₂)₂O(CH₂)₂—;
and
L³ is ethane-1,2-diyl, propane-1,3-diyl, butane-1,4-diyl, or pentane-1,5-diyl.

In yet another embodiment, in Formula (III) or (IV),
R¹ is hydrogen, n-hexyl, n-octyl, n-decyl, 6,6,6-trifluorohex-1-yl, 8,8,8-trifluorooct-1-yl, or 10,10,10-trifluorodec-1-yl;
R², R³, and R⁴ are each independently n-hexyl, n-octyl, n-decyl, 6,6,6-trifluorohex-1-yl, 8,8,8-trifluorooct-1-yl, or 10,10,10-trifluorodec-1-yl;
R⁵ is hydroxyl;
L¹ and L² are each independently pentane-1,5-diyl, hexane-1,6-diyl, heptane-1,7-diyl, or —(CH₂)₂O(CH₂)₃—; and
L³ is ethane-1,2-diyl, propane-1,3-diyl, butane-1,4-diyl, or pentane-1,5-diyl.

In yet another embodiment, in Formula (III) or (IV),
R¹ is hydrogen, n-hexyl, n-octyl, 6,6,6-trifluorohex-1-yl, or 8,8,8-trifluorooct-1-yl;
R² is n-octyl, n-decyl, 8,8,8-trifluorooct-1-yl, or 10,10,10-trifluorodec-1-yl;
R³ is n-hexyl, n-octyl, 6,6,6-trifluorohex-1-yl, or 8,8,8-trifluorooct-1-yl;
R⁴ is n-octyl or 8,8,8-trifluorooct-1-yl;
R⁵ is hydroxyl;
L¹ and L² are each independently pentanediyl, hexanediyl, heptanediyl, —(CH₂)₂O(CH₂)₂—, —(CH₂)₂O(CH₂)₃—, or —(CH₂)₂O(CH₂)₂O(CH₂)₂—; and
L³ is ethane-1,2-diyl, propane-1,3-diyl, butane-1,4-diyl, or pentane-1,5-diyl.

In still another embodiment, in Formula (III) or (IV),
R¹ is hydrogen, n-hexyl, n-octyl, 6,6,6-trifluorohex-1-yl, or 8,8,8-trifluorooct-1-yl;
R² is n-octyl, n-decyl, 8,8,8-trifluorooct-1-yl, or 10,10,10-trifluorodec-1-yl;
R³ is n-hexyl, n-octyl, 6,6,6-trifluorohex-1-yl, or 8,8,8-trifluorooct-1-yl;
R⁴ is n-octyl or 8,8,8-trifluorooct-1-yl;
R⁵ is hydroxyl;
L¹ and L² are each independently pentane-1,5-diyl, hexane-1,6-diyl, heptane-1,7-diyl, or —(CH₂)₂O(CH₂)₃—; and
L³ is ethane-1,2-diyl, propane-1,3-diyl, butane-1,4-diyl, or pentane-1,5-diyl.

In yet another embodiment, provided herein is a compound of Formula (V):

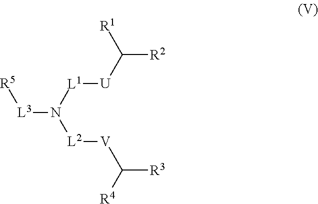

or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, or hydrate thereof; wherein U and V are each independently —C(O)—, —C(O)O—, or —OC(O)O—; and R¹, R², R³, R⁴, R⁵, L¹, L², and L³ are each as defined herein.

In one embodiment, in Formula (V),
R¹ is hydrogen or C₁₋₁₀ alkyl, optionally substituted with one or more substituents Q;
R², R³, and R⁴ are each independently C₁₋₁₀ alkyl, optionally substituted with one or more substituents Q;
R⁵ is hydroxyl;
L¹ and L² are each independently C₁₋₁₀ alkylene or C₁₋₁₀ heteroalkylene, each optionally substituted with one or more substituents Q;
L³ is C₁₋₁₀ alkylene, optionally substituted with one or more substituents Q; and
U and V are each independently —C(O)—, —C(O)O—, or —OC(O)O—.

In another embodiment, in Formula (V),
R¹ is hydrogen, unsubstituted C₁₋₁₀ alkyl, or C₁₋₁₀ alkyl substituted with one, two, or three substituents halo;
R², R³, and R⁴ are each independently unsubstituted C₁₋₁₀ alkyl, or C₁₋₁₀ alkyl substituted with one, two, or three substituents halo;
R⁵ is hydroxyl;
L¹ and L² are each independently unsubstituted C₁₋₁₀ alkylene or unsubstituted C₁₋₁₀ heteroalkylene;
L³ is unsubstituted C₁₋₁₀ alkylene; and
U and V are each independently —C(O)—, —C(O)O—, or —OC(O)O—.

In yet another embodiment, in Formula (V),
R¹ is hydrogen, hexyl, octyl, decyl, trifluorohexyl, trifluorooctyl, or trifluorodecyl;
R², R³, and R⁴ are each independently hexyl, octyl, decyl, trifluorohexyl, trifluorooctyl, or trifluorodecyl;
R⁵ is hydroxyl;
L¹ and L² are each independently pentanediyl, hexanediyl, heptanediyl, —(CH₂)₂O(CH₂)₂—, —(CH₂)₂O(CH₂)₃—, or —(CH₂)₂O(CH₂)₂O(CH₂)₂—;
L³ is ethanediyl, propanediyl, butanediyl, or pentanediyl; and
U and V are each independently —C(O)—, —C(O)O—, or —OC(O)O—.

In yet another embodiment, in Formula (V),
R¹ is hydrogen, hexyl, octyl, decyl, trifluorohexyl, trifluorooctyl, or trifluorodecyl;
R², R³, and R⁴ are each independently hexyl, octyl, decyl, trifluorohexyl, trifluorooctyl, or trifluorodecyl;
R⁵ is hydroxyl;
L¹ and L² are each independently pentanediyl, hexanediyl, heptanediyl, or —(CH₂)₂O(CH₂)₃—;
L³ is ethanediyl, propanediyl, butanediyl, or pentanediyl; and
U and V are each independently —C(O)—, —C(O)O—, or —OC(O)O—.

In yet another embodiment, in Formula (V),
R¹ is hydrogen, n-hexyl, n-octyl, n-decyl, 6,6,6-trifluorohex-1-yl, 8,8,8-trifluorooct-1-yl, or 10,10,10-trifluorodec-1-yl;
R², R³, and R⁴ are each independently n-hexyl, n-octyl, n-decyl, 6,6,6-trifluorohex-1-yl, 8,8,8-trifluorooct-1-yl, or 10,10,10-trifluorodec-1-yl;
R⁵ is hydroxyl;
L¹ and L² are each independently pentanediyl, hexanediyl, heptanediyl, —(CH₂)₂O(CH₂)₂—, —(CH₂)₂O(CH₂)₃—, or —(CH₂)₂O(CH₂)₂O(CH₂)₂—;
L³ is ethane-1,2-diyl, propane-1,3-diyl, butane-1,4-diyl, or pentane-1,5-diyl; and
U and V are each independently —C(O)—, —C(O)O—, or —OC(O)O—.

In yet another embodiment, in Formula (V),
R¹ is hydrogen, n-hexyl, n-octyl, n-decyl, 6,6,6-trifluorohex-1-yl, 8,8,8-trifluorooct-1-yl, or 10,10,10-trifluorodec-1-yl;
R², R³, and R⁴ are each independently n-hexyl, n-octyl, n-decyl, 6,6,6-trifluorohex-1-yl, 8,8,8-trifluorooct-1-yl, or 10,10,10-trifluorodec-1-yl;
R⁵ is hydroxyl;
L¹ and L² are each independently pentane-1,5-diyl, hexane-1,6-diyl, heptane-1,7-diyl, or —(CH₂)₂O(CH₂)₃—;
L³ is ethane-1,2-diyl, propane-1,3-diyl, butane-1,4-diyl, or pentane-1,5-diyl; and
U and V are each independently —C(O)—, —C(O)O—, or —OC(O)O—.

In yet another embodiment, in Formula (V),
R¹ is hydrogen, n-hexyl, n-octyl, 6,6,6-trifluorohex-1-yl, or 8,8,8-trifluorooct-1-yl;
R² is n-octyl, n-decyl, 8,8,8-trifluorooct-1-yl, or 10,10,10-trifluorodec-1-yl;
R³ is n-hexyl, n-octyl, 6,6,6-trifluorohex-1-yl, or 8,8,8-trifluorooct-1-yl;
R⁴ is n-octyl or 8,8,8-trifluorooct-1-yl;
R⁵ is hydroxyl;
L¹ and L² are each independently pentanediyl, hexanediyl, heptanediyl, —(CH₂)₂O(CH₂)₂—, —(CH₂)₂O(CH₂)₃—, or —(CH₂)₂O(CH₂)₂O(CH₂)₂—;
L³ is ethane-1,2-diyl, propane-1,3-diyl, butane-1,4-diyl, or pentane-1,5-diyl; and
U and V are each independently —C(O)—, —C(O)O—, or —OC(O)O—.

In still another embodiment, in Formula (V),
R¹ is hydrogen, n-hexyl, n-octyl, 6,6,6-trifluorohex-1-yl, or 8,8,8-trifluorooct-1-yl;
R² is n-octyl, n-decyl, 8,8,8-trifluorooct-1-yl, or 10,10,10-trifluorodec-1-yl;
R³ is n-hexyl, n-octyl, 6,6,6-trifluorohex-1-yl, or 8,8,8-trifluorooct-1-yl;
R⁴ is n-octyl or 8,8,8-trifluorooct-1-yl;
R⁵ is hydroxyl;
L¹ and L² are each independently pentane-1,5-diyl, hexane-1,6-diyl, heptane-1,7-diyl, or —(CH₂)₂O(CH₂)₃—;
L³ is ethane-1,2-diyl, propane-1,3-diyl, butane-1,4-diyl, or pentane-1,5-diyl; and
U and V are each independently —C(O)—, —C(O)O—, or —OC(O)O—.

In one embodiment, in Formula (V),
R¹ is hydrogen or C₁₋₁₀ alkyl, optionally substituted with one or more substituents Q;
R², R³, and R⁴ are each independently C₁₋₂₀ alkyl or C₁₋₂₀ heteroalkyl, optionally substituted with one or more substituents Q;
R⁵ is hydroxyl;
L¹ and L² are each independently C₁₋₁₀ alkylene or C₁₋₁₀ heteroalkylene, each optionally substituted with one or more substituents Q;
L³ is C₁₋₁₀ alkylene, optionally substituted with one or more substituents Q; and
U and V are each independently —C(O)—, —C(O)O—, or —OC(O)O—.

In another embodiment, in Formula (V),
R¹ is hydrogen, hexyl, octyl, decyl, trifluorohexyl, trifluorooctyl, or trifluorodecyl;
R², R³, and R⁴ are each independently hexyl, octyl, decyl, trifluorohexyl, trifluorooctyl, trifluorodecyl, —(CH₂)₂O(CH₂)₂O(CH₂)₄CH₃, or —(CH₂)₂O(CH₂)₂O(CH₂)₂O—(CH₂)₁₀CH₃;
R⁵ is hydroxyl;
L¹ and L² are each independently pentanediyl, hexanediyl, heptanediyl, —(CH₂)₂O(CH₂)₂—, —(CH₂)₂O(CH₂)₃—, —(CH₂)₂O(CH₂)₂O(CH₂)₂—, or —(CH₂)₆N(H)—;
L³ is ethanediyl, propanediyl, butanediyl, or pentanediyl; and
U and V are each independently —C(O)—, —C(O)O—, or —OC(O)O—.

In yet another embodiment, in Formula (V),
R¹ is hydrogen, n-hexyl, n-octyl, n-decyl, 6,6,6-trifluorohex-1-yl, 8,8,8-trifluorooct-1-yl, or 10,10,10-trifluorodec-1-yl;
R², R³, and R⁴ are each independently n-hexyl, n-octyl, n-decyl, 6,6,6-trifluorohex-1-yl, 8,8,8-trifluorooct-1-yl, 10,10,10-trifluorodec-1-yl, —(CH₂)₂O(CH₂)₂O(CH₂)₄—CH₃, or —(CH₂)₂O(CH₂)₂O(CH₂)₂O(CH₂)₁₀CH₃;
R⁵ is hydroxyl;
L¹ and L² are each independently pentane-1,5-diyl, hexane-1,6-diyl, heptane-1,7-diyl, —(CH₂)₂O(CH₂)₂—, —(CH₂)₂O(CH₂)₃—, —(CH₂)₂O(CH₂)₂O(CH₂)₂—, or —(CH₂)₆N(H)—;
L³ is ethane-1,2-diyl, propane-1,3-diyl, butane-1,4-diyl, or pentane-1,5-diyl; and
U and V are each independently —C(O)—, —C(O)O—, or —OC(O)O—.

In still another embodiment, in Formula (V),
R¹ is hydrogen, n-hexyl, n-octyl, 6,6,6-trifluorohex-1-yl, or 8,8,8-trifluorooct-1-yl;
R² is n-octyl, n-decyl, 8,8,8-trifluorooct-1-yl, 10,10,10-trifluorodec-1-yl, —(CH₂)₂O(CH₂)₂O(CH₂)₄CH₃, or —(CH₂)₂O(CH₂)₂O(CH₂)₂O(CH₂)₁₀CH₃;
R³ is n-hexyl, n-octyl, 6,6,6-trifluorohex-1-yl, or 8,8,8-trifluorooct-1-yl;
R⁴ is n-octyl or 8,8,8-trifluorooct-1-yl;
R⁵ is hydroxyl;
L¹ and L² are each independently pentane-1,5-diyl, hexane-1,6-diyl, heptane-1,7-diyl, —(CH₂)₂O(CH₂)₂—, —(CH₂)₂O(CH₂)₃—, —(CH₂)₂O(CH₂)₂O(CH₂)₂—, or —(CH₂)₆N(H)—;
L³ is ethane-1,2-diyl, propane-1,3-diyl, butane-1,4-diyl, or pentane-1,5-diyl; and
U and V are each independently —C(O)—, —C(O)O—, or —OC(O)O—.

In certain embodiments, in Formula (V), U and V are each —C(O)O—; and R¹, R², R³, or R⁴ is C₁₋₂₀ alkyl, substituted with one or more substituents Q. In certain embodiments, in Formula (V), U and V are each —C(O)O—; and R¹, R², R³, or R⁴ is C₁₋₂₀ alkyl, substituted with one or more halo. In certain embodiments, in Formula (V), U and V are each —C(O)O—; and R¹, R², R³, or R⁴ is C₁₋₂₀ alkyl, substituted with one or more fluoro. In certain embodiments, in Formula (V), U and V are each —C(O)O—; and R¹, R², R³, or R⁴ is C₁₋₂₀ alkyl, substituted with one, two, or three fluoro.

In certain embodiments, in Formula (V), U and V are each —C(O)O—; and L¹ or L² is C₁₋₂₀ heteroalkylene, optionally substituted with one or more substituents Q. In certain embodiments, in Formula (V), U and V are each —C(O)O—; and L¹ or L² is unsubstituted C₁₋₂₀ heteroalkylene. In certain embodiments, in Formula (V), U and V are each —C(O)O—; X and Z are each C(H); and L¹ and L² are each independently —NH—C₁₋₂₀ alkylene or —NH—C₁₋₂₀ hetero-alkylene, each optionally substituted with one or more substituents Q.

In certain embodiments, in Formula (V), U and V are each —C(O)O—; R¹, R², R³, or R⁴ is C₁₋₂₀ alkyl, substituted with one or more substituents Q; and L¹ or L² is C₁₋₂₀ heteroalkylene, optionally substituted with one or more substituents Q. In certain embodiments, in Formula (V), U and V are each —C(O)O—; R¹, R², R³, or R⁴ is C₁₋₂₀ alkyl, substituted with one or more halo; and L¹ or L² is unsubstituted C₁₋₂₀ heteroalkylene. In certain embodiments, in Formula (V), U and V are each —C(O)O—; R¹, R², R³, or R⁴ is C₁₋₂₀ alkyl, substituted with one or more fluoro; and L¹ or L² is unsubstituted C₁₋₂₀ heteroalkylene. In certain embodiments, in Formula (V), U and V are each —C(O)O—; R¹, R², R³, or R⁴ is C₁₋₂₀ alkyl, substituted with one, two, or three fluoro; and L¹ or L² is unsubstituted C₁₋₂₀ heteroalkylene.

In certain embodiments, in Formula (V), U and V are each —C(O)—; X and Z are each C(H); and L¹ and L² are each independently C₁₋₂₀ heteroalkylene, optionally substituted with one or more substituents Q. In certain embodiments, in Formula (V), U and V are each —C(O)—; X and Z are each C(H); and L¹ and L² are each independently unsubstituted C₁₋₂₀ heteroalkylene. In certain embodiments, in Formula (V), U and V are each —C(O)—; X and Z are each C(H); and L¹ and L² are each independently —NH—C₁₋₂₀ alkylene or —NH—C₁₋₂₀ hetero-alkylene, each optionally substituted with one or more substituents Q.

In yet another embodiment, provided herein is a compound of Formula (VI):

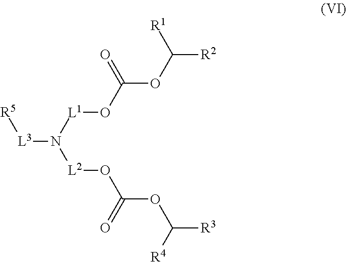

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof; wherein R¹, R², R³, R⁴, R⁵, L¹, L², and L³ are each as defined herein.

In yet another embodiment, provided herein is a compound of Formula (VII):

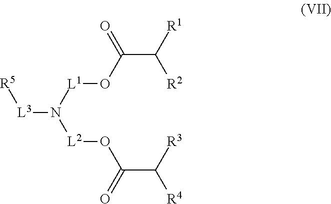

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof, wherein R¹, R², R³, R⁴, R⁵, L¹, L², and L³ are each as defined herein.

In yet another embodiment, provided herein is a compound of Formula (VIII):

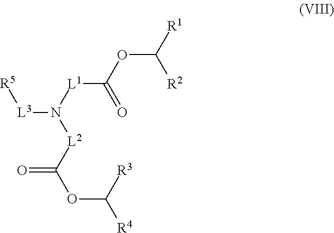

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or a hydrate thereof; wherein R¹, R², R³, R⁴, R⁵, L¹, L², and L³ are each as defined herein.

In one embodiment, in Formula (VI), (VII), or (VIII),
R¹ is hydrogen or C₁₋₁₀ alkyl, optionally substituted with one or more substituents Q;
R², R³, and R⁴ are each independently C₁₋₁₀ alkyl, optionally substituted with one or more substituents Q;
R⁵ is hydroxyl;
L¹ and L² are each independently C₁₋₁₀ alkylene or C₁₋₁₀ heteroalkylene, each optionally substituted with one or more substituents Q; and
L³ is C₁₋₁₀ alkylene, optionally substituted with one or more substituents Q.

In another embodiment, in Formula (VI), (VII), or (VIII),
R¹ is hydrogen, unsubstituted C₁₋₁₀ alkyl, or C₁₋₁₀ alkyl substituted with one, two, or three substituents halo;
R², R³, and R⁴ are each independently unsubstituted C₁₋₁₀ alkyl, or C₁₋₁₀ alkyl substituted with one, two, or three substituents halo;
R⁵ is hydroxyl;
L¹ and L² are each independently unsubstituted C₁₋₁₀ alkylene or unsubstituted C₁₋₁₀ heteroalkylene; and
L³ is unsubstituted C₁₋₁₀ alkylene.

In yet another embodiment, in Formula (VI), (VII), or (VIII),
R¹ is hydrogen, hexyl, octyl, decyl, trifluorohexyl, trifluorooctyl, or trifluorodecyl;
R², R³, and R⁴ are each independently hexyl, octyl, decyl, trifluorohexyl, trifluorooctyl, or trifluorodecyl;
R⁵ is hydroxyl;
L¹ and L² are each independently pentanediyl, hexanediyl, heptanediyl, —(CH₂)₂O(CH₂)₂—, —(CH₂)₂O(CH₂)₃—, or —(CH₂)₂O(CH₂)₂O(CH₂)₂—; and
L³ is ethanediyl, propanediyl, butanediyl, or pentanediyl.

In yet another embodiment, in Formula (VI), (VII), or (VIII),
R¹ is hydrogen, hexyl, octyl, decyl, trifluorohexyl, trifluorooctyl, or trifluorodecyl;
R², R³, and R⁴ are each independently hexyl, octyl, decyl, trifluorohexyl, trifluorooctyl, or trifluorodecyl;
R⁵ is hydroxyl;
L¹ and L² are each independently pentanediyl, hexanediyl, heptanediyl, or —(CH₂)₂O(CH₂)₃—; and
L³ is ethanediyl, propanediyl, butanediyl, or pentanediyl.

In yet another embodiment, in Formula (VI), (VII), or (VIII),
R¹ is hydrogen, n-hexyl, n-octyl, n-decyl, 6,6,6-trifluorohex-1-yl, 8,8,8-trifluorooct-1-yl, or 10,10,10-trifluorodec-1-yl;
R², R³, and R⁴ are each independently n-hexyl, n-octyl, n-decyl, 6,6,6-trifluorohex-1-yl, 8,8,8-trifluorooct-1-yl, or 10,10,10-trifluorodec-1-yl;
R⁵ is hydroxyl;
L¹ and L² are each independently pentane-1,5-diyl, hexane-1,6-diyl, heptane-1,7-diyl, —(CH₂)₂O(CH₂)₂—, —(CH₂)₂O(CH₂)₃—, or —(CH₂)₂O(CH₂)₂O(CH₂)₂—; and
L³ is ethane-1,2-diyl, propane-1,3-diyl, butane-1,4-diyl, or pentane-1,5-diyl.

In yet another embodiment, in Formula (VI), (VII), or (VIII),
R¹ is hydrogen, n-hexyl, n-octyl, n-decyl, 6,6,6-trifluorohex-1-yl, 8,8,8-trifluorooct-1-yl, or 10,10,10-trifluorodec-1-yl;
R², R³, and R⁴ are each independently n-hexyl, n-octyl, n-decyl, 6,6,6-trifluorohex-1-yl, 8,8,8-trifluorooct-1-yl, or 10,10,10-trifluorodec-1-yl;
R⁵ is hydroxyl;
L¹ and L² are each independently pentane-1,5-diyl, hexane-1,6-diyl, heptane-1,7-diyl, or —(CH₂)₂O(CH₂)₃—; and
L³ is ethane-1,2-diyl, propane-1,3-diyl, butane-1,4-diyl, or pentane-1,5-diyl.

In yet another embodiment, in Formula (VI), (VII), or (VIII),
R¹ is hydrogen, n-hexyl, n-octyl, 6,6,6-trifluorohex-1-yl, or 8,8,8-trifluorooct-1-yl;
R² is n-octyl, n-decyl, 8,8,8-trifluorooct-1-yl, or 10,10,10-trifluorodec-1-yl;
R³ is n-hexyl, n-octyl, 6,6,6-trifluorohex-1-yl, or 8,8,8-trifluorooct-1-yl;
R⁴ is n-octyl or 8,8,8-trifluorooct-1-yl;
R⁵ is hydroxyl;
L¹ and L² are each independently pentane-1,5-diyl, hexane-1,6-diyl, heptane-1,7-diyl, —(CH₂)₂O(CH₂)₂—, —(CH₂)₂O(CH₂)₃—, or —(CH₂)₂O(CH₂)₂O(CH₂)₂—; and
L³ is ethane-1,2-diyl, propane-1,3-diyl, butane-1,4-diyl, or pentane-1,5-diyl.

In still another embodiment, in Formula (VI), (VII), or (VIII),
R¹ is hydrogen, n-hexyl, n-octyl, 6,6,6-trifluorohex-1-yl, or 8,8,8-trifluorooct-1-yl;
R² is n-octyl, n-decyl, 8,8,8-trifluorooct-1-yl, or 10,10,10-trifluorodec-1-yl;
R³ is n-hexyl, n-octyl, 6,6,6-trifluorohex-1-yl, or 8,8,8-trifluorooct-1-yl;
R⁴ is n-octyl or 8,8,8-trifluorooct-1-yl;
R⁵ is hydroxyl;
L¹ and L² are each independently pentane-1,5-diyl, hexane-1,6-diyl, heptane-1,7-diyl, or —(CH₂)₂O(CH₂)₃—; and
L³ is ethane-1,2-diyl, propane-1,3-diyl, butane-1,4-diyl, or pentane-1,5-diyl.

In one embodiment, in Formula (VIII),
R¹ is hydrogen or C₁₋₁₀ alkyl, optionally substituted with one or more substituents Q;
R², R³, and R⁴ are each independently C₁₋₂₀ alkyl or C₁₋₂₀ heteroalkyl, optionally substituted with one or more substituents Q;
R⁵ is hydroxyl;
L¹ and L² are each independently C₁₋₁₀ alkylene or C₁₋₁₀ heteroalkylene, each optionally substituted with one or more substituents Q; and
L³ is C₁₋₁₀ alkylene, optionally substituted with one or more substituents Q.

In another embodiment, in Formula (VIII),
R¹ is hydrogen, hexyl, octyl, decyl, trifluorohexyl, trifluorooctyl, or trifluorodecyl;
R², R³, and R⁴ are each independently hexyl, octyl, decyl, trifluorohexyl, trifluorooctyl, trifluorodecyl, —(CH₂)₂O(CH₂)₂O(CH₂)₄CH₃, or —(CH₂)₂O(CH₂)₂O(CH₂)₂O—(CH₂)₁₀CH₃;
R⁵ is hydroxyl;
L¹ and L² are each independently pentanediyl, hexanediyl, heptanediyl, —(CH₂)₂O(CH₂)₂—, —(CH₂)₂O(CH₂)₃—, —(CH₂)₂O(CH₂)₂O(CH₂)₂—, or —(CH₂)₆N(H)—; and
L³ is ethanediyl, propanediyl, butanediyl, or pentanediyl.

In yet another embodiment, in Formula (VIII),
R¹ is hydrogen, n-hexyl, n-octyl, n-decyl, 6,6,6-trifluorohex-1-yl, 8,8,8-trifluorooct-1-yl, or 10,10,10-trifluorodec-1-yl;
R², R³, and R⁴ are each independently n-hexyl, n-octyl, n-decyl, 6,6,6-trifluorohex-1-yl, 8,8,8-trifluorooct-1-yl, 10,10,10-trifluorodec-1-yl, —(CH₂)₂O(CH₂)₂O(CH₂)₄—CH₃, or —(CH₂)₂O(CH₂)₂O(CH₂)₂O(CH₂)₁₀CH₃;
R⁵ is hydroxyl;
L¹ and L² are each independently pentane-1,5-diyl, hexane-1,6-diyl, heptane-1,7-diyl, —(CH₂)₂O(CH₂)₂—, —(CH₂)₂O(CH₂)₃—, —(CH₂)₂O(CH₂)₂O(CH₂)₂—, or —(CH₂)₆N(H)—; and
L³ is ethane-1,2-diyl, propane-1,3-diyl, butane-1,4-diyl, or pentane-1,5-diyl.

In still another embodiment, in Formula (VIII),
R¹ is hydrogen, n-hexyl, n-octyl, 6,6,6-trifluorohex-1-yl, or 8,8,8-trifluorooct-1-yl;
R² is n-octyl, n-decyl, 8,8,8-trifluorooct-1-yl, 10,10,10-trifluorodec-1-yl, —(CH₂)₂O(CH₂)₂O(CH₂)₄CH₃, or —(CH₂)₂O(CH₂)₂O(CH₂)₂O(CH₂)₁₀CH₃;
R³ is n-hexyl, n-octyl, 6,6,6-trifluorohex-1-yl, or 8,8,8-trifluorooct-1-yl;
R⁴ is n-octyl or 8,8,8-trifluorooct-1-yl;
R⁵ is hydroxyl;
L¹ and L² are each independently pentane-1,5-diyl, hexane-1,6-diyl, heptane-1,7-diyl, —(CH₂)₂O(CH₂)₂—, —(CH₂)₂O(CH₂)₃—, —(CH₂)₂O(CH₂)₂O(CH₂)₂—, or —(CH₂)₆N(H)—; and
L³ is ethane-1,2-diyl, propane-1,3-diyl, butane-1,4-diyl, or pentane-1,5-diyl.

In certain embodiments, in Formula (VI), (VII), or (VIII), R¹, R², R³, or R⁴ is C₁₋₂₀ alkyl, substituted with one or more substituents Q. In certain embodiments, in Formula (VI), (VII), or (VIII), R¹, R², R³, or R⁴ is C₁₋₂₀ alkyl, substituted with one or more halo. In certain embodiments, in Formula (VI), (VII), or (VIII), R¹, R², R³, or R⁴ is C₁₋₂₀ alkyl, substituted with one or more fluoro. In certain embodiments, in Formula (VI), (VII), or (VIII), R¹, R², R³, or R⁴ is C₁₋₂₀ alkyl, substituted with one, two, or three fluoro.

In certain embodiments, in Formula (VI), (VII), or (VIII), L¹ or L² is C₁₋₂₀ heteroalkylene, optionally substituted with one or more substituents Q. In certain embodiments, in Formula (VI), (VII), or (VIII), L¹ or L² is unsubstituted C₁₋₂₀ heteroalkylene. In certain embodiments, in Formula (VIII), L¹ and L² are each independently —NH—C₁₋₂₀ alkylene or —NH—C₁₋₂₀ heteroalkylene, each optionally substituted with one or more substituents Q.

In certain embodiments, in Formula (VI), (VII), or (VIII), R¹, R², R³, or R⁴ is C₁₋₂₀ alkyl, substituted with one or more substituents Q; and L¹ or L² is C₁₋₂₀ heteroalkylene, optionally substituted with one or more substituents Q. In certain embodiments, in Formula (VI), (VII), or (VIII), R¹, R², R³, or R⁴ is C₁₋₂₀ alkyl, substituted with one or more halo; and L¹ or L² is unsubstituted C₁₋₂₀ heteroalkylene. In certain embodiments, in Formula (VI), (VII), or (VIII), R¹, R², R³, or R⁴ is C₁₋₂₀ alkyl, substituted with one or more fluoro; and L¹ or L² is unsubstituted C₁₋₂₀ heteroalkylene. In certain embodiments, in Formula (VI), (VII), or (VIII), R¹, R², R³, or R⁴ is C₁₋₂₀ alkyl, substituted with one, two, or three fluoro; and L¹ or L² is unsubstituted C₁₋₂₀ heteroalkylene.

In yet another embodiment, provided herein is a compound of Formula (IX):

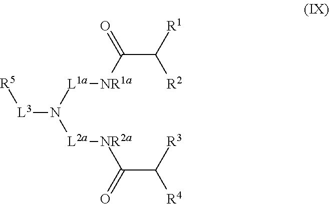

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or a hydrate thereof; wherein:
L^{1a} and L^{2a} are each independently C₁₋₂₀ alkylene or C₁₋₂₀ heteroalkylene, each optionally substituted with one or more substituents Q;
R^{1a} and R^{2a} are each independently (i) hydrogen; or (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each optionally substituted with one or more substituents Q; and
R¹, R², R³, R⁴, R⁵, and L³ are each as defined herein.

In still another embodiment, provided herein is a compound of Formula (X):

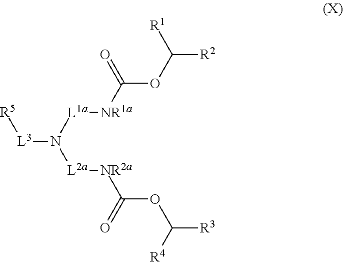

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or a hydrate thereof; wherein R¹, R², R³, R⁴, R⁵, R^{1a}, R^{2a}, L³, L^{1a}, and L^{2a} are each as defined herein.

In one embodiment, in Formula (IX) or (X),
R¹ is hydrogen or C₁₋₁₀ alkyl, optionally substituted with one or more substituents Q;
R², R³, and R⁴ are each independently C₁₋₂₀ alkyl or C₁₋₂₀ heteroalkyl, each optionally substituted with one or more substituents Q;
R⁵ is hydroxyl;
R^{1a} and R^{2a} are each independently hydrogen or C₁₋₁₀ alkyl optionally substituted with one or more substituents Q;
L^{1a} and L^{2a} are each independently C₁₋₁₀ alkylene or C₁₋₁₀ heteroalkylene, each optionally substituted with one or more substituents Q; and
L³ is C₁₋₁₀ alkylene, optionally substituted with one or more substituents Q.

In another embodiment, in Formula (IX) or (X),
R¹ is hydrogen, hexyl, octyl, decyl, trifluorohexyl, trifluorooctyl, or trifluorodecyl;
R², R³, and R⁴ are each independently hexyl, octyl, decyl, trifluorohexyl, trifluorooctyl, trifluorodecyl, —(CH₂)₂O(CH₂)₂O(CH₂)₄CH₃, or —(CH₂)₂O(CH₂)₂O(CH₂)₂O—(CH₂)₁₀CH₃;
R⁵ is hydroxyl;
R^{1a} and R^{2a} are each hydrogen;
L^{1a} and L^{2a} are each independently pentanediyl, hexanediyl, or heptanediyl; and
L³ is ethanediyl, propanediyl, butanediyl, or pentanediyl.

In yet another embodiment, in Formula (IX) or (X),
R¹ is hydrogen, n-hexyl, n-octyl, n-decyl, 6,6,6-trifluorohex-1-yl, 8,8,8-trifluorooct-1-yl, or 10,10,10-trifluorodec-1-yl;
R², R³, and R⁴ are each independently n-hexyl, n-octyl, n-decyl, 6,6,6-trifluorohex-1-yl, 8,8,8-trifluorooct-1-yl, 10,10,10-trifluorodec-1-yl, —(CH₂)₂O(CH₂)₂O(CH₂)₄—CH₃, or —(CH₂)₂O(CH₂)₂O(CH₂)₂O(CH₂)₁₀CH₃;
R⁵ is hydroxyl;
R^{1a} and R^{2a} are each hydrogen;
L^{1a} and L^{2a} are each independently pentanediyl, hexanediyl, or heptanediyl; and
L³ is ethane-1,2-diyl, propane-1,3-diyl, butane-1,4-diyl, or pentane-1,5-diyl.

In still another embodiment, in Formula (IX) or (X),
R¹ is hydrogen, n-hexyl, n-octyl, 6,6,6-trifluorohex-1-yl, or 8,8,8-trifluorooct-1-yl;
R² is n-octyl, n-decyl, 8,8,8-trifluorooct-1-yl, 10,10,10-trifluorodec-1-yl, —(CH₂)₂O(CH₂)₂O(CH₂)₄CH₃, or —(CH₂)₂O(CH₂)₂O(CH₂)₂O(CH₂)₁₀CH₃;
R³ is n-hexyl, n-octyl, 6,6,6-trifluorohex-1-yl, or 8,8,8-trifluorooct-1-yl;
R⁴ is n-octyl or 8,8,8-trifluorooct-1-yl;
R⁵ is hydroxyl;
R^{1a} and R^{2a} are each hydrogen;
L^{1a} and L^{2a} are each independently pentanediyl, hexanediyl, or heptanediyl; and
L³ is ethane-1,2-diyl, propane-1,3-diyl, butane-1,4-diyl, or pentane-1,5-diyl.

The groups, R¹, R², R³, R⁴, R⁵, R^{1a}, R^{2a}, L¹, L², L³, L^{1a}, L^{2a}, U, V, X, and Z, in formulae described herein, including Formulae (I) to (X), are further defined in the embodiments described herein. All combinations of the embodiments provided herein for such groups are within the scope of this disclosure.

In certain embodiments, R¹ is hydrogen. In certain embodiments, R¹ is C₁₋₂₀ alkyl, optionally substituted with one or more substituents Q. In certain embodiments, R¹ is unsubstituted C₁₋₂₀ alkyl. In certain embodiments, R¹ is C₁₋₂₀ alkyl, substituted with one or more substituents Q. In certain embodiments, R¹ is C₁₋₂₀ alkyl, substituted with one or more halo. In certain embodiments, R¹ is C₁₋₂₀ alkyl, substituted with one or more fluoro. In certain embodiments, R¹ is C₁₋₂₀ alkyl, substituted with one, two, or three fluoro.

In certain embodiments, R¹ is hydrogen. In certain embodiments, R¹ is C₁₋₁₀ alkyl, optionally substituted with one or more substituents Q. In certain embodiments, R¹ is unsubstituted C₁₋₁₀ alkyl. In certain embodiments, R¹ is C₁₋₁₀ alkyl, substituted with one or more substituents Q. In certain embodiments, R¹ is C₁—.1 alkyl, substituted with one or more halo. In certain embodiments, R¹ is C₁₋₁₀ alkyl, substituted with one or more fluoro. In certain embodiments, R¹ is C₁₋₁₀ alkyl, substituted with one, two, or three fluoro.

In certain embodiments, R¹ is methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, or eicosyl, each optionally substituted with one or more substituents Q. In certain embodiments, R¹ is methyl, ethyl, propyl, butyl, pentyl, hexyl, heptyl, octyl, nonyl, or decyl, each optionally substituted with one or more substituents Q. In certain embodiments, R¹ is butyl, pentyl, hexyl, heptyl, octyl, nonyl, or decyl, each optionally substituted with one or more substituents Q.

In certain embodiments, R¹ is methyl, ethyl, n-propyl, n-butyl, n-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl, or n-eicosyl, each optionally substituted with one or more substituents Q. In certain embodiments, R¹ is methyl, ethyl, n-propyl, n-butyl, n-pentyl, n-hexyl, n-heptyl, n-octyl, n-nonyl, or n-decyl, each optionally substituted with one or more substituents Q. In certain embodiments, R¹ is n-butyl, n-pentyl, n-hexyl, n-heptyl, n-octyl, n-nonyl, or n-decyl, each optionally substituted with one or more substituents Q.

In certain embodiments, R¹ is methyl, ethyl, n-propyl, n-butyl, n-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl, or n-eicosyl, each of which is unsubstituted. In certain embodiments, R¹ is methyl, ethyl, n-propyl, n-butyl, n-pentyl, n-hexyl, n-heptyl, n-octyl, n-nonyl, or n-decyl, each of which is unsubstituted. In certain embodiments, R¹ is n-butyl, n-pentyl, n-hexyl, n-heptyl, n-octyl, n-nonyl, or n-decyl, each of which is unsubstituted.

In certain embodiments, R¹ is trifluoromethyl, trifluoroethyl, trifluoropropyl, trifluorobutyl, trifluoropentyl, trifluorohexyl, trifluoroheptyl, trifluorooctyl, trifluorononyl, trifluorodecyl, trifluoroundecyl, trifluorododecyl, trifluorotridecyl, trifluorotetradecyl, trifluoro-pentadecyl, trifluorohexadecyl, trifluoroheptadecyl, trifluorooctadecyl, trifluorononadecyl, or trifluoroeicosyl. In certain embodiments, R¹ is trifluoromethyl, trifluoroethyl, trifluoropropyl, trifluorobutyl, trifluoropentyl, trifluorohexyl, trifluoroheptyl, trifluorooctyl, trifluorononyl, or trifluorodecyl. In certain embodiments, R¹ is trifluorobutyl, trifluoropentyl, trifluorohexyl, trifluoroheptyl, trifluorooctyl, trifluorononyl, or trifluorodecyl.

In certain embodiments, R¹ is trifluoromethyl, 2,2,2-trifluoroethyl, 3,3,3-trifluoro-prop-1-yl, 4,4,4-trifluorobut-1-yl, 5,5,5-trifluoropent-1-yl, 6,6,6-trifluorohex-1-yl, 7,7,7-trifluorohept-1-yl, 8,8,8-trifluorooct-1-yl, 9,9,9-trifluoronon-1-yl, 10,10,10-trifluorodec-1-yl, 11,11,11-trifluoroundec-1-yl, 12,12,12-trifluorododec-1-yl, 13,13,13-trifluorotridec-1-yl, 14,14,14-trifluorotetradec-1-yl, 15,15,15-trifluoropentadec-1-yl, 16,16,16-trifluorohexadec-1-yl, 17,17,17-trifluoroheptadec-1-yl, 18,18,18-trifluorooctadec-1-yl, 19,19,19-trifluorononadec-1-yl, or 20,20,20-trifluoroeicosyl. In certain embodiments, R¹ is trifluoromethyl, 2,2,2-trifluoroethyl, 3,3,3-trifluoroprop-1-yl, 4,4,4-trifluorobut-1-yl, 5,5,5-trifluoropent-1-yl, 6,6,6-trifluorohex-1-yl, 7,7,7-trifluorohept-1-yl, 8,8,8-trifluorooct-1-yl, 9,9,9-trifluoronon-1-yl, or 10,10,10-trifluorodec-1-yl. In certain embodiments, R¹ is 4,4,4-trifluorobut-1-yl, 5,5,5-trifluoropent-1-yl, 6,6,6-trifluorohex-1-yl, 7,7,7-trifluorohept-1-yl, 8,8,8-trifluorooct-1-yl, 9,9,9-trifluoronon-1-yl, or 10,10,10-trifluorodec-1-yl.

In certain embodiments, R¹ is C₁₋₂₀ heteroalkyl, optionally substituted with one or more substituents Q. In certain embodiments, R¹ is 2-(2-(pentyloxy)ethoxy)ethyl or 2-(2-(2-(undecyloxy)ethoxy)ethoxy)ethyl. In certain embodiments, R¹ is C₂₋₂₀ alkenyl, optionally substituted with one or more substituents Q. In certain embodiments, R¹ is C₂₋₂₀ alkynyl, optionally substituted with one or more substituents Q.

In certain embodiments, R¹ is hydrogen, hexyl, octyl, decyl, trifluorohexyl, trifluorooctyl, or trifluorodecyl. In certain embodiments, R¹ is hydrogen, n-hexyl, n-octyl, n-decyl, 6,6,6-trifluorohex-1-yl, 8,8,8-trifluorooct-1-yl, or 10,10,10-trifluorodec-1-yl. In certain embodiments, R¹ is hydrogen, n-hexyl, n-octyl, 6,6,6-trifluorohex-1-yl, or 8,8,8-trifluorooct-1-yl. In certain embodiments, R¹ is hydrogen, n-octyl, n-decyl, 8,8,8-trifluorooct-1-yl, or 10,10,10-trifluorodec-1-yl. In certain embodiments, R¹ is hydrogen, n-hexyl, n-octyl, n-decyl, 6,6,6-trifluorohex-1-yl, 8,8,8-trifluorooct-1-yl, 10,10,10-trifluorodec-1-yl, 2-(2-(pentyloxy)ethoxy)-ethyl, or 2-(2-(2-(undecyloxy)ethoxy)ethoxy)ethyl. In certain embodiments, R¹ is hydrogen, n-hexyl, n-octyl, 6,6,6-trifluorohex-1-yl, 8,8,8-trifluorooct-1-yl, 2-(2-(pentyloxy)ethoxy)-ethyl, or 2-(2-(2-(undecyloxy)ethoxy)ethoxy)ethyl. In certain embodiments, R¹ is hydrogen, n-octyl, n-decyl, 8,8,8-trifluorooct-1-yl, 10,10,10-trifluorodec-1-yl, 2-(2-(pentyloxy)ethoxy)-ethyl, or 2-(2-(2-(undecyloxy)ethoxy)ethoxy)ethyl.

In certain embodiments, R² is C₁₋₂₀ alkyl, optionally substituted with one or more substituents Q. In certain embodiments, R² is unsubstituted C₁₋₂₀ alkyl. In certain embodiments, R² is C₁₋₂₀ alkyl, substituted with one or more substituents Q. In certain embodiments, R² is C₁₋₂₀ alkyl, substituted with one or more halo. In certain embodiments, R² is C₁₋₂₀ alkyl, substituted with one or more fluoro. In certain embodiments, R² is C₁₋₂₀ alkyl, substituted with one, two, or three fluoro.

In certain embodiments, R² is C₁₋₁₀ alkyl, optionally substituted with one or more substituents Q. In certain embodiments, R² is unsubstituted C₁₋₁₀ alkyl. In certain embodiments, R² is C₁₋₁₀ alkyl, substituted with one or more substituents Q. In certain embodiments, R² is C₁₋₁₀ alkyl, substituted with one or more halo. In certain embodiments, R² is C₁₋₁₀ alkyl, substituted with one or more fluoro. In certain embodiments, R² is C₁₋₁₀ alkyl, substituted with one, two, or three fluoro.

In certain embodiments, R² is methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, or eicosyl, each optionally substituted with one or more substituents Q. In certain embodiments, R² is methyl, ethyl, propyl, butyl, pentyl, hexyl, heptyl, octyl, nonyl, or decyl, each optionally substituted with one or more substituents Q. In certain embodiments, R² is butyl, pentyl, hexyl, heptyl, octyl, nonyl, or decyl, each optionally substituted with one or more substituents Q.

In certain embodiments, R² is methyl, ethyl, n-propyl, n-butyl, n-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl, or n-eicosyl, each optionally substituted with one or more substituents Q. In certain embodiments, R² is methyl, ethyl, n-propyl, n-butyl, n-pentyl, n-hexyl, n-heptyl, n-octyl, n-nonyl, or n-decyl, each optionally substituted with one or more substituents Q. In certain embodiments, R² is n-butyl, n-pentyl, n-hexyl, n-heptyl, n-octyl, n-nonyl, or n-decyl, each optionally substituted with one or more substituents Q.

In certain embodiments, R² is methyl, ethyl, n-propyl, n-butyl, n-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl, or n-eicosyl, each of which is unsubstituted. In certain embodiments, R² is methyl, ethyl, n-propyl, n-butyl, n-pentyl, n-hexyl, n-heptyl, n-octyl, n-nonyl, or n-decyl, each of which is unsubstituted. In certain embodiments, R² is n-butyl, n-pentyl, n-hexyl, n-heptyl, n-octyl, n-nonyl, or n-decyl, each of which is unsubstituted.

In certain embodiments, R² is trifluoromethyl, trifluoroethyl, trifluoropropyl, trifluorobutyl, trifluoropentyl, trifluorohexyl, trifluoroheptyl, trifluorooctyl, trifluorononyl, trifluorodecyl, trifluoroundecyl, trifluorododecyl, trifluorotridecyl, trifluorotetradecyl, trifluoro-pentadecyl, trifluorohexadecyl, trifluoroheptadecyl, trifluorooctadecyl, trifluorononadecyl, or trifluoroeicosyl. In certain embodiments, R² is trifluoromethyl, trifluoroethyl, trifluoropropyl, trifluorobutyl, trifluoropentyl, trifluorohexyl, trifluoroheptyl, trifluorooctyl, trifluorononyl, or trifluorodecyl. In certain embodiments, R² is trifluorobutyl, trifluoropentyl, trifluorohexyl, trifluoroheptyl, trifluorooctyl, trifluorononyl, or trifluorodecyl.

In certain embodiments, R² is trifluoromethyl, 2,2,2-trifluoroethyl, 3,3,3-trifluoro-prop-1-yl, 4,4,4-trifluorobut-1-yl, 5,5,5-trifluoropent-1-yl, 6,6,6-trifluorohex-1-yl, 7,7,7-trifluorohept-1-yl, 8,8,8-trifluorooct-1-yl, 9,9,9-trifluoronon-1-yl, 10,10,10-trifluorodec-1-yl, 11,11,11-trifluoroundec-1-yl, 12,12,12-trifluorododec-1-yl, 13,13,13-trifluorotridec-1-yl, 14,14,14-trifluorotetradec-1-yl, 15,15,15-trifluoropentadec-1-yl, 16,16,16-trifluorohexadec-1-yl, 17,17,17-trifluoroheptadec-1-yl, 18,18,18-trifluorooctadec-1-yl, 19,19,19-trifluorononadec-1-yl, or 20,20,20-trifluoroeicosyl. In certain embodiments, R² is trifluoromethyl, 2,2,2-trifluoroethyl, 3,3,3-trifluoroprop-1-yl, 4,4,4-trifluorobut-1-yl, 5,5,5-trifluoropent-1-yl, 6,6,6-trifluorohex-1-yl, 7,7,7-trifluorohept-1-yl, 8,8,8-trifluorooct-1-yl, 9,9,9-trifluoronon-1-yl, or 10,10,10-trifluorodec-1-yl. In certain embodiments, R² is 4,4,4-trifluorobut-1-yl, 5,5,5-trifluoropent-1-yl, 6,6,6-trifluorohex-1-yl, 7,7,7-trifluorohept-1-yl, 8,8,8-trifluorooct-1-yl, 9,9,9-trifluoronon-1-yl, or 10,10,10-trifluorodec-1-yl.

In certain embodiments, R² is C₁₋₂₀ heteroalkyl, optionally substituted with one or more substituents Q. In certain embodiments, R² is 2-(2-(pentyloxy)ethoxy)ethyl or 2-(2-(2-(undecyloxy)ethoxy)ethoxy)ethyl. In certain embodiments, R² is C₂₋₂₀ alkenyl, optionally substituted with one or more substituents Q. In certain embodiments, R² is C₂₋₂₀ alkynyl, optionally substituted with one or more substituents Q.

In certain embodiments, R² is hexyl, octyl, decyl, trifluorohexyl, trifluorooctyl, or trifluorodecyl. In certain embodiments, R² is n-hexyl, n-octyl, n-decyl, 6,6,6-trifluorohex-1-yl, 8,8,8-trifluorooct-1-yl, or 10,10,10-trifluorodec-1-yl. In certain embodiments, R² is n-octyl, n-decyl, 8,8,8-trifluorooct-1-yl, or 10,10,10-trifluorodec-1-yl. In certain embodiments, R² is n-hexyl, n-octyl, n-decyl, 6,6,6-trifluorohex-1-yl, 8,8,8-trifluorooct-1-yl, 10,10,10-trifluorodec-1-yl, 2-(2-(pentyloxy)ethoxy)ethyl, or 2-(2-(2-(undecyloxy)ethoxy)ethoxy)ethyl. In certain embodiments, R² is n-octyl, n-decyl, 8,8,8-trifluorooct-1-yl, 10,10,10-trifluorodec-1-yl, 2-(2-(pentyloxy)ethoxy)ethyl, or 2-(2-(2-(undecyloxy)ethoxy)ethoxy)ethyl.

In certain embodiments, R³ is C₁₋₂₀ alkyl, optionally substituted with one or more substituents Q. In certain embodiments, R³ is unsubstituted C₁₋₂₀ alkyl. In certain embodiments, R³ is C₁₋₂₀ alkyl, substituted with one or more substituents Q. In certain embodiments, R³ is C₁₋₂₀ alkyl, substituted with one or more halo. In certain embodiments, R³ is C₁₋₂₀ alkyl, substituted with one or more fluoro. In certain embodiments, R³ is C₁₋₂₀ alkyl, substituted with one, two, or three fluoro.

In certain embodiments, R³ is C₁₋₁₀ alkyl, optionally substituted with one or more substituents Q. In certain embodiments, R³ is unsubstituted C₁₋₁₀ alkyl. In certain embodiments, R³ is C₁₋₁₀ alkyl, substituted with one or more substituents Q. In certain embodiments, R³ is C₁₋₁₀ alkyl, substituted with one or more halo. In certain embodiments, R³ is C₁₋₁₀ alkyl, substituted with one or more fluoro. In certain embodiments, R³ is C₁₋₁₀ alkyl, substituted with one, two, or three fluoro.

In certain embodiments, R³ is methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, or eicosyl, each optionally substituted with one or more substituents Q. In certain embodiments, R³ is methyl, ethyl, propyl, butyl, pentyl, hexyl, heptyl, octyl, nonyl, or decyl, each optionally substituted with one or more substituents Q. In certain embodiments, R³ is butyl, pentyl, hexyl, heptyl, octyl, nonyl, or decyl, each optionally substituted with one or more substituents Q.

In certain embodiments, R³ is methyl, ethyl, n-propyl, n-butyl, n-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl, or n-eicosyl, each optionally substituted with one or more substituents Q. In certain embodiments, R³ is methyl, ethyl, n-propyl, n-butyl, n-pentyl, n-hexyl, n-heptyl, n-octyl, n-nonyl, or n-decyl, each optionally substituted with one or more substituents Q. In certain embodiments, R³ is n-butyl, n-pentyl, n-hexyl, n-heptyl, n-octyl, n-nonyl, or n-decyl, each optionally substituted with one or more substituents Q.

In certain embodiments, R³ is methyl, ethyl, n-propyl, n-butyl, n-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl, or n-eicosyl, each of which is unsubstituted. In certain embodiments, R³ is methyl, ethyl, n-propyl, n-butyl, n-pentyl, n-hexyl, n-heptyl, n-octyl, n-nonyl, or n-decyl, each of which is unsubstituted. In certain embodiments, R³ is n-butyl, n-pentyl, n-hexyl, n-heptyl, n-octyl, n-nonyl, or n-decyl, each of which is unsubstituted.

In certain embodiments, R³ is trifluoromethyl, trifluoroethyl, trifluoropropyl, trifluorobutyl, trifluoropentyl, trifluorohexyl, trifluoroheptyl, trifluorooctyl, trifluorononyl, trifluorodecyl, trifluoroundecyl, trifluorododecyl, trifluorotridecyl, trifluorotetradecyl, trifluoro-pentadecyl, trifluorohexadecyl, trifluoroheptadecyl, trifluorooctadecyl, trifluorononadecyl, or trifluoroeicosyl. In certain embodiments, R³ is trifluoromethyl, trifluoroethyl, trifluoropropyl, trifluorobutyl, trifluoropentyl, trifluorohexyl, trifluoroheptyl, trifluorooctyl, trifluorononyl, or trifluorodecyl. In certain embodiments, R³ is trifluorobutyl, trifluoropentyl, trifluorohexyl, trifluoroheptyl, trifluorooctyl, trifluorononyl, or trifluorodecyl.

In certain embodiments, R³ is trifluoromethyl, 2,2,2-trifluoroethyl, 3,3,3-trifluoro-prop-1-yl, 4,4,4-trifluorobut-1-yl, 5,5,5-trifluoropent-1-yl, 6,6,6-trifluorohex-1-yl, 7,7,7-trifluorohept-1-yl, 8,8,8-trifluorooct-1-yl, 9,9,9-trifluoronon-1-yl, 10,10,10-trifluorodec-1-yl, 11,11,11-trifluoroundec-1-yl, 12,12,12-trifluorododec-1-yl, 13,13,13-trifluorotridec-1-yl, 14,14,14-trifluorotetradec-1-yl, 15,15,15-trifluoropentadec-1-yl, 16,16,16-trifluorohexadec-1-yl, 17,17,17-trifluoroheptadec-1-yl, 18,18,18-trifluorooctadec-1-yl, 19,19,19-trifluorononadec-1-yl, or 20,20,20-trifluoroeicosyl. In certain embodiments, R³ is trifluoromethyl, 2,2,2-trifluoroethyl, 3,3,3-trifluoroprop-1-yl, 4,4,4-trifluorobut-1-yl, 5,5,5-trifluoropent-1-yl, 6,6,6-trifluorohex-1-yl, 7,7,7-trifluorohept-1-yl, 8,8,8-trifluorooct-1-yl, 9,9,9-trifluoronon-1-yl, or 10,10,10-trifluorodec-1-yl. In certain embodiments, R³ is 4,4,4-trifluorobut-1-yl, 5,5,5-trifluoropent-1-yl, 6,6,6-trifluorohex-1-yl, 7,7,7-trifluorohept-1-yl, 8,8,8-trifluorooct-1-yl, 9,9,9-trifluoronon-1-yl, or 10,10,10-trifluorodec-1-yl.

In certain embodiments, R³ is C₁₋₂₀ heteroalkyl, optionally substituted with one or more substituents Q. In certain embodiments, R³ is 2-(2-(pentyloxy)ethoxy)ethyl or 2-(2-(2-(undecyloxy)ethoxy)ethoxy)ethyl. In certain embodiments, R³ is C₂₋₂₀ alkenyl, optionally substituted with one or more substituents Q. In certain embodiments, R³ is C₂₋₂₀ alkynyl, optionally substituted with one or more substituents Q.

In certain embodiments, R³ is hexyl, octyl, decyl, trifluorohexyl, trifluorooctyl, or trifluorodecyl. In certain embodiments, R³ is n-hexyl, n-octyl, n-decyl, 6,6,6-trifluorohex-1-yl, 8,8,8-trifluorooct-1-yl, or 10,10,10-trifluorodec-1-yl. In certain embodiments, R³ is n-hexyl, n-octyl, 6,6,6-trifluorohex-1-yl, or 8,8,8-trifluorooct-1-yl. In certain embodiments, R³ is n-hexyl, n-octyl, n-decyl, 6,6,6-trifluorohex-1-yl, 8,8,8-trifluorooct-1-yl, 10,10,10-trifluorodec-1-yl, 2-(2-(pentyloxy)ethoxy)ethyl, or 2-(2-(2-(undecyloxy)ethoxy)ethoxy)ethyl. In certain embodiments, R³ is n-hexyl, n-octyl, 6,6,6-trifluorohex-1-yl, 8,8,8-trifluorooct-1-yl, 2-(2-(pentyloxy)ethoxy)-ethyl, or 2-(2-(2-(undecyloxy)ethoxy)ethoxy)ethyl.

In certain embodiments, R⁴ is C₁₋₂₀ alkyl, optionally substituted with one or more substituents Q. In certain embodiments, R⁴ is unsubstituted C₁₋₂₀ alkyl. In certain embodiments, R⁴ is C₁₋₂₀ alkyl, substituted with one or more substituents Q. In certain embodiments, R⁴ is C₁₋₂₀ alkyl, substituted with one or more halo. In certain embodiments, R⁴ is C₁₋₂₀ alkyl, substituted with one or more fluoro. In certain embodiments, R⁴ is C₁₋₂₀ alkyl, substituted with one, two, or three fluoro.

In certain embodiments, R⁴ is C₁₋₁₀ alkyl, optionally substituted with one or more substituents Q. In certain embodiments, R⁴ is unsubstituted C₁₋₁₀ alkyl. In certain embodiments, R⁴ is C₁₋₁₀ alkyl, substituted with one or more substituents Q. In certain embodiments, R⁴ is C₁₋₁₀ alkyl, substituted with one or more halo. In certain embodiments, R⁴ is C₁₋₁₀ alkyl, substituted with one or more fluoro. In certain embodiments, R⁴ is C₁₋₁₀ alkyl, substituted with one, two, or three fluoro.

In certain embodiments, R⁴ is methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, or eicosyl, each optionally substituted with one or more substituents Q. In certain embodiments, R⁴ is methyl, ethyl, propyl, butyl, pentyl, hexyl, heptyl, octyl, nonyl, or decyl, each optionally substituted with one or more substituents Q. In certain embodiments, R⁴ is butyl, pentyl, hexyl, heptyl, octyl, nonyl, or decyl, each optionally substituted with one or more substituents Q.

In certain embodiments, R⁴ is methyl, ethyl, n-propyl, n-butyl, n-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl, or n-eicosyl, each optionally substituted with one or more substituents Q. In certain embodiments, R⁴ is methyl, ethyl, n-propyl, n-butyl, n-pentyl, n-hexyl, n-heptyl, n-octyl, n-nonyl, or n-decyl, each optionally substituted with one or more substituents Q. In certain embodiments, R⁴ is n-butyl, n-pentyl, n-hexyl, n-heptyl, n-octyl, n-nonyl, or n-decyl, each optionally substituted with one or more substituents Q.

In certain embodiments, R⁴ is methyl, ethyl, n-propyl, n-butyl, n-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl, or n-eicosyl, each of which is unsubstituted. In certain embodiments, R⁴ is methyl, ethyl, n-propyl, n-butyl, n-pentyl, n-hexyl, n-heptyl, n-octyl, n-nonyl, or n-decyl, each of which is unsubstituted. In certain embodiments, R⁴ is n-butyl, n-pentyl, n-hexyl, n-heptyl, n-octyl, n-nonyl, or n-decyl, each of which is unsubstituted.

In certain embodiments, R⁴ is trifluoromethyl, trifluoroethyl, trifluoropropyl, trifluorobutyl, trifluoropentyl, trifluorohexyl, trifluoroheptyl, trifluorooctyl, trifluorononyl, trifluorodecyl, trifluoroundecyl, trifluorododecyl, trifluorotridecyl, trifluorotetradecyl, trifluoro-pentadecyl, trifluorohexadecyl, trifluoroheptadecyl, trifluorooctadecyl, trifluorononadecyl, or trifluoroeicosyl. In certain embodiments, R⁴ is trifluoromethyl, trifluoroethyl, trifluoropropyl, trifluorobutyl, trifluoropentyl, trifluorohexyl, trifluoroheptyl, trifluorooctyl, trifluorononyl, or trifluorodecyl. In certain embodiments, R⁴ is trifluorobutyl, trifluoropentyl, trifluorohexyl, trifluoroheptyl, trifluorooctyl, trifluorononyl, or trifluorodecyl.

In certain embodiments, R⁴ is trifluoromethyl, 2,2,2-trifluoroethyl, 3,3,3-trifluoro-prop-1-yl, 4,4,4-trifluorobut-1-yl, 5,5,5-trifluoropent-1-yl, 6,6,6-trifluorohex-1-yl, 7,7,7-trifluorohept-1-yl, 8,8,8-trifluorooct-1-yl, 9,9,9-trifluoronon-1-yl, 10,10,10-trifluorodec-1-yl, 11,11,11-trifluoroundec-1-yl, 12,12,12-trifluorododec-1-yl, 13,13,13-trifluorotridec-1-yl, 14,14,14-trifluorotetradec-1-yl, 15,15,15-trifluoropentadec-1-yl, 16,16,16-trifluorohexadec-1-yl, 17,17,17-trifluoroheptadec-1-yl, 18,18,18-trifluorooctadec-1-yl, 19,19,19-trifluorononadec-1-yl, or 20,20,20-trifluoroeicosyl. In certain embodiments, R⁴ is trifluoromethyl, 2,2,2-trifluoroethyl, 3,3,3-trifluoroprop-1-yl, 4,4,4-trifluorobut-1-yl, 5,5,5-trifluoropent-1-yl, 6,6,6-trifluorohex-1-yl, 7,7,7-trifluorohept-1-yl, 8,8,8-trifluorooct-1-yl, 9,9,9-trifluoronon-1-yl, or 10,10,10-trifluorodec-1-yl. In certain embodiments, R⁴ is 4,4,4-trifluorobut-1-yl, 5,5,5-trifluoropent-1-yl, 6,6,6-trifluorohex-1-yl, 7,7,7-trifluorohept-1-yl, 8,8,8-trifluorooct-1-yl, 9,9,9-trifluoronon-1-yl, or 10,10,10-trifluorodec-1-yl.

In certain embodiments, R⁴ is C₁₋₂₀ heteroalkyl, optionally substituted with one or more substituents Q. In certain embodiments, R⁴ is 2-(2-(pentyloxy)ethoxy)ethyl or 2-(2-(2-(undecyloxy)ethoxy)ethoxy)ethyl. In certain embodiments, R⁴ is C₂₋₂₀ alkenyl, optionally substituted with one or more substituents Q. In certain embodiments, R⁴ is C₂₋₂₀ alkynyl, optionally substituted with one or more substituents Q.

In certain embodiments, R⁴ is hexyl, octyl, decyl, trifluorohexyl, trifluorooctyl, or trifluorodecyl. In certain embodiments, R⁴ is n-hexyl, n-octyl, n-decyl, 6,6,6-trifluorohex-1-yl, 8,8,8-trifluorooct-1-yl, or 10,10,10-trifluorodec-1-yl. In certain embodiments, R⁴ is n-octyl or 8,8,8-trifluorooct-1-yl. In certain embodiments, R⁴ is n-hexyl, n-octyl, n-decyl, 6,6,6-trifluoro-hex-1-yl, 8,8,8-trifluorooct-1-yl, 10,10,10-trifluorodec-1-yl, 2-(2-(pentyloxy)ethoxy)ethyl, or 2-(2-(2-(undecyloxy)ethoxy)ethoxy)ethyl. In certain embodiments, R⁴ is n-octyl, 8,8,8-trifluoro-oct-1-yl, 2-(2-(pentyloxy)ethoxy)ethyl, or 2-(2-(2-(undecyloxy)ethoxy)ethoxy)ethyl.

In certain embodiments, R⁵ is —C(O)OR^{5a}, wherein R^{5a} is as defined herein. In certain embodiments, R⁵ is —C(O)OH. In certain embodiments, R⁵ is —C(O)O—C₁₋₂₀ alkyl, optionally substituted with one or more substituents Q. In certain embodiments, R⁵ is —C(O)OCH₃. In certain embodiments, R⁵ is —OR^{5a}, wherein R^{5a} is as defined herein. In certain embodiments, R⁵ is hydroxyl. In certain embodiments, R⁵ is C₁₋₆ alkoxy, optionally substituted with one or more substituents Q. In certain embodiments, R⁵ is methoxy. In certain embodiments, R⁵ is —OC(O)R^{5a}, wherein R^{5a} is as defined herein. In certain embodiments, R⁵ is —OC(O)—C₁₋₂₀ alkyl, optionally substituted with one or more substituents Q. In certain embodiments, R⁵ is formoxy or acetoxy.

In certain embodiments, R^{1a} is hydrogen. In certain embodiments, R^{1a} is C₁₋₆ alkyl, optionally substituted with one or more substituents Q. In certain embodiments, R^{1a} is C₁₋₆ heteroalkyl, optionally substituted with one or more substituents Q. In certain embodiments, R^{1a} is C₂₋₆ alkenyl, optionally substituted with one or more substituents Q. In certain embodiments, R^{1a} is C₂₋₆ alkynyl, optionally substituted with one or more substituents Q. In certain embodiments, R^{1a} is C₃₋₁₀ cycloalkyl, optionally substituted with one or more substituents Q. In certain embodiments, R^{1a} is C₆₋₁₄ aryl, optionally substituted with one or more substituents Q. In certain embodiments, R^{1a} is C₇₋₁₅ aralkyl, optionally substituted with one or more substituents Q. In certain embodiments, R^{1a} is heteroaryl, optionally substituted with one or more substituents Q. In certain embodiments, R^{1a} is heterocyclyl, optionally substituted with one or more substituents Q.

In certain embodiments, R^{2a} is hydrogen. In certain embodiments, R^{2a} is C₁₋₆ alkyl, optionally substituted with one or more substituents Q. In certain embodiments, R^{2a} is C₁₋₆ heteroalkyl, optionally substituted with one or more substituents Q. In certain embodiments, R^{2a} is C₂₋₆ alkenyl, optionally substituted with one or more substituents Q. In certain embodiments, R^{2a} is C₂₋₆ alkynyl, optionally substituted with one or more substituents Q. In certain embodiments, R^{2a} is C₃₋₁₀ cycloalkyl, optionally substituted with one or more substituents Q. In certain embodiments, R^{2a} is C₆₋₁₄ aryl, optionally substituted with one or more substituents Q. In certain embodiments, R^{2a} is C₇₋₁₅ aralkyl, optionally substituted with one or more substituents Q. In certain embodiments, R^{2a} is heteroaryl, optionally substituted with one or more substituents Q. In certain embodiments, R^{2a} is heterocyclyl, optionally substituted with one or more substituents Q.

In certain embodiments, L¹ is C₁₋₂₀ alkylene, optionally substituted with one or more substituents Q. In certain embodiments, L¹ is unsubstituted C₁₋₂₀ alkylene. In certain embodiments, L¹ is C₁₋₂₀ alkylene, substituted with one or more substituents Q. In certain embodiments, L¹ is C₁₋₁₀ alkylene, optionally substituted with one or more substituents Q. In certain embodiments, L¹ is unsubstituted C₁₋₁₀ alkylene. In certain embodiments, L¹ is C₁₋₁₀ alkylene, substituted with one or more substituents Q.

In certain embodiments, L¹ is methanediyl, ethanediyl, propanediyl, butanediyl, pentanediyl, hexanediyl, heptanediyl, octanediyl, nonanediyl, decanediyl, undecanediyl, dodecanediyl, tridecanediyl, tetradecanediyl, pentadecanediyl, hexadecanediyl, heptadecanediyl, octadecanediyl, nonadecanediyl, or eicosanediyl, each optionally substituted with one or more substituents Q. In certain embodiments, L¹ is ethanediyl, propanediyl, butanediyl, pentanediyl, hexanediyl, heptanediyl, octanediyl, nonanediyl, or decanediyl, each optionally substituted with one or more substituents Q. In certain embodiments, L¹ is butanediyl, pentanediyl, hexanediyl, heptanediyl, or octanediyl, each optionally substituted with one or more substituents Q.

In certain embodiments, L¹ is methane-1,1-diyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, tetradecane-1,14-diyl, pentadecane-1,15-diyl, hexadecane-1,16-diyl, heptadecane-1,17-diyl, octadecane-1,18-diyl, nonadecane-1,19-diyl, or eicosane-1,20-diyl, each optionally substituted with one or more substituents Q. In certain embodiments, L¹ is ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, or decane-1,10-diyl, each optionally substituted with one or more substituents Q. In certain embodiments, L¹ is butane-1,4-diyl, pentane-1,5-diyl, hexane-1,6-diyl, heptane-1,7-diyl, or octane-1,8-diyl, each optionally substituted with one or more substituents Q.

In certain embodiments, L¹ is methane-1,1-diyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, tetradecane-1,14-diyl, pentadecane-1,15-diyl, hexadecane-1,16-diyl, heptadecane-1,17-diyl, octadecane-1,18-diyl, nonadecane-1,19-diyl, or eicosane-1,20-diyl, each of which is unsubstituted. In certain embodiments, L¹ is ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, or decane-1,10-diyl, each of which is unsubstituted. In certain embodiments, L¹ is butane-1,4-diyl, pentane-1,5-diyl, hexane-1,6-diyl, heptane-1,7-diyl, or octane-1,8-diyl, each of which is unsubstituted.

In certain embodiments, L¹ is C₁₋₂₀ heteroalkylene, optionally substituted with one or more substituents Q. In certain embodiments, L¹ is —(CH₂)₂O(CH₂)₂—, —(CH₂)₂O(CH₂)₃—, —(CH₂)₃O(CH₂)₃—, or —(CH₂)₂O(CH₂)₂O(CH₂)₂—. In certain embodiments, L¹ is —(CH₂)₂O(CH₂)₂—, —(CH₂)₂O(CH₂)₃—, or —(CH₂)₃O(CH₂)₃—. In certain embodiments, L¹ is —(CH₂)₂O(CH₂)₂—. In certain embodiments, L¹ is —(CH₂)₂O(CH₂)₃—. In certain embodiments, L¹ is —(CH₂)₃O(CH₂)₃—. In certain embodiments, L¹ is —(CH₂)₂O(CH₂)₂O(CH₂)₂—. In certain embodiments, L¹ is —N(H)—C₁₋₂₀ alkenylene or —N(H)—C₁₋₂₀ heteroalkenylene, each optionally substituted with one or more substituents Q. In certain embodiments, L¹ is —N(H)—C₁₋₂₀ alkenylene, optionally substituted with one or more substituents Q. In certain embodiments, L¹ is —N(H)—C₁₋₁₀ alkenylene, optionally substituted with one or more substituents Q. In certain embodiments, L¹ is —N(H)(CH₂)₂—, —N(H)(CH₂)₃—, —N(H)(CH₂)₄—, —N(H)(CH₂)₅—, —N(H)(CH₂)₆—, —N(H)(CH₂)₇—, or —N(H)(CH₂)₈—, each optionally substituted with one or more substituents Q. In certain embodiments, L¹ is —N(H)—C₁₋₂₀ heteroalkenylene, optionally substituted with one or more substituents Q. In certain embodiments, L¹ is C₂₋₂₀ alkenylene, optionally substituted with one or more substituents Q. In certain embodiments, L¹ is C₂₋₂₀ alkynylene, optionally substituted with one or more substituents Q.

In certain embodiments, L¹ is pentanediyl, hexanediyl, heptanediyl, —(CH₂)₂O(CH₂)₂—, —(CH₂)₂O(CH₂)₃—, or —(CH₂)₂O(CH₂)₂O(CH₂)₂—. In certain embodiments, L¹ is pentanediyl, hexanediyl, heptanediyl, or —(CH₂)₂O(CH₂)₃—. In certain embodiments, L¹ is pentane-1,5-diyl, hexane-1,6-diyl, heptane-1,7-diyl, —(CH₂)₂O(CH₂)₂—, —(CH₂)₂O(CH₂)₃—, or —(CH₂)₂O(CH₂)₂O(CH₂)₂—. In certain embodiments, L¹ is pentane-1,5-diyl, hexane-1,6-diyl, heptane-1,7-diyl, or —(CH₂)₂O(CH₂)₃—. In certain embodiments, L¹ is pentane-1,5-diyl, hexane-1,6-diyl, heptane-1,7-diyl, —(CH₂)₂O(CH₂)₂—, —(CH₂)₂O(CH₂)₃—, —(CH₂)₂O(CH₂)₂O(CH₂)₂—, or —N(H)(CH₂)₆—.

In certain embodiments, L² is C₁₋₂₀ alkylene, optionally substituted with one or more substituents Q. In certain embodiments, L² is unsubstituted C₁₋₂₀ alkylene. In certain embodiments, L² is C₁₋₂₀ alkylene, substituted with one or more substituents Q.

In certain embodiments, L² is methanediyl, ethanediyl, propanediyl, butanediyl, pentanediyl, hexanediyl, heptanediyl, octanediyl, nonanediyl, decanediyl, undecanediyl, dodecanediyl, tridecanediyl, tetradecanediyl, pentadecanediyl, hexadecanediyl, heptadecanediyl, octadecanediyl, nonadecanediyl, or eicosanediyl, each optionally substituted with one or more substituents Q. In certain embodiments, L² is ethanediyl, propanediyl, butanediyl, pentanediyl, hexanediyl, heptanediyl, octanediyl, nonanediyl, or decanediyl, each optionally substituted with one or more substituents Q. In certain embodiments, L² is butanediyl, pentanediyl, hexanediyl, heptanediyl, or octanediyl, each optionally substituted with one or more substituents Q.

In certain embodiments, L² is methane-1,1-diyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, tetradecane-1,14-diyl, pentadecane-1,15-diyl, hexadecane-1,16-diyl, heptadecane-1,17-diyl, octadecane-1,18-diyl, nonadecane-1,19-diyl, or eicosane-1,20-diyl, each optionally substituted with one or more substituents Q. In certain embodiments, L² is ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, or decane-1,10-diyl, each optionally substituted with one or more substituents Q. In certain embodiments, L² is butane-1,4-diyl, pentane-1,5-diyl, hexane-1,6-diyl, heptane-1,7-diyl, or octane-1,8-diyl, each optionally substituted with one or more substituents Q.

In certain embodiments, L² is methane-1,1-diyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, tetradecane-1,14-diyl, pentadecane-1,15-diyl, hexadecane-1,16-diyl, heptadecane-1,17-diyl, octadecane-1,18-diyl, nonadecane-1,19-diyl, or eicosane-1,20-diyl, each of which is unsubstituted. In certain embodiments, L² is ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, or decane-1,10-diyl, each of which is unsubstituted. In certain embodiments, L² is butane-1,4-diyl, pentane-1,5-diyl, hexane-1,6-diyl, heptane-1,7-diyl, or octane-1,8-diyl, each of which is unsubstituted.

In certain embodiments, L² is C₁₋₂₀ heteroalkylene, optionally substituted with one or more substituents Q. In certain embodiments, L² is —(CH₂)₂O(CH₂)₂—, —(CH₂)₂O(CH₂)₃—, —(CH₂)₃O(CH₂)₃—, or —(CH₂)₂O(CH₂)₂O(CH₂)₂—. In certain embodiments, L² is —(CH₂)₂O(CH₂)₂—, —(CH₂)₂O(CH₂)₃—, or —(CH₂)₃O(CH₂)₃—. In certain embodiments, L² is —(CH₂)₂O(CH₂)₂—. In certain embodiments, L² is —(CH₂)₂O(CH₂)₃—. In certain embodiments, L² is —(CH₂)₃O(CH₂)₃—. In certain embodiments, L² is —(CH₂)₂O(CH₂)₂O(CH₂)₂—. In certain embodiments, L² is —N(H)—C₁₋₂₀ alkenylene or —N(H)—C₁₋₂₀ heteroalkenylene, each optionally substituted with one or more substituents Q. In certain embodiments, L² is —N(H)—C₁₋₂₀ alkenylene, optionally substituted with one or more substituents Q. In certain embodiments, L² is —N(H)—C₁₋₁₀ alkenylene, optionally substituted with one or more substituents Q. In certain embodiments, L² is —N(H)(CH₂)₂—, —N(H)(CH₂)₃—, —N(H)(CH₂)₄—, —N(H)(CH₂)₅—, —N(H)(CH₂)₆—, —N(H)(CH₂)₇—, or —N(H)(CH₂)₈—, each optionally substituted with one or more substituents Q. In certain embodiments, L² is —N(H)—C₁₋₂₀ heteroalkenylene, optionally substituted with one or more substituents Q. In certain embodiments, L² is C₂₋₂₀ alkenylene, optionally substituted with one or more substituents Q. In certain embodiments, L² is C₂₋₂₀ alkynylene, optionally substituted with one or more substituents Q.

In certain embodiments, L² is pentanediyl, hexanediyl, heptanediyl, —(CH₂)₂O(CH₂)₂—, —(CH₂)₂O(CH₂)₃—, or —(CH₂)₂O(CH₂)₂O(CH₂)₂—. In certain embodiments, L² is pentanediyl, hexanediyl, heptanediyl, or —(CH₂)₂O(CH₂)₃—. In certain embodiments, L² is pentane-1,5-diyl, hexane-1,6-diyl, heptane-1,7-diyl, —(CH₂)₂O(CH₂)₂—, —(CH₂)₂O(CH₂)₃—, or —(CH₂)₂O(CH₂)₂O(CH₂)₂—. In certain embodiments, L² is pentane-1,5-diyl, hexane-1,6-diyl, heptane-1,7-diyl, or —(CH₂)₂O(CH₂)₃—. In certain embodiments, L² is pentane-1,5-diyl, hexane-1,6-diyl, heptane-1,7-diyl, —(CH₂)₂O(CH₂)₂—, —(CH₂)₂O(CH₂)₃—, —(CH₂)₂O(CH₂)₂O(CH₂)₂—, or —N(H)(CH₂)₆—.

In certain embodiments, L³ is C₁₋₂₀ alkylene, optionally substituted with one or more substituents Q. In certain embodiments, L³ is unsubstituted C₁₋₂₀ alkylene. In certain embodiments, L³ is C₁₋₂₀ alkylene, substituted with one or more substituents Q.

In certain embodiments, L³ is methanediyl, ethanediyl, propanediyl, butanediyl, pentanediyl, hexanediyl, heptanediyl, octanediyl, nonanediyl, decanediyl, undecanediyl, dodecanediyl, tridecanediyl, tetradecanediyl, pentadecanediyl, hexadecanediyl, heptadecanediyl, octadecanediyl, nonadecanediyl, or eicosanediyl, each optionally substituted with one or more substituents Q. In certain embodiments, L³ is ethanediyl, propanediyl, butanediyl, pentanediyl, hexanediyl, heptanediyl, octanediyl, nonanediyl, or decanediyl, each optionally substituted with one or more substituents Q. In certain embodiments, L³ is ethanediyl, propanediyl, butanediyl, pentanediyl, or hexanediyl, each optionally substituted with one or more substituents Q. In certain embodiments, L³ is ethanediyl, propanediyl, butanediyl, or pentanediyl, each optionally substituted with one or more substituents Q.

In certain embodiments, L³ is methane-1,1-diyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, tetradecane-1,14-diyl, pentadecane-1,15-diyl, hexadecane-1,16-diyl, heptadecane-1,17-diyl, octadecane-1,18-diyl, nonadecane-1,19-diyl, or eicosane-1,20-diyl, each optionally substituted with one or more substituents Q. In certain embodiments, L³ is ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, or decane-1,10-diyl, each optionally substituted with one or more substituents Q. In certain embodiments, L³ is ethane-1,2-diyl, propane-1,3-diyl, butane-1,4-diyl, pentane-1,5-diyl, or hexane-1,6-diyl, each optionally substituted with one or more substituents Q. In certain embodiments, L³ is ethane-1,2-diyl, propane-1,3-diyl, butane-1,4-diyl, or pentane-1,5-diyl, each optionally substituted with one or more substituents Q.

In certain embodiments, L³ is methane-1,1-diyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, tetradecane-1,14-diyl, pentadecane-1,15-diyl, hexadecane-1,16-diyl, heptadecane-1,17-diyl, octadecane-1,18-diyl, nonadecane-1,19-diyl, or eicosane-1,20-diyl, each of which is unsubstituted. In certain embodiments, L³ is ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, or decane-1,10-diyl, each of which is unsubstituted. In certain embodiments, L³ is ethane-1,2-diyl, propane-1,3-diyl, butane-1,4-diyl, pentane-1,5-diyl, or hexane-1,6-diyl, each of which is unsubstituted. In certain embodiments, L³ is ethane-1,2-diyl, propane-1,3-diyl, butane-1,4-diyl, or pentane-1,5-diyl, each of which is unsubstituted.

In certain embodiments, L³ is C₁₋₂₀ heteroalkylene, optionally substituted with one or more substituents Q. In certain embodiments, L³ is —(CH₂)₂O(CH₂)₂—, —(CH₂)₂O(CH₂)₃—, or —(CH₂)₃O(CH₂)₃—. In certain embodiments, L³ is —(CH₂)₂O(CH₂)₂—. In certain embodiments, L³ is —(CH₂)₂O(CH₂)₃—. In certain embodiments, L³ is —(CH₂)₃O(CH₂)₃—. In certain embodiments, L³ is C₂₋₂₀ alkenylene, optionally substituted with one or more substituents Q. In certain embodiments, L³ is C₂₋₂₀ alkynylene, optionally substituted with one or more substituents Q.

In certain embodiments, L³ is ethanediyl or butanediyl. In certain embodiments, L³ is ethane-1,2-diyl or butane-1,4-diyl.

In certain embodiments, L^{1a} is C₁₋₂₀ alkylene, optionally substituted with one or more substituents Q. In certain embodiments, L^{1a} is unsubstituted C₁₋₂₀ alkylene. In certain embodiments, L^{1a} is C₁₋₂₀ alkylene, substituted with one or more substituents Q. In certain embodiments, L^{1a} is C₁₋₁₀ alkylene, optionally substituted with one or more substituents Q. In certain embodiments, L^{1a} is unsubstituted C₁₋₁₀ alkylene. In certain embodiments, L^{1a} is C₁₋₁₀ alkylene, substituted with one or more substituents Q.

In certain embodiments, L^{1a} is methanediyl, ethanediyl, propanediyl, butanediyl, pentanediyl, hexanediyl, heptanediyl, octanediyl, nonanediyl, decanediyl, undecanediyl, dodecanediyl, tridecanediyl, tetradecanediyl, pentadecanediyl, hexadecanediyl, heptadecanediyl, octadecanediyl, nonadecanediyl, or eicosanediyl, each optionally substituted with one or more substituents Q. In certain embodiments, L^{1a} is ethanediyl, propanediyl, butanediyl, pentanediyl, hexanediyl, heptanediyl, octanediyl, nonanediyl, or decanediyl, each optionally substituted with one or more substituents Q. In certain embodiments, L^{1a} is butanediyl, pentanediyl, hexanediyl, heptanediyl, or octanediyl, each optionally substituted with one or more substituents Q.

In certain embodiments, L^{1a} is methane-1,1-diyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, tetradecane-1,14-diyl, pentadecane-1,15-diyl, hexadecane-1,16-diyl, heptadecane-1,17-diyl, octadecane-1,18-diyl, nonadecane-1,19-diyl, or eicosane-1,20-diyl, each optionally substituted with one or more substituents Q. In certain embodiments, L^{1a} is ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, or decane-1,10-diyl, each optionally substituted with one or more substituents Q. In certain embodiments, L^{1a} is butane-1,4-diyl, pentane-1,5-diyl, hexane-1,6-diyl, heptane-1,7-diyl, or octane-1,8-diyl, each optionally substituted with one or more substituents Q.

In certain embodiments, L^{1a} is methane-1,1-diyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, tetradecane-1,14-diyl, pentadecane-1,15-diyl, hexadecane-1,16-diyl, heptadecane-1,17-diyl, octadecane-1,18-diyl, nonadecane-1,19-diyl, or eicosane-1,20-diyl, each of which is unsubstituted. In certain embodiments, L^{1a} is ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, or decane-1,10-diyl, each of which is unsubstituted. In certain embodiments, L^{1a} is butane-1,4-diyl, pentane-1,5-diyl, hexane-1,6-diyl, heptane-1,7-diyl, or octane-1,8-diyl, each of which is unsubstituted.

In certain embodiments, L^{1a} is C₁₋₂₀ heteroalkylene, optionally substituted with one or more substituents Q. In certain embodiments, L^{1a} is —(CH₂)₂O(CH₂)₂—, —(CH₂)₂O(CH₂)₃—, —(CH₂)₃O(CH₂)₃—, or —(CH₂)₂O(CH₂)₂O(CH₂)₂—. In certain embodiments, L^{1a} is —(CH₂)₂O(CH₂)₂—, —(CH₂)₂O(CH₂)₃—, or —(CH₂)₃O(CH₂)₃—. In certain embodiments, L^{1a} is —(CH₂)₂O(CH₂)₂—. In certain embodiments, L^{1a} is —(CH₂)₂O(CH₂)₃—. In certain embodiments, L^{1a} is —(CH₂)₃O(CH₂)₃—. In certain embodiments, L^{1a} is —(CH₂)₂O(CH₂)₂O(CH₂)₂—.

In certain embodiments, L^{1a} is pentanediyl, hexanediyl, heptanediyl, —(CH₂)₂O(CH₂)₂—, —(CH₂)₂O(CH₂)₃—, or —(CH₂)₂O(CH₂)₂O(CH₂)₂—. In certain embodiments, L^{1a} is pentane-1,5-diyl, hexane-1,6-diyl, heptane-1,7-diyl, —(CH₂)₂O(CH₂)₂—, —(CH₂)₂O(CH₂)₃—, or —(CH₂)₂O(CH₂)₂O(CH₂)₂—.

In certain embodiments, L^{2a} is C₁₋₂₀ alkylene, optionally substituted with one or more substituents Q. In certain embodiments, L^{2a} is unsubstituted C₁₋₂₀ alkylene. In certain embodiments, L^{2a} is C₁₋₂₀ alkylene, substituted with one or more substituents Q. In certain embodiments, L^{2a} is C₁₋₁₀ alkylene, optionally substituted with one or more substituents Q. In certain embodiments, L^{2a} is unsubstituted C₁₋₁₀ alkylene. In certain embodiments, L^{2a} is C₁₋₁₀ alkylene, substituted with one or more substituents Q.

In certain embodiments, L^{2a} is methanediyl, ethanediyl, propanediyl, butanediyl, pentanediyl, hexanediyl, heptanediyl, octanediyl, nonanediyl, decanediyl, undecanediyl, dodecanediyl, tridecanediyl, tetradecanediyl, pentadecanediyl, hexadecanediyl, heptadecanediyl, octadecanediyl, nonadecanediyl, or eicosanediyl, each optionally substituted with one or more substituents Q. In certain embodiments, L^{2a} is ethanediyl, propanediyl, butanediyl, pentanediyl, hexanediyl, heptanediyl, octanediyl, nonanediyl, or decanediyl, each optionally substituted with one or more substituents Q. In certain embodiments, L^{2a} is butanediyl, pentanediyl, hexanediyl, heptanediyl, or octanediyl, each optionally substituted with one or more substituents Q.

In certain embodiments, L^{2a} is methane-1,1-diyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, tetradecane-1,14-diyl, pentadecane-1,15-diyl, hexadecane-1,16-diyl, heptadecane-1,17-diyl, octadecane-1,18-diyl, nonadecane-1,19-diyl, or eicosane-1,20-diyl, each optionally substituted with one or more substituents Q. In certain embodiments, L^{2a} is ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, or decane-1,10-diyl, each optionally substituted with one or more substituents Q. In certain embodiments, L^{2a} is butane-1,4-diyl, pentane-1,5-diyl, hexane-1,6-diyl, heptane-1,7-diyl, or octane-1,8-diyl, each optionally substituted with one or more substituents Q.

In certain embodiments, L^{2a} is methane-1,1-diyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, tetradecane-1,14-diyl, pentadecane-1,15-diyl, hexadecane-1,16-diyl, heptadecane-1,17-diyl, octadecane-1,18-diyl, nonadecane-1,19-diyl, or eicosane-1,20-diyl, each of which is unsubstituted. In certain embodiments, L^{2a} is ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, or decane-1,10-diyl, each of which is unsubstituted. In certain embodiments, L^{2a} is butane-1,4-diyl, pentane-1,5-diyl, hexane-1,6-diyl, heptane-1,7-diyl, or octane-1,8-diyl, each of which is unsubstituted.

In certain embodiments, L^{2a} is C₁₋₂₀ heteroalkylene, optionally substituted with one or more substituents Q. In certain embodiments, L^{2a} is —(CH₂)₂O(CH₂)₂—, —(CH₂)₂O(CH₂)₃—, —(CH₂)₃O(CH₂)₃—, or —(CH₂)₂O(CH₂)₂O(CH₂)₂—. In certain embodiments, L^{2a} is —(CH₂)₂O(CH₂)₂—, —(CH₂)₂O(CH₂)₃—, or —(CH₂)₃O(CH₂)₃—. In certain embodiments, L^{2a} is —(CH₂)₂O(CH₂)₂—. In certain embodiments, L^{2a} is —(CH₂)₂O(CH₂)₃—. In certain embodiments, L^{2a} is —(CH₂)₃O(CH₂)₃—. In certain embodiments, L^{2a} is —(CH₂)₂O(CH₂)₂O(CH₂)₂—.

In certain embodiments, L^{2a} is pentanediyl, hexanediyl, heptanediyl, —(CH₂)₂O(CH₂)₂—, —(CH₂)₂O(CH₂)₃—, or —(CH₂)₂O(CH₂)₂O(CH₂)₂—. In certain embodiments, L^{2a} is pentane-1,5-diyl, hexane-1,6-diyl, heptane-1,7-diyl, —(CH₂)₂O(CH₂)₂—, —(CH₂)₂O(CH₂)₃—, or —(CH₂)₂O(CH₂)₂O(CH₂)₂—.

In certain embodiments, U is —C(O)—. In certain embodiments, U is —C(O)O—. In certain embodiments, U is —OC(O)O—.

In certain embodiments, V is —C(O)—. In certain embodiments, V is —C(O)O—. In certain embodiments, V is —OC(O)O—.

In certain embodiments, X is C(H). In certain embodiments, X is N.

In certain embodiments, Y is C(H). In certain embodiments, Y is N.

In one embodiment, provided herein is a compound of:
8,8′-((2-hydroxyethyl)azanediyl)bis(N-hexyl-N-octyloctanamide) A1;
((2-hydroxyethyl)azanediyl)bis(hexane-6,1-diyl) bis(hexyl(octyl)carbamate) A2;
N-(2-hydroxyethyl)-N,N-di(7-(pentadecan-7-oxycarbonyloxy)heptyl)amine A3;
((((2-hydroxyethyl)azanediyl)bis(ethane-2,1-diyl))bis(oxy))bis(ethane-2,1-diyl) bis(2-hexyldecanoate) A4;
di(heptadecan-9-yl) 10-(2-hydroxyethyl)-4,7,13,16-tetraoxa-10-azanonadecanedioate A5;
bis(1,1,1,15,15,15-hexafluoropentadecan-7-yl) 7,7′-((2-hydroxyethyl)-azanediyl)diheptanoate A6;
1,1,1,15,15,15-hexafluoropentadecan-7-yl 8-((2-hydroxyethyl)(6-oxo-6-((11,11,11-trifluoroundecyl)oxy)hexyl)amino)octanoate A7;
pentadecan-7-yl 8-((2-hydroxyethyl)(6-oxo-6-((11,11,11-trifluoroundecyl)oxy)-hexyl)amino)octanoate A8;
1,1,1,17,17,17-hexafluoroheptadecan-9-yl 8-((2-hydroxyethyl)(6-oxo-6-((11,11,11-trifluoroundecyl)oxy)hexyl)amino)octanoate A9;
heptadecan-9-yl 8-((2-hydroxyethyl)(6-oxo-6-((11,11,11-trifluoroundecyl)oxy)-hexyl)amino)octanoate A10;
8,8′-((3-hydroxypropyl)azanediyl)bis(N-hexyl-N-octyloctanamide) A11;
((3-hydroxypropyl)azanediyl)bis(hexane-6,1-diyl) bis(hexyl(octyl)carbamate) A12;
N-(3-hydroxypropyl)-N,N-di(7-(pentadecan-7-oxycarbonyloxy)heptyl)amine A13;
((((3-hydroxypropyl)azanediyl)bis(propane-3,1-diyl))bis(oxy))bis(ethane-2,1-diyl) bis(2-hexyldecanoate) A14;
di(pentadecan-7-yl) 3,3′-((((3-hydroxypropyl)azanediyl)bis(propane-3,1-diyl))bis(oxy))dipropionate A15;
bis(1,1,1,15,15,15-hexafluoropentadecan-7-yl) 7,7′-((3-hydroxypropyl)-azanediyl)diheptanoate A16;
1,1,1,15,15,15-hexafluoropentadecan-7-yl 8-((3-hydroxypropyl)(6-oxo-6-((11,11,11-trifluoroundecyl)oxy)hexyl)amino)octanoate A17;
pentadecan-7-yl 8-((3-hydroxypropyl)(6-oxo-6-((11,11,11-trifluoroundecyl)oxy)-hexyl)amino)octanoate A18;
1,1,1,17,17,17-hexafluoroheptadecan-9-yl 8-((3-hydroxypropyl)(6-oxo-6-((11,11,11-trifluoroundecyl)oxy)hexyl)amino)octanoate A19;
heptadecan-9-yl 8-((3-hydroxypropyl)(6-oxo-6-((11,11,11-trifluoroundecyl)oxy)-hexyl)amino)octanoate A20;
8,8′-((4-hydroxybutyl)azanediyl)bis(N-hexyl-N-octyloctanamide) A21;
((4-hydroxybutyl)azanediyl)bis(hexane-6,1-diyl) bis(hexyl(octyl)carbamate) A22;
N-(4-hydroxybutyl)-N,N-di(7-(pentadecan-7-oxycarbonyloxy)heptyl)amine A23;
((((4-hydroxybutyl)azanediyl)bis(propane-3,1-diyl))bis(oxy))bis(ethane-2,1-diyl) bis(2-hexyldecanoate) A24;
di(pentadecan-7-yl) 3,3′-((((4-hydroxybutyl)azanediyl)bis(propane-3,1-diyl))bis(oxy))dipropionate A25;
bis(1,1,1,15,15,15-hexafluoropentadecan-7-yl) 7,7′-((4-hydroxybutyl)-azanediyl)diheptanoate A26;
1,1,1,15,15,15-hexafluoropentadecan-7-yl 8-((4-hydroxybutyl)(6-oxo-6-((11,11,11-trifluoroundecyl)oxy)hexyl)amino)octanoate A27;
pentadecan-7-yl 8-((4-hydroxybutyl)(6-oxo-6-((11,11,11-trifluoroundecyl)oxy)-hexyl)amino)octanoate A28;
1,1,1,17,17,17-hexafluoroheptadecan-9-yl 8-((4-hydroxybutyl)(6-oxo-6-((11,11,11-trifluoroundecyl)oxy)hexyl)amino)octanoate A29;
heptadecan-9-yl 8-((4-hydroxybutyl)(6-oxo-6-((11,11,11-trifluoroundecyl)oxy)-hexyl)amino)octanoate A30;
8,8′-((5-hydroxypentyl)azanediyl)bis(N-hexyl-N-octyloctanamide) A31;
((5-hydroxypentyl)azanediyl)bis(hexane-6,1-diyl) bis(hexyl(octyl)carbamate) A32;
N-(5-hydroxypentyl)-N,N-di(7-(pentadecan-7-oxycarbonyloxy)heptyl)amine A33;
((((5-hydroxypentyl)azanediyl)bis(propane-3,1-diyl))bis(oxy))bis(ethane-2,1-diyl) bis(2-hexyldecanoate) A34;
di(pentadecan-7-yl) 3,3′-((((5-hydroxypentyl)azanediyl)bis(propane-3,1-diyl))bis(oxy))dipropionate A35;
bis(1,1,1,15,15,15-hexafluoropentadecan-7-yl) 7,7′-((5-hydroxypentyl)-azanediyl)diheptanoate A36;
1,1,1,15,15,15-hexafluoropentadecan-7-yl 8-((5-hydroxypentyl)(6-oxo-6-((11,11,11-trifluoroundecyl)oxy)hexyl)amino)octanoate A37;
pentadecan-7-yl 8-((5-hydroxypentyl)(6-oxo-6-((11,11,11-trifluoroundecyl)oxy)-hexyl)amino)octanoate A38;
1,1,1,17,17,17-hexafluoroheptadecan-9-yl 8-((5-hydroxypentyl)(6-oxo-6-((11,11,11-trifluoroundecyl)oxy)hexyl)amino)octanoate A39;
heptadecan-9-yl 8-((5-hydroxypentyl)(6-oxo-6-((11,11,11-trifluoroundecyl)oxy)-hexyl)amino)octanoate A40;
N-(2-hydroxyethyl)-N,N-di(5-(pentadecan-7-oxycarbonyloxy)pentyl)amine A41;
N-(2-hydroxyethyl)-N,N-di(7-(heptadecan-9-oxycarbonyloxy)heptyl)amine A42;
((((2-hydroxyethyl)azanediyl)bis(propane-3,1-diyl))bis(oxy))bis(ethane-2,1-diyl) bis(2-hexyldecanoate) A43;
di(pentadecan-7-yl) 3,3′-((((2-hydroxyethyl)azanediyl)bis(propane-3,1-diyl))bis(oxy))dipropionate A44;
di(pentadecan-7-yl) 10-(2-hydroxyethyl)-4,7,13,16-tetraoxa-10-azanonadecane-dioate A45;
heptadecan-9-yl (6-((3-hydroxypropyl)(6-(((undecyloxy)carbonyl)amino)hexyl)-amino)hexyl)carbamate A46;
heptadecan-9-yl (6-((4-hydroxybutyl)(6-(((undecyloxy)carbonyl)amino)hexyl)-amino)hexyl)carbamate A47;
N,N-(((4-hydroxybutyl)azanediyl)bis(hexane-6,1-diyl))bis(2-hexyldecanamide) A48;
N,N-(((2-hydroxyethyl)azanediyl)bis(hexane-6,1-diyl))bis(2-hexyldecanamide) A49;
((((2-hydroxyethyl)azanediyl)bis(ethane-2,1-diyl))bis(oxy))bis(ethane-2,1-diyl) bis(2-hexyldecanoate) A50;
di(heptadecan-9-yl) 10-(2-hydroxyethyl)-4,7,13,16-tetraoxa-10-azanonadecane-dioate A51;
8,8′-((2-hydroxyethyl)azanediyl)bis(N-hexyl-N-octyloctanamide) A52;
heptadecan-9-yl 8-((2-hydroxyethyl)(3-oxo-4,7,10,13-tetraoxatetracosyl)amino)-octanoate A53; or
heptadecan-9-yl 8-((2-hydroxyethyl)(6-oxo-6-(2-(2-(pentyloxy)ethoxy)ethoxy)-hexyl)amino)octanoate A54;
   or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof.

In another embodiment, provided herein is a compound of:
heptadecan-9-yl 6-((2-hydroxyethyl)(5-(nonyloxy)-5-oxopentyl)amino)hexanoate B1;
heptadecan-9-yl 6-((6-(heptyloxy)-6-oxohexyl)(2-hydroxyethyl)amino)hexanoate B2;
heptadecan-9-yl 6-((2-hydroxyethyl)(6-oxo-6-(undecyloxy)hexyl)amino)-hexanoate B3;
heptadecan-9-yl 7-((7-(heptyloxy)-7-oxoheptyl)(2-hydroxyethyl)amino)-heptanoate B4;
heptadecan-9-yl 7-((2-hydroxyethyl)(7-(nonyloxy)-7-oxoheptyl)amino)-heptanoate B5;
heptadecan-9-yl 7-((2-hydroxyethyl)(7-oxo-7-(undecyloxy)heptyl)amino)-heptanoate B6;
heptadecan-9-yl 8-((8-(heptyloxy)-8-oxooctyl)(2-hydroxyethyl)amino)octanoate B7;
heptadecan-9-yl 8-((2-hydroxyethyl)(8-oxo-8-(undecyloxy)octyl)amino)-octanoate B8;
heptadecan-9-yl 7-((2-hydroxyethyl)(6-oxo-6-(undecyloxy)hexyl)amino)-heptanoate B9;
heptadecan-9-yl 8-((6-(heptyloxy)-6-oxohexyl)(2-hydroxyethyl)amino)octanoate B10;
heptadecan-9-yl 8-((2-hydroxyethyl)(6-(nonyloxy)-6-oxohexyl)amino)octanoate B11;
heptyl 7-((6-(heptadecan-9-yloxy)-6-oxohexyl)(2-hydroxyethyl)amino)-heptanoate B12;
undecyl 7-((6-(heptadecan-9-yloxy)-6-oxohexyl)(2-hydroxyethyl)amino)-heptanoate B13;
heptyl 8-((6-(heptadecan-9-yloxy)-6-oxohexyl)(2-hydroxyethyl)amino)octanoate B14;
nonyl 8-((6-(heptadecan-9-yloxy)-6-oxohexyl)(2-hydroxyethyl)amino)octanoate B15;
undecyl 8-((6-(heptadecan-9-yloxy)-6-oxohexyl)(2-hydroxyethyl)amino)-octanoate B16;
nonyl 8-((7-(heptadecan-9-yloxy)-7-oxoheptyl)(2-hydroxyethyl)amino)octanoate B17;
heptadecan-9-yl 10-((2-hydroxyethyl)(8-(nonyloxy)-8-oxooctyl)amino)decanoate B18;
heptadecan-9-yl 10-((2-hydroxyethyl)(10-oxo-10-(undecyloxy)decyl)amino)-decanoate B19;
heptadecan-9-yl 6-((6-(heptyloxy)-6-oxohexyl)(3-hydroxypropyl)amino)-hexanoate B20;
heptadecan-9-yl 6-((3-hydroxypropyl)(6-(nonyloxy)-6-oxohexyl)amino)-hexanoate B21;
heptadecan-9-yl 7-((7-(heptyloxy)-7-oxoheptyl)(3-hydroxypropyl)amino)-heptanoate B22;
heptadecan-9-yl 8-((3-hydroxypropyl)(8-(nonyloxy)-8-oxooctyl)amino)octanoate B23;
heptadecan-9-yl 8-((3-hydroxypropyl)(8-oxo-8-(undecyloxy)octyl)amino)-octanoate B24;
heptadecan-9-yl 7-((3-hydroxypropyl)(6-(nonyloxy)-6-oxohexyl)amino)-heptanoate B25;
heptadecan-9-yl 8-((6-(heptyloxy)-6-oxohexyl)(3-hydroxypropyl)amino)-octanoate B26;
heptadecan-9-yl 8-((3-hydroxypropyl)(6-oxo-6-(undecyloxy)hexyl)amino)-octanoate B27;
di(heptadecan-9-yl) 6,6′-((2-hydroxyethyl)azanediyl)dihexanoate B28;
di(heptadecan-9-yl) 7,7′-((2-hydroxyethyl)azanediyl)diheptanoate B29;
((2-hydroxyethyl)azanediyl)bis(hexane-6,1-diyl) bis(2-hexyldecanoate) B30;
7-((2-hydroxyethyl)(8-oxo-8-(pentadecan-7-yloxy)octyl)amino)heptyl 2-hexyldecanoate B31;
((2-hydroxyethyl)azanediyl)bis(octane-8,1-diyl) bis(2-hexyldecanoate) B32;
((3-hydroxypropyl)azanediyl)bis(hexane-6,1-diyl) bis(2-hexyldecanoate) B33;
((3-hydroxypropyl)azanediyl)bis(heptane-7,1-diyl) bis(2-hexyldecanoate) B34;
((3-hydroxypropyl)azanediyl)bis(octane-8,1-diyl) bis(2-hexyldecanoate) B35;
((4-hydroxybutyl)azanediyl)bis(heptane-7,1-diyl) bis(2-hexyldecanoate) B36;
((4-hydroxybutyl)azanediyl)bis(octane-8,1-diyl) bis(2-hexyldecanoate) B37;
((5-hydroxypentyl)azanediyl)bis(hexane-6,1-diyl) bis(2-hexyldecanoate) B38;
((5-hydroxypentyl)azanediyl)bis(heptane-7,1-diyl) bis(2-hexyldecanoate) B39;
((5-hydroxypentyl)azanediyl)bis(octane-8,1-diyl) bis(2-hexyldecanoate) B40;
di(heptadecan-9-yl) 8,8′-((2-hydroxyethyl)azanediyl)dioctanoate B41;
di(heptadecan-9-yl) 8,8′-((4-hydroxybutyl)azanediyl)dioctanoate B42;
di(heptadecan-9-yl) 8,8′-((3-(dimethylamino)propyl)azanediyl)dioctanoate B43;
heptadecan-9-yl (Z)-8-((2-hydroxyethyl)(6-(non-2-en-1-yloxy)-6-oxohexyl)-amino)octanoate B44;
di(heptadecan-9-yl) 8,8′-((2-hydroxyethyl)azanediyl)dioctanoate B45;
((2-(2-methoxyethoxy)ethyl)azanediyl)bis(hexane-6,1-diyl) bis(2-hexyl decanoate) B46;
di(pentadecan-7-yl) 8,8′-((2-hydroxyethyl)azanediyl)dioctanoate B47; or
5-((2-hydroxyethyl)(7-((2-octyldecanoyl)oxy)heptyl)amino)pentyl dodecanoate B48;
   or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, or hydrate thereof.

In certain embodiments, a compound provided herein is deuterium-enriched. In certain embodiments, a compound provided herein is carbon-13 enriched. In certain embodiments, a compound provided herein is carbon-14 enriched. In certain embodiments, a compound provided herein contains one or more less prevalent isotopes for other elements, including, but not limited to, ¹⁵N for nitrogen; ¹⁷O or ¹⁸O for oxygen, and ³⁴S, ³⁵S, or ³⁶S for sulfur.

In certain embodiments, a compound provided herein has an isotopic enrichment factor of no less than about 5, no less than about 10, no less than about 20, no less than about 50, no less than about 100, no less than about 200, no less than about 500, no less than about 1,000, no less than about 2,000, no less than about 5,000, or no less than about 10,000. In any events, however, an isotopic enrichment factor for a specified isotope is no greater than the maximum isotopic enrichment factor for the specified isotope, which is the isotopic enrichment factor when a compound at a given position is 100% enriched with the specified isotope. Thus, the maximum isotopic enrichment factor is different for different isotopes. The maximum isotopic enrichment factor is 6,410 for deuterium and 90 for carbon-13.

In certain embodiments, a compound provided herein has a deuterium enrichment factor of no less than about 64 (about 1% deuterium enrichment), no less than about 130 (about 2% deuterium enrichment), no less than about 320 (about 5% deuterium enrichment), no less than about 640 (about 10% deuterium enrichment), no less than about 1,300 (about 20% deuterium enrichment), no less than about 3,200 (about 50% deuterium enrichment), no less than about 4,800 (about 75% deuterium enrichment), no less than about 5,130 (about 80% deuterium enrichment), no less than about 5,450 (about 85% deuterium enrichment), no less than about 5,770 (about 90% deuterium enrichment), no less than about 6,090 (about 95% deuterium enrichment), no less than about 6,220 (about 97% deuterium enrichment), no less than about 6,280 (about 98% deuterium enrichment), no less than about 6,350 (about 99% deuterium enrichment), or no less than about 6,380 (about 99.5% deuterium enrichment). The deuterium enrichment can be determined using conventional analytical methods known to one of ordinary skill in the art, including mass spectrometry and nuclear magnetic resonance spectroscopy. In certain embodiments, at least one of the atoms of a compound provided herein, as specified as deuterium-enriched, has deuterium enrichment of no less than about 50%, no less than about 70%, no less than about 80%, no less than about 90%, or no less than about 98%.

In certain embodiments, a compound provided herein is isolated or purified. In certain embodiments, a compound provided herein has a purity of at least about 90%, at least about 95%, at least about 98%, at least about 99%, or at least about 99.5% by weight.

The compounds provided herein are intended to encompass all possible stereoisomers unless a particular stereochemistry is specified. Where a compound provided herein contains an alkenyl group, the compound may exist as one or mixture of geometric cis/trans (or Z/E) isomers. Where structural isomers are interconvertible, the compound may exist as a single tautomer or a mixture of tautomers. This can take the form of proton tautomerism in the compound that contains, for example, an imino, keto, or oxime group; or so-called valence tautomerism in the compound that contains an aromatic moiety. It follows that a single compound may exhibit more than one type of isomerism.

A compound provided herein can be enantiomerically pure, such as a single enantiomer or a single diastereomer, or be stereoisomeric mixtures, such as a mixture of enantiomers, e.g., a racemic mixture of two enantiomers; or a mixture of two or more diastereomers. As such, one of ordinary skill in the art will recognize that administration of a compound in its (R) form is equivalent, for the compound that undergoes epimerization in vivo, to administration of the compound in its (S) form. Conventional techniques for the preparation/isolation of individual enantiomers include synthesis from a suitable optically pure precursor, asymmetric synthesis from achiral starting materials, or resolution of an enantiomeric mixture, for example, chiral chromatography, recrystallization, resolution, diastereomeric salt formation, or derivatization into diastereomeric adducts followed by separation.

When a compound provided herein contains an acidic or basic moiety, it can also be provided as a pharmaceutically acceptable salt. See, Berge et al., J. Pharm. Sci. 1977, 66, 1-19; Handbook of Pharmaceutical Salts: Properties, Selection, and Use, 2nd ed.; Stahl and Wermuth Eds.; John Wiley & Sons, 2011. In certain embodiments, a pharmaceutically acceptable salt of a compound provided herein is a solvate. In certain embodiments, a pharmaceutically acceptable salt of a compound provided herein is a hydrate.

Suitable acids for use in the preparation of pharmaceutically acceptable salts of a compound provided herein include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, ca-oxoglutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (L)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts of a compound provided herein include, but are not limited to, inorganic bases, such as magnesium hydroxide, calcium hydroxide, potassium hydroxide, zinc hydroxide, and sodium hydroxide; and organic bases, such as primary, secondary, tertiary, and quaternary, aliphatic and aromatic amines, including, but not limited to, L-arginine, benethamine, benzathine, choline, deanol, diethanolamine, diethylamine, dimethylamine, dipropylamine, diisopropylamine, 2-(diethyl-amino)ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, triethanolamine, trimethylamine, triethylamine, N-methyl-D-glucamine, 2-amino-2-(hydroxymethyl)-1,3-propanediol, and tromethamine.

The disclosure will be further understood by the following non-limiting examples.

### EXAMPLES

As used herein, the symbols and conventions used in these processes, schemes and examples, regardless of whether a particular abbreviation is specifically defined, are consistent with those used in the contemporary scientific literature, for example, the Journal of the American Chemical Society, the Journal of Medicinal Chemistry, or the Journal of Biological Chemistry. Specifically, but without limitation, the following abbreviations may be used in the examples and throughout the specification: g (grams); mg (milligrams); mL (milliliters); μL (microliters); mM (millimolar); μM (micromolar); mmol (millimoles); min (minute or minutes); h (hour or hours); Bn (benzyl); Boc (tert-butoxycarbonyl); DCM (dichloromethane); DMF (dimethylformamide); DMAP (4-dimethylaminopyridine); EDCI (1-ethyl-3-(3-dimethylamino-propyl)carbodiimide); TEA (triethylamine); TFA (trifluoroacetic acid); LCMS (liquid chromatography-mass spectrometry); MS (mass spectrometry); NMR (nuclear magnetic resonance); and prep-HPLC (preparative high performance liquid chromatography).

For all of the following examples, standard work-up and purification methods known to those skilled in the art can be utilized. Unless otherwise indicated, all temperatures are expressed in ° C. (degrees Centigrade). All reactions are conducted at room temperature unless otherwise specified. Synthetic methodologies illustrated herein are intended to exemplify the applicable chemistry through the use of specific examples and are not indicative of the scope of the disclosure.

### Example 1

### Preparation of 8,8′-((2-hydroxyethyl)azanediyl)bis(N-hexyl-N-octyloctanamide) A1

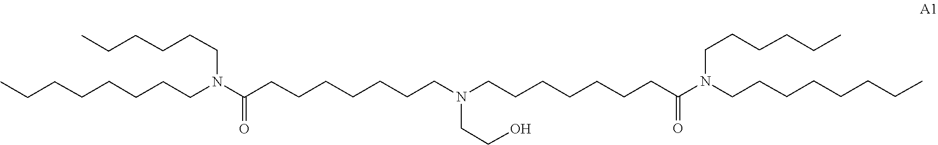 [SMILES: CCCCCCCCN(CCCCCC)C(=O)CCCCCCCN(CCO)CCCCCCCC(=O)N(CCCCCC)CCCCCCCC]

Compound A1 was prepared as shown in Scheme 1.

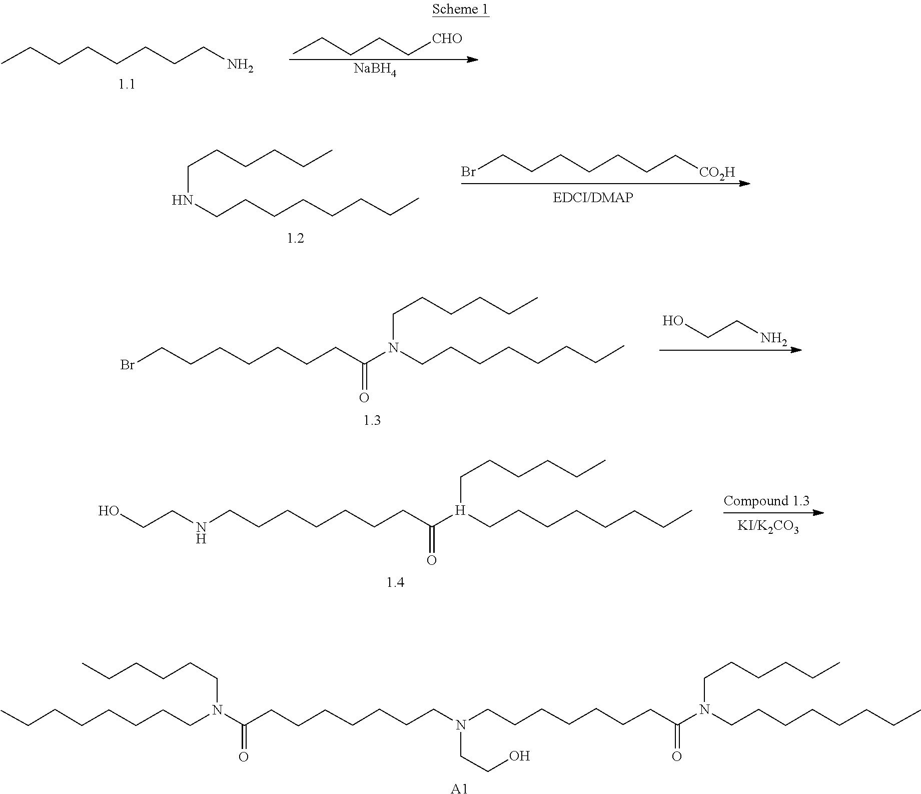

N-Hexyloctan-1-amine 1.2. A solution of 1-octanamine 1.1 (30 mL, 181 mmol) and hexanal (26.5 mL, 217 mmol) in dry ethanol (400 mL) was stirred for 6 h at room temperature, followed by addition of NaBH₄ (13.7 g, 362 mmol) at 0° C. After stirred at room temperature overnight, the reaction mixture was concentrated in vacuo and then treated with NaOH (4N, 600 mL). The mixture was stirred for 20-30 min and extracted with diethyl ether (3×200 mL). The combined organic phase was washed with water (2×100 mL) and brine (1×100 mL), dried over anhydrous MgSO₄, and concentrated in vacuo to afford compound 1.2 (39 g) in 95% yield.

8-Bromo-N-hexyl-N-octyloctanamide 1.3. To a solution of 8-bromooctanoic acid (2.2 g, 9.65 mmol) and DMAP (9 mg, 0.074 mmol) in dry DCM was added EDCI (2.9 g, 15 mmol). After the solution was stirred for 20-30 min at room temperature, compound 1.2 (1.6 g, 7.42 mmol) was added. The reaction mixture was stirred at room temperature overnight, and then diluted in DCM (150 mL), washed with water (3×50 mL), and concentrated in vacuo to yield a crude product, which was purified by silica gel column chromatography eluting with MeOH in DCM (0-10%) to afford compound 1.3 (2.6 g) in 84% yield.

N-Hexyl-8-((2-hydroxyethyl)amino)-N-octyloctanamide 1.4. A solution of compound 1.3 (0.61 g, 1.46 mmol) and ethanolamine (4.4 mL. 73 mmol) in dry DCM (3 mL) was stirred at room temperature overnight. The reaction mixture was then diluted with DCM (50 mL), washed with water (3×15 mL), dried over anhydrous MgSO₄, and concentrated in vacuo to afford compound 1.4 (0.53 g) in 91% yield.

8,8′-((2-Hydroxyethyl)azanediyl)bis(N-hexyl-N-octyloctanamide) A1. A mixture of compound 1.3 (0.84 g, 2 mmol), compound 1.4 (0.53 g, 1.33 mmol), K₂CO₃ (0.37 g, 2.66 mmol), and KI (a catalytic amount) in dry DMF (5 mL) was stirred at 70° C. overnight. The reaction mixture was then diluted with DCM (50 mL), washed with water (3×15 mL), dried over anhydrous MgSO₄, and concentrated in vacuo to yield a crude product, which was purified by silica gel column chromatography eluting with MeOH in DCM (0-10%) to afford compound A1 (0.64 g) in 66% yield. ¹H NMR (300 MHz, CDCl₃) δ 3.67 (s, 2H), 3.23 (dt, J=25.7, 7.5 Hz, 8H), 2.70 (d, J=38.1 Hz, 6H), 2.27 (t, J=7.5 Hz, 4H), 1.74-1.40 (m, 16H), 1.29 (dd, J=12.9, 7.1 Hz, 44H), 0.88 (h, J=3.4 Hz, 12H).

### Example 2

### Preparation of ((2-hydroxyethyl)azanediyl)bis(hexane-6,1-diyl) bis(hexyl(octyl)carbamate) A2

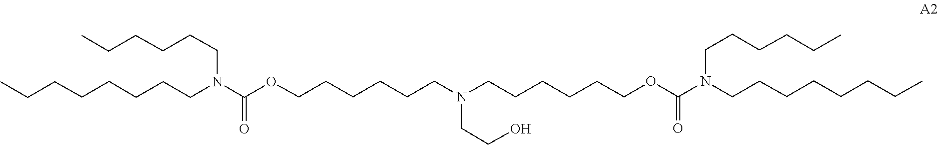 [SMILES: CCCCCCCCN(CCCCCC)C(=O)OCCCCCCN(CCO)CCCCCCOC(=O)N(CCCCCC)CCCCCCCC]

Compound A2 is prepared as shown in Scheme 2.

6-Bromohexyl (4-nitrophenyl)carbonate 2.2. To a solution of 6-bromo-1-hexanol 2.1 (2.77 g, 15.3 mmol) and pyridine (2.3 mL, 29 mmol) in dry DCM (10 mL) was added nitrophenyl chloroformate (6.2 g, 31 mmol) at 0° C. After stirred at room temperature overnight, the reaction mixture was diluted with DCM (100 mL) and washed with water (3×30 mL). The combined organic phase was filtered through silica and the concentrated to afford compound 2.2, which was used directly in the next step without further purification.

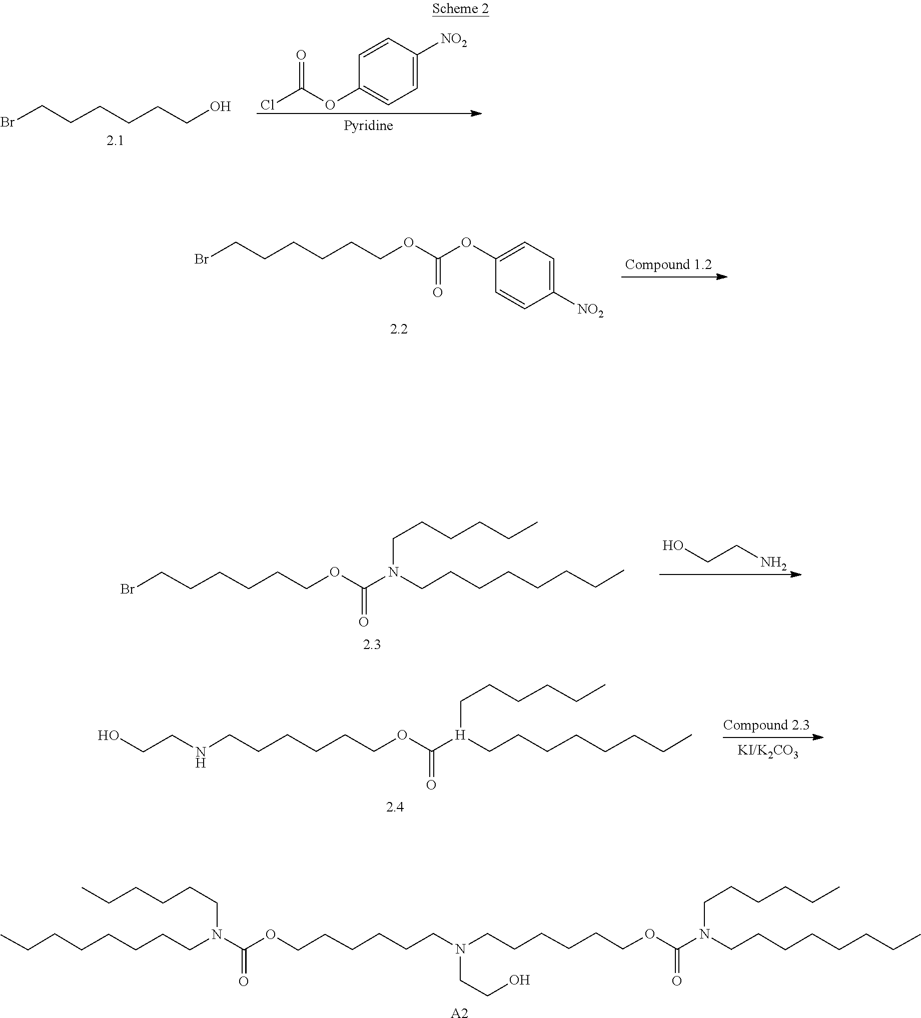

6-Bromohexyl hexyl(octyl)carbamate 2.3. A solution of compound 1.2 (0.78 g, 3.7 mmol) and compound 2.2 (1.32 g, 3.8 mmol) in dry DMF (10 mL) was stirred at 50° C. The reaction mixture was concentrated and then filtered through silica to yield compound 2.3, which was used directly in the next step without further purification.

6-((2-Hydroxyethyl)amino)hexyl hexyl(octyl)carbamate 2.4. A solution of compound 2.3 (2.63 g, 12.3 mmol) and ethanolamine (337 mL, 61.6 mmol) in dry DCM (10 mL) was stirred at room temperature overnight. The reaction mixture was then diluted with DCM (50 mL) and washed with water (3×20 mL). The organic phase was concentrated to yield a crude product, which was purified by silica gel column chromatography eluting with MeOH in DCM to afford compound 2.4 (0.64 g). ¹H NMR (300 MHz, CDCl₃) δ 4.05 (t, J=6.6 Hz, 2H), 3.72 (t, J=5.1 Hz, 2H), 3.17 (s, 4H), 2.86 (t, J=5.1 Hz, 2H), 2.71 (t, J=7.3 Hz, 2H), 2.37 (s, 2H), 1.73-1.16 (m, 28H), 1.01-0.71 (m, 6H).

((2-Hydroxyethyl)azanediyl)bis(hexane-6,1-diyl) bis(hexyl(octyl)carbamate) A2. Compound A2 is prepared from compound 2.4 according to the procedures described in Example 1 for compound A1.

### Example 3

### Preparation of N-(2-hydroxyethyl)-N,N-di(7-(pentadecan-7-oxycarbonyloxy)heptyl)amine A3

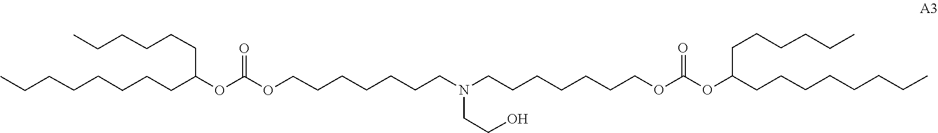 [SMILES: CCCCCCCCC(CCCCCC)OC(=O)OCCCCCCCN(CCO)CCCCCCCOC(=O)OC(CCCCCC)CCCCCCCC]

Compound A3 is prepared as shown in Scheme 3.

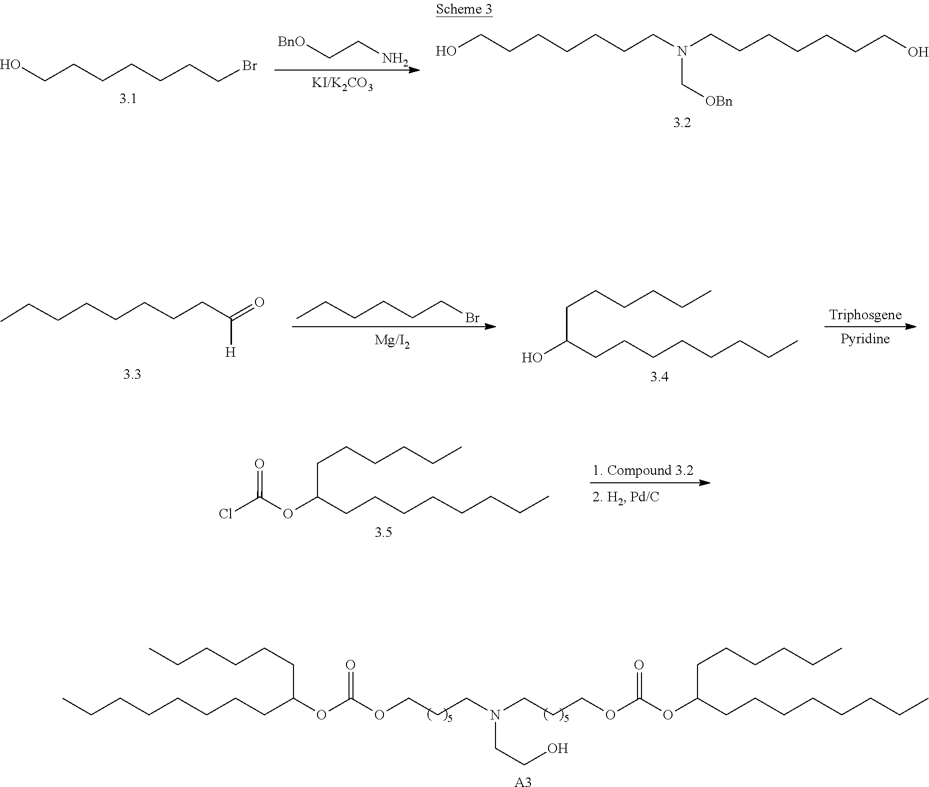

### Example 4

### Preparation of ((((2-hydroxyethyl)azanediyl)bis(ethane-2,1-diyl))bis(oxy))bis(ethane-2,1-diyl) bis(2-hexyldecanoate) A4

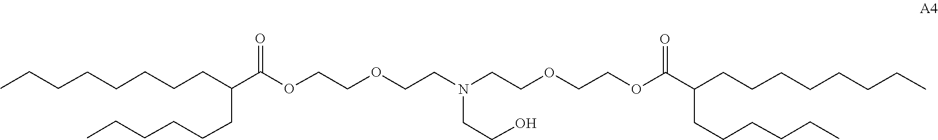 [SMILES: CCCCCCCCC(CCCCCC)C(=O)OCCOCCN(CCO)CCOCCOC(=O)C(CCCCCC)CCCCCCCC]

Compound A4 was prepared as shown in Scheme 4.

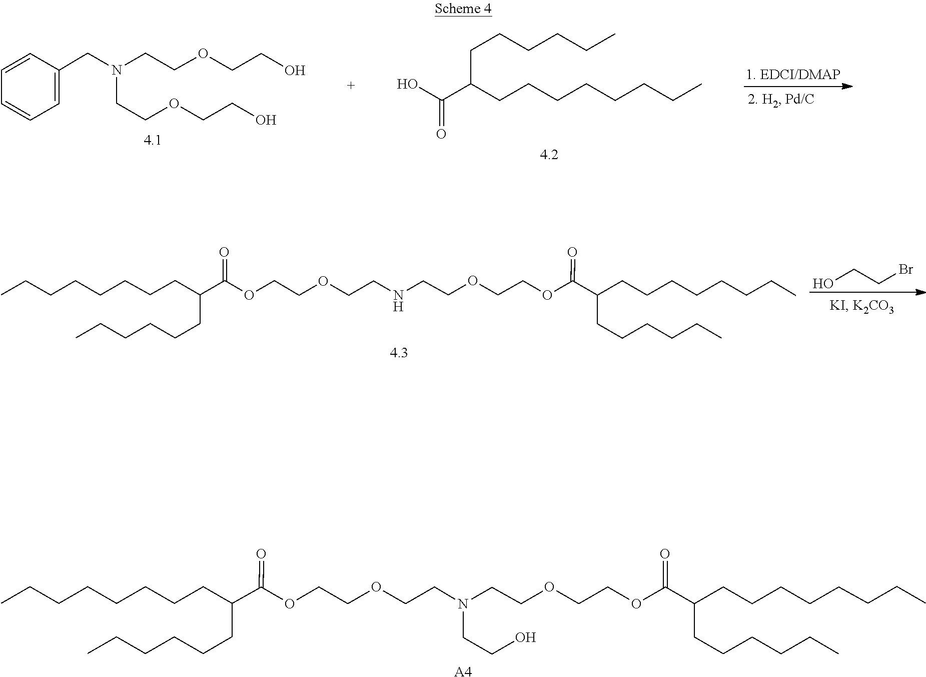

((Azanediylbis(ethane-2,1-diyl))bis(oxy))bis(ethane-2,1-diyl) bis(2-hexyl-decanoate) 4.3. To a solution of compound 4.2 (2.3 g, 8.82 mmol), DMAP (0.22 g, 1.77 mmol), and TEA (2.5 mL, 18 mmol) in dry DCM (20 mL) was added EDCI (2 g, 11 mmol). The mixture was stirred for 30 min at room temperature. Compound 4.1 (1 g, 3.53 mmol) was then added. After stirred at room temperature overnight, the reaction mixture was washed with 1N HCl, water, dried over anhydrous MgSO₄, and concentrated to yield protected compound 4.3, which was deprotected with H₂ in the presence of Pd/C (500 mg) in ethanol (150 mL) overnight. The reaction mixture was filtered through celite and concentrated to afford compound 4.3 (2.67 g), which was used directly in the next step without further purification.

((((2-Hydroxyethyl)azanediyl)bis(ethane-2,1-diyl))bis(oxy))bis(ethane-2,1-diyl) bis(2-hexyldecanoate) A4. A mixture of compound 4.3 (0.65 g, 0.97 mmol), 2-bromoethanol (6.1 g, 49 mmol), KI (a catalytic amount), and K₂CO₃ (0.54 g, 3.88 mmol) in dry DMF (3 mL) was stirred at 70° C. overnight. The reaction mixture was concentrated, diluted with water, and extracted with DCM. The combined organic phase was concentrated to yield a crude product, which was purified by silica gel column chromatography eluting with MeOH in DCM to afford compound A4 (0.56 g) in 81% yield. ¹H NMR (300 MHz, CDCl₃) δ 4.22 (d, J=5.1 Hz, 4H), 3.88-3.44 (m, 16H), 2.42-2.25 (m, 2H), 1.57-1.25 (s, 48H), 0.97-0.82 (m, 12H).

### Example 5

### Preparation of di(heptadecan-9-yl) 10-(2-hydroxyethyl)-4,7,13,16-tetraoxa-10-azanonadecanedioate A5

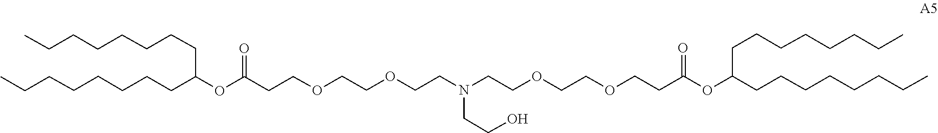 [SMILES: CCCCCCCCC(CCCCCCCC)OC(=O)CCOCCOCCN(CCO)CCOCCOCCC(=O)OC(CCCCCCCC)CCCCCCCC]

Compound A5 was prepared as shown in Scheme 5.

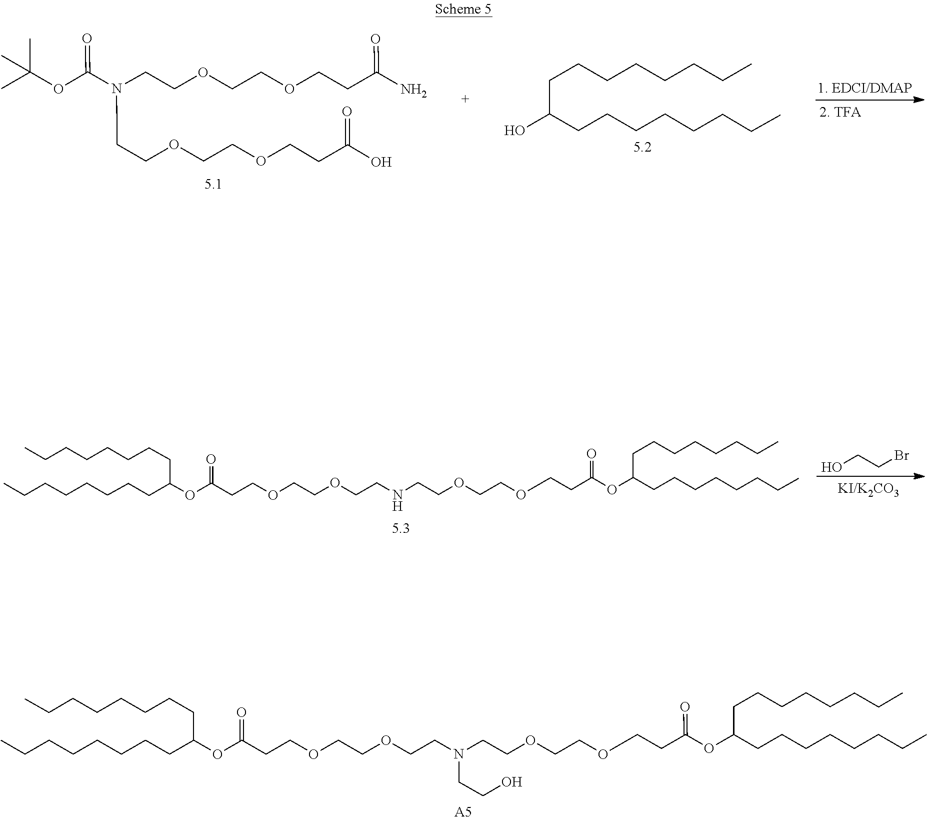

Di(heptadecan-9-yl) 4,7,13,16-tetraoxa-10-azanonadecanedioate 5.3. To a mixture of compound 5.1 (0.5 g, 1.14 mmol), DMAP (35 mg, 0.29 mmol), and TEA (0.48 mL, 3.43 mmol) in dry DCM (10 mL) was added EDCI (0.33 g, 1.71 mmol). After the solution was stirred for 30 min at room temperature, compound 5.2 (0.73 g, 2.86 mmol) was added. The reaction mixture was stirred at room temperature overnight. The reaction mixture was then washed with 1N HCl, water, dried over anhydrous MgSO₄, and concentrated to yield a crude protected compound, which was purified by silica gel column chromatography eluting with using MeOH in DCM to afford a protected compound 5.3 (0.84 g) in 81% yield. The protected compound was deprotected with TFA (10 mL) in DCM (10 mL) overnight. The reaction mixture was washed with H₂O, dried over anhydrous MgSO₄, and concentrated to afford compound 5.3 (0.81 g), which was used directly in the next step without further purification.

Di(heptadecan-9-yl) 10-(2-hydroxyethyl)-4,7,13,16-tetraoxa-10-azanonadecane-dioate A5. A mixture of compound 5.3 (0.81 g, 0.1 mmol), 2-bromoethanol (2.49 g, 20 mmol), KI (a catalytic amount), and K₂CO₃ (0.55 g, 3.98 mmol) in dry DMF (5 mL) was stirred at 70° C. The reaction mixture was concentrated, diluted with water, and extracted with DCM. The organic phase was concentrated to yield a crude product, which was purified by silica gel column chromatography eluting with MeOH in DCM to afford compound A5 (0.41 g) in 75% yield. ¹H NMR (300 MHz, CDCl₃) δ 4.87 (t, J=6.2 Hz, 2H), 3.73 (t, J=6.6 Hz, 8H), 3.60 (s, 8H), 3.10 (s, 6H), 2.57 (t, J=6.5 Hz, 4H), 1.50 (d, J=6.8 Hz, 8H), 1.25 (s, 48H), 0.97-0.80 (m, 12H).

### Example 6

### Preparation of bis(1,1,1,15,15,15-hexafluoropentadecan-7-yl) 7,7′-((2-hydroxyethyl)-azanediyl)diheptanoate A6

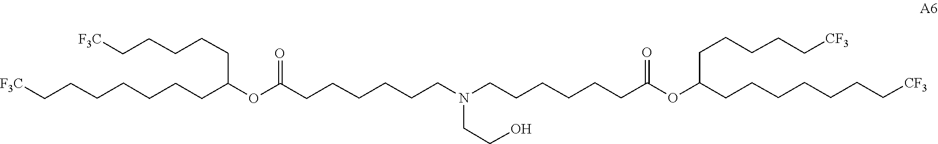 [SMILES: O=C(CCCCCCN(CCO)CCCCCCC(=O)OC(CCCCCCCC(F)(F)F)CCCCCC(F)(F)F)OC(CCCCCCCC(F)(F)F)CCCCCC(F)(F)F]

Compound A6 is prepared as shown in Scheme 6.

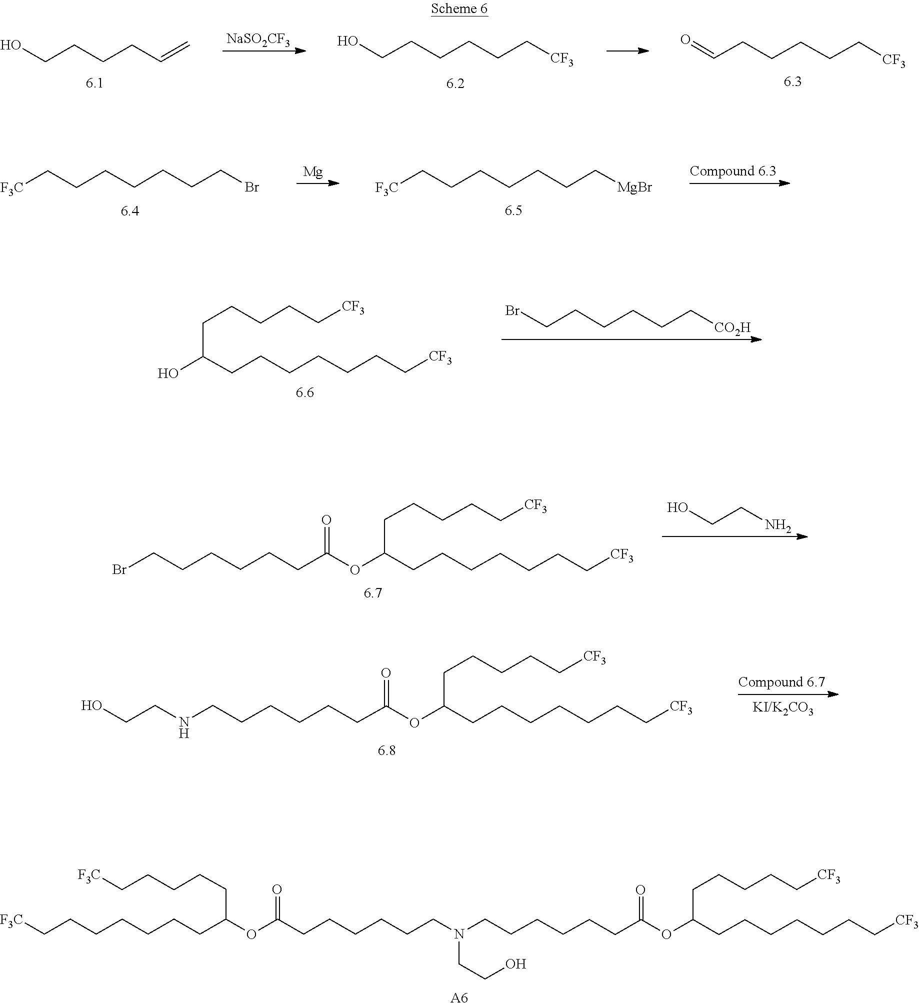

### Example 7

### Preparation of 1,1,1,15,15,15-hexafluoropentadecan-7-yl 8-((2-hydroxyethyl)(6-oxo-6-((11,11,11-trifluoroundecyl)oxy)hexyl)amino)octanoate A7

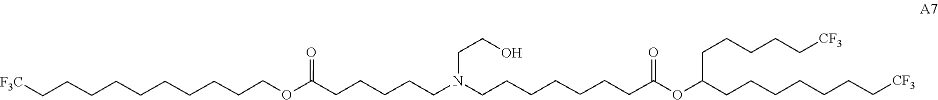 [SMILES: O=C(CCCCCN(CCO)CCCCCCCC(=O)OC(CCCCCCCC(F)(F)F)CCCCCC(F)(F)F)OCCCCCCCCCCC(F)(F)F]

Compound A7 is prepared as shown in Scheme 7.

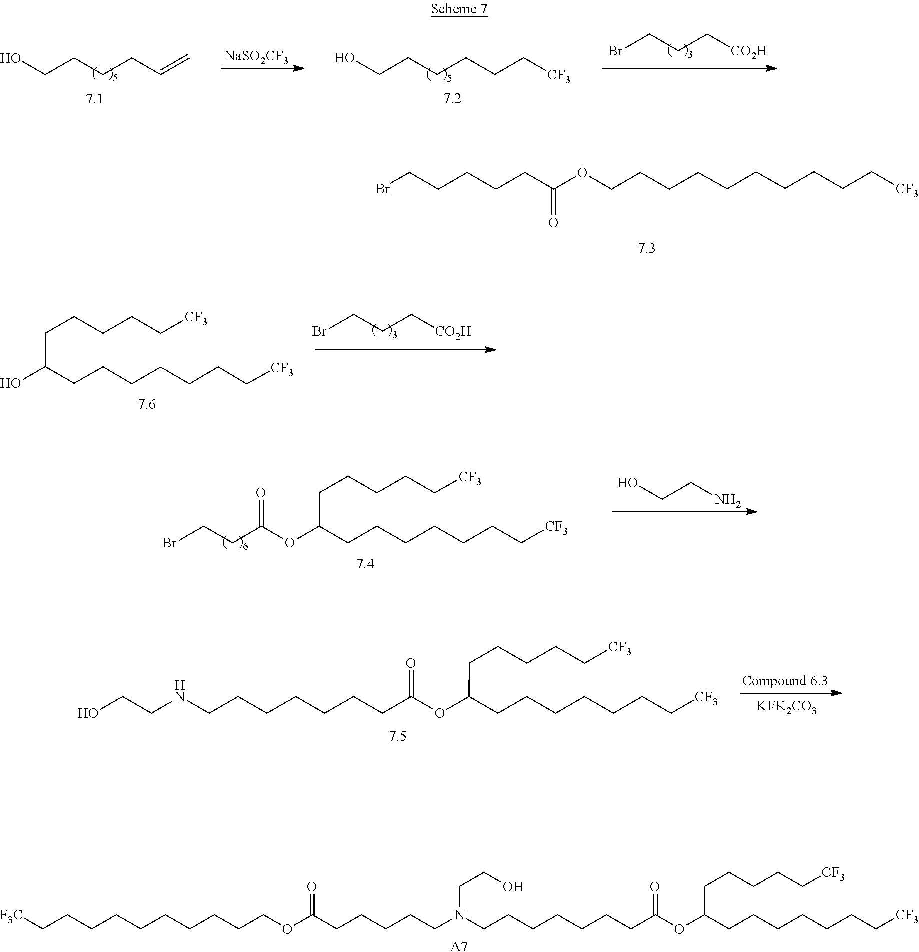

### Example 8

### Preparation of heptadecan-9-yl 8-((2-hydroxyethyl)(6-oxo-6-((11,11,11-trifluoroundecyl)oxy)-hexyl)amino)octanoate A10

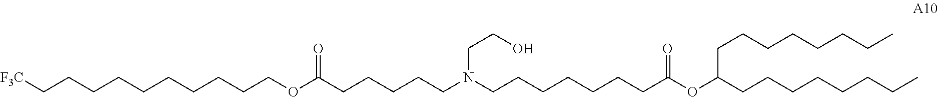 [SMILES: CCCCCCCCC(CCCCCCCC)OC(=O)CCCCCCCN(CCO)CCCCCC(=O)OCCCCCCCCCCC(F)(F)F]

Compound A10 was prepared as shown in Scheme 8.

11,11,11-Trifluoroundecan-1-ol 8.2. To a mixture of NaSO₂CF₃ (2 g, 12.8 mmol) and Mn(OAc)₃·H₂O (6.9 g, 26 mmol) under Ar was added compound 8.1 (2 g, 12.8 mmol), followed by addition of acetic acid (150 mL). After stirred for 24 h at room temperature, the reaction mixture was poured into a solution containing Na₂S₂O₄ (15 g) in water (800 mL). Na₂CO₃ was then added slowly until bubbles stopped forming (pH ˜7). The reaction mixture was extracted with DCM, washed with H₂O, dried over anhydrous MgSO₄, and concentrated to afford compound 8.2, which was used directly in the next reaction without further purification.

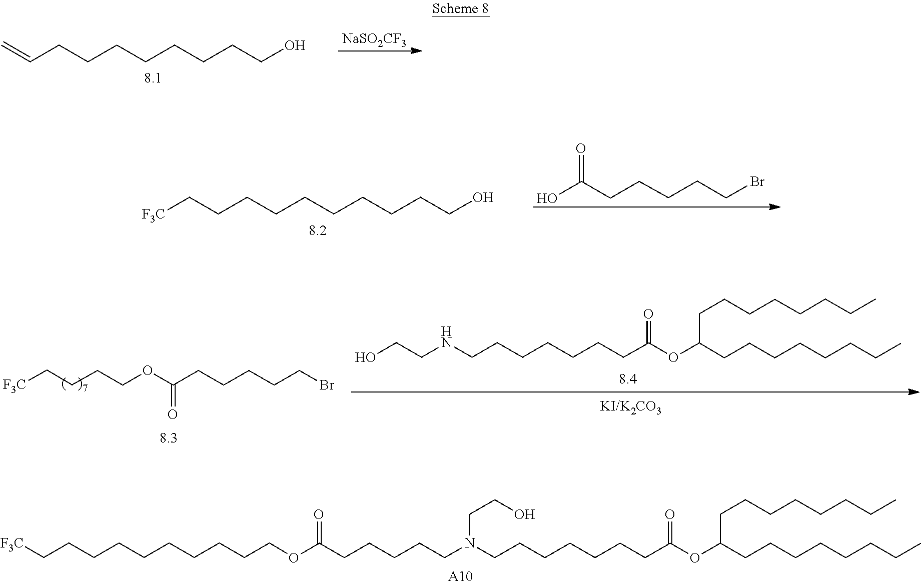

11,11,11-Trifluoroundecyl 6-bromohexanoate 8.3. To a solution of 6-bromo-hexanoic acid (0.62 g, 3.18 mmol), DMAP (65 mg, 0.63 mmol), and TEA (0.74 mL, 5.30 mmol) in dry DCM (15 mL) was added EDCI (0.71 g, 3.71 mmol). The mixture was stirred for 30 min at room temperature and compound 8.2 (0.6 g, 2.65 mmol). After stirred at room temperature overnight, the reaction mixture was washed with 1N HCl, water, dried over anhydrous MgSO₄, and concentrated to afford compound 8.3, which was used directly in the next reaction without further purification.

Heptadecan-9-yl 8-((2-hydroxyethyl)(6-oxo-6-((11,11,11-trifluoroundecyl)oxy)-hexyl)amino)octanoate A10. A mixture of compound 8.3 (0.52 g, 1.29 mmol), compound 8.4 (0.47 g, 1.07 mmol), KI (a catalytic amount), and K₂CO₃ (0.22 g, 1.61 mmol) in dry DMF (3 mL) was stirred overnight at 70° C. After concentrated, the reaction mixture was diluted with water and extracted with DCM. The organic phase was concentrated to yield a crude product, which was purified by silica gel column chromatography eluting with MeOH in DCM to afford compound A10 (0.3 g) in 37% yield. ¹H NMR (300 MHz, CDCl₃) δ 4.84 (p, J=6.3 Hz, 1H), 4.04 (t, J=6.8 Hz, 2H), 3.84 (t, J=4.9 Hz, 2H), 2.98 (t, J=5.0 Hz, 2H), 2.86 (dt, J=8.5, 4.3 Hz, 4H), 2.28 (dt, J=12.1, 7.4 Hz, 4H), 2.03 (dd, J=9.2, 6.7 Hz, 2H), 1.85-1.10 (m, 62H), 0.97-0.71 (m, 6H); ¹⁹F NMR (282 MHz, CDCl₃) δ −66.39.

The following compounds were prepared similarly according to the synthetic procedures or methodologies exemplified herein.

Heptadecan-9-yl (6-((3-hydroxypropyl)(6-(((undecyloxy)carbonyl)amino)hexyl)-amino)hexyl)carbamate A46. ¹H NMR (300 MHz, CDCl₃) δ 4.84 (m, 1H), 4.72 (m, 2H), 4.01 (t, J=6.80 Hz, 2H), 3.77 (t, J=6.80 Hz, 2H), 3.15 (m, 6H), 2.67 (t, J=6.72 Hz, 2H), 2.44 (m, 4H), 1.62-1.33 (m, 12H), 1.32-1.18 (m, 50H), 0.86 (t, J=6.74 Hz, 9H); MS (ESI) m z: 754.1 [M+H]⁺.

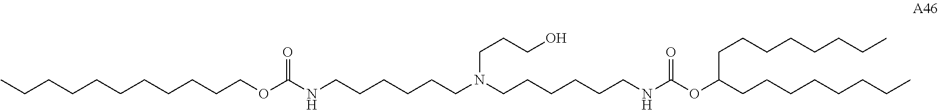 [SMILES: CCCCCCCCCCCOC(=O)NCCCCCCN(CCCO)CCCCCCNC(=O)OC(CCCCCCCC)CCCCCCCC]

N,N-(((4-Hydroxybutyl)azanediyl)bis(hexane-6,1-diyl))bis(2-hexyldecanamide) A48. ¹H NMR (300 MHz, CDCl₃) δ 5.69 (t, J=4.89 Hz, 2H), 3.55 (m, 2H), 3.23 (m, 4H), 2.46 (m, 6H), 1.51 (m, 2H), 1.62-1.40 (m, 12H), 1.39-1.16 (m, 58H), 0.87 (t, J=6.83 Hz, 12H).

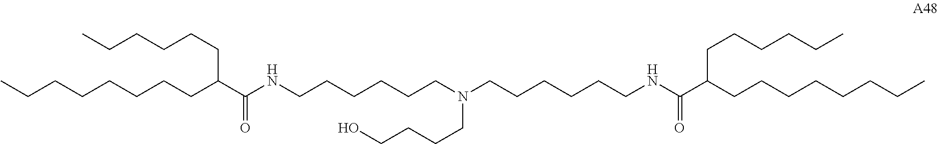 [SMILES: CCCCCCCCC(CCCCCC)C(=O)NCCCCCCN(CCCCO)CCCCCCNC(=O)C(CCCCCC)CCCCCCCC]

N,N-(((2-Hydroxyethyl)azanediyl)bis(hexane-6,1-diyl))bis(2-hexyldecanamide) A49. ¹H NMR (300 MHz, CDCl₃) δ 5.59 (t, J=4.98 Hz, 2H), 3.55 (m, 2H), 3.25 (m, 4H), 2.60 (m, 2H), 2.44 (m, 4H), 1.89 (m, 2H), 1.70-1.38 (m, 14H), 1.38-1.16 (m, 40H), 0.87 (t, J=6.88 Hz 12H).

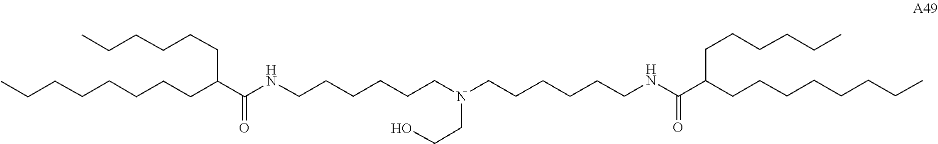 [SMILES: CCCCCCCCC(CCCCCC)C(=O)NCCCCCCN(CCO)CCCCCCNC(=O)C(CCCCCC)CCCCCCCC]

Heptadecan-9-yl 6-((2-hydroxyethyl)(5-(nonyloxy)-5-oxopentyl)amino)hexanoate B1. ¹H NMR (300 MHz, CDCl₃) b 4.86 (m, 1H), 4.06 (t, J=6.81 Hz, 2H), 3.58 (m, 2H), 2.65 (m, 2H), 2.53 (m, 4H), 2.29 (m, 4H), 1.79-1.40 (m, 18H), 1.39-1.12 (m, 36H), 0.88 (t, J=6.80 Hz, 9H).

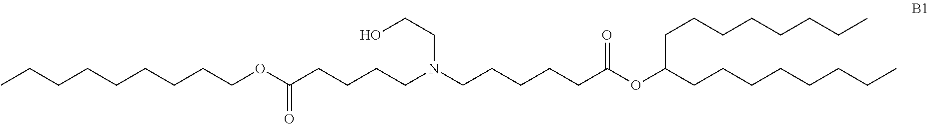 [SMILES: CCCCCCCCCOC(=O)CCCCN(CCO)CCCCCC(=O)OC(CCCCCCCC)CCCCCCCC]

Heptadecan-9-yl 6-((6-(heptyloxy)-6-oxohexyl)(2-hydroxyethyl)amino)hexanoate B2. ¹H NMR (300 MHz, CDCl₃) δ 4.83 (m, 1H), 4.08 (t, J=6.72 Hz, 2H), 2.98-2.43 (m, 6H), 2.33 (m, 4H), 1.74-1.50 (m, 16H), 1.50-1.24 (m, 36H), 0.90 (t, J=6.81 Hz, 9H).

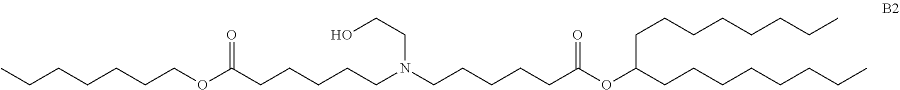 [SMILES: CCCCCCCCC(CCCCCCCC)OC(=O)CCCCCN(CCO)CCCCCC(=O)OCCCCCCC]

Heptadecan-9-yl 6-((2-hydroxyethyl)(6-oxo-6-(undecyloxy)hexyl)amino)-hexanoate B3. ¹H NMR (300 MHz, CDCl₃) δ 4.86 (m, 1H), 4.05 (t, J=6.74 Hz, 2H), 3.51 (t, J=6.68 Hz, 2H), 2.56 (t, J=6.70 Hz, 2H), 2.44 (t, J=6.78 Hz, 4H), 2.28 (m, 4H), 1.63 (m, 6H), 1.48 (m, 4H), 1.46-1.22 (m, 48H), 0.88 (t, J=6.82 Hz, 9H).

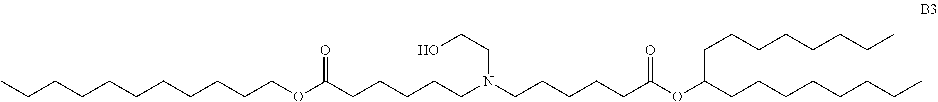 [SMILES: CCCCCCCCCCCOC(=O)CCCCCN(CCO)CCCCCC(=O)OC(CCCCCCCC)CCCCCCCC]

Heptadecan-9-yl 7-((7-(heptyloxy)-7-oxoheptyl)(2-hydroxyethyl)amino)-heptanoate B4. ¹H NMR (300 MHz, CDCl₃) δ 4.86 (m, 1H), 4.05 (t, J=6.74 Hz, 2H), 3.51 (t, J=6.70 Hz, 2H), 2.55 (t, J=6.69 Hz, 2H), 2.42 (m, 4H), 2.28 (m, 4H), 1.64 (m, 6H), 1.49 (m, 6H), 1.46-1.22 (m, 42H), 0.86 (t, J=6.80 Hz, 9H).

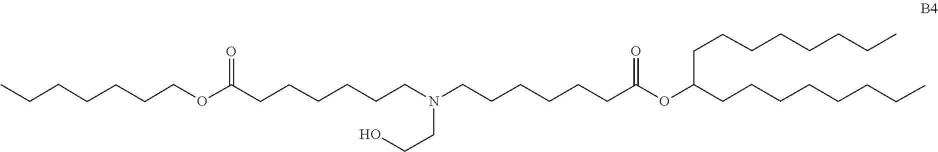 [SMILES: CCCCCCCCC(CCCCCCCC)OC(=O)CCCCCCN(CCO)CCCCCCC(=O)OCCCCCCC]

Heptadecan-9-yl 7-((2-hydroxyethyl)(7-(nonyloxy)-7-oxoheptyl)amino)-heptanoate B5. ¹H NMR (300 MHz, CDCl₃) δ 4.86 (m, 1H), 4.05 (t, J=6.76 Hz, 2H), 3.51 (t, J=6.71 Hz, 2H), 2.56 (t, J=6.69 Hz, 2H), 2.43 (m, 4H), 2.28 (m, 4H), 1.62 (m, 6H), 1.48 (m, 6H), 1.46-1.19 (m, 44H), 0.87 (t, J=6.82 Hz, 9H).

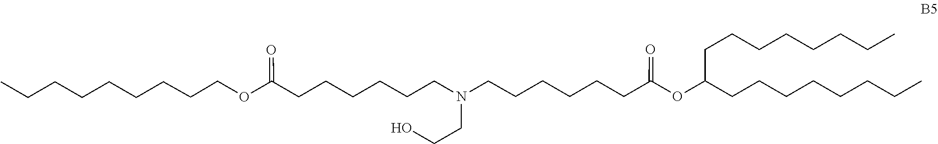 [SMILES: CCCCCCCCCOC(=O)CCCCCCN(CCO)CCCCCCC(=O)OC(CCCCCCCC)CCCCCCCC]

Heptadecan-9-yl 7-((2-hydroxyethyl)(7-oxo-7-(undecyloxy)heptyl)amino)-heptanoate B6. ¹H NMR (300 MHz, CDCl₃) δ 4.86 (m, 1H), 4.05 (t, J=6.76 Hz, 2H), 3.59 (t, J=4.96 Hz, 2H), 2.66 (t, J=4.98 Hz, 2H), 2.53 (m, 4H), 2.29 (m, 4H), 1.70-1.49 (m, 14H), 1.48-1.17 (m, 46H), 0.87 (t, J=6.82 Hz, 9H).

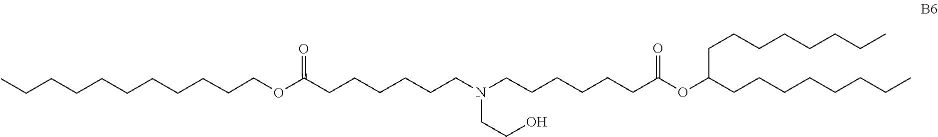 [SMILES: CCCCCCCCCCCOC(=O)CCCCCCN(CCO)CCCCCCC(=O)OC(CCCCCCCC)CCCCCCCC]

Heptadecan-9-yl 8-((8-(heptyloxy)-8-oxooctyl)(2-hydroxyethyl)amino)octanoate B7. ¹H NMR (300 MHz, CDCl₃) δ 4.84 (m, 1H), 4.03 (t, J=6.84 Hz, 2H), 3.51 (t, J=5.98 Hz, 2H), 2.57 (t, J=5.98 Hz, 2H), 2.44 (m, 4H), 2.28 (m, 4H), 1.67-1.40 (m, 12H), 1.38-1.17 (m, 44H), 0.85 (t, J=6.80 Hz, 9H).

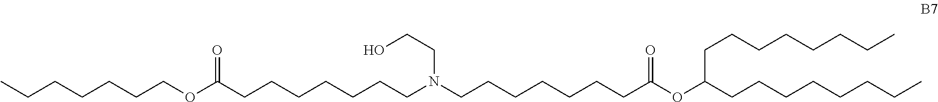 [SMILES: CCCCCCCCC(CCCCCCCC)OC(=O)CCCCCCCN(CCO)CCCCCCCC(=O)OCCCCCCC]

Heptadecan-9-yl 8-((2-hydroxyethyl)(8-oxo-8-(undecyloxy)octyl)amino)-octanoate B8. ¹H NMR (300 MHz, CDCl₃) δ 4.86 (m, 1H), 4.05 (t, J=6.80 Hz, 2H), 3.63 (m, 2H), 2.75-2.29 (m, 6H), 2.28 (m, 4H), 1.68-1.40 (m, 16H), 1.38-1.17 (m, 50H), 0.88 (t, J=6.78 Hz, 9H).

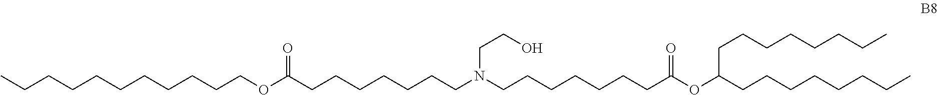 [SMILES: CCCCCCCCCCCOC(=O)CCCCCCCN(CCO)CCCCCCCC(=O)OC(CCCCCCCC)CCCCCCCC]

Heptadecan-9-yl 7-((2-hydroxyethyl)(6-oxo-6-(undecyloxy)hexyl)amino)-heptanoate B9. ¹H NMR (300 MHz, CDCl₃) δ 4.86 (m, 1H), 4.05 (t, J=6.80 Hz, 2H), 3.57 (m, 2H), 2.63 (m, 2H), 2.50 (m, 4H), 2.28 (m, 4H), 1.71-1.41 (m, 14H), 1.38-1.16 (m, 44H), 0.86 (t, J=6.80 Hz, 9H).

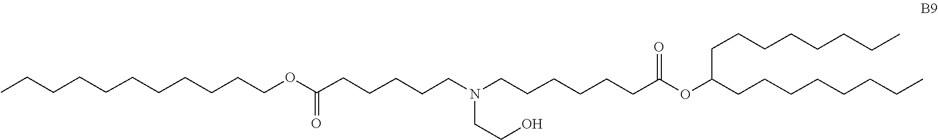 [SMILES: CCCCCCCCCCCOC(=O)CCCCCN(CCO)CCCCCCC(=O)OC(CCCCCCCC)CCCCCCCC]

Heptadecan-9-yl 8-((6-(heptyloxy)-6-oxohexyl)(2-hydroxyethyl)amino)octanoate B10. ¹H NMR (300 MHz, CDCl₃) δ 4.88 (m, 1H), 4.05 (t, J=6.81 Hz, 2H), 3.67 (m, 2H), 2.75 (m, 2H), 2.62 (m, 4H), 2.30 (m, 4H), 1.75-1.46 (m, 12H), 1.43-1.13 (m, 40H), 0.88 (t, J=6.80 Hz, 9H).

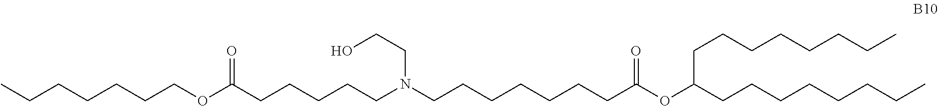 [SMILES: CCCCCCCCC(CCCCCCCC)OC(=O)CCCCCCCN(CCO)CCCCCC(=O)OCCCCCCC]

Heptadecan-9-yl 8-((2-hydroxyethyl)(6-(nonyloxy)-6-oxohexyl)amino)octanoate B11. ¹H NMR (300 MHz, CDCl₃) δ 4.68 (m, 1H), 4.05 (t, J=6.81 Hz, 2H), 3.62 (m, 2H), 2.77-2.36 (m, 6H), 2.28 (m, 4H), 1.75-1.46 (m, 16H), 1.43-1.14 (m, 42H), 0.88 (t, J=6.81 Hz, 9H).

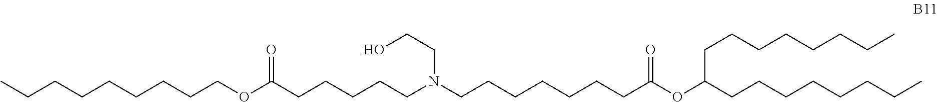 [SMILES: CCCCCCCCCOC(=O)CCCCCN(CCO)CCCCCCCC(=O)OC(CCCCCCCC)CCCCCCCC]

Heptyl 7-((6-(heptadecan-9-yloxy)-6-oxohexyl)(2-hydroxyethyl)amino)-heptanoate B12. ¹H NMR (300 MHz, CDCl₃) δ 4.86 (m, 1H), 4.05 (t, J=6.84 Hz, 2H), 3.52 (t, J=5.98 Hz, 2H), 2.57 (t, J=6.00 Hz, 2H), 2.46 (m, 4H), 2.28 (m, 4H), 1.75-1.42 (m, 14H), 1.42-1.20 (m, 38H), 0.87 (t, J=6.80 Hz, 9H).

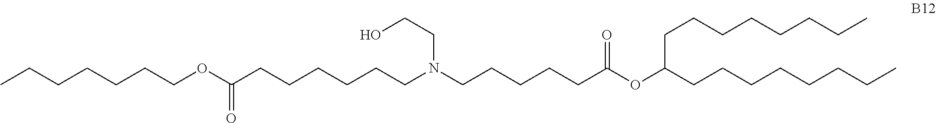 [SMILES: CCCCCCCCC(CCCCCCCC)OC(=O)CCCCCN(CCO)CCCCCCC(=O)OCCCCCCC]

Undecyl 7-((6-(heptadecan-9-yloxy)-6-oxohexyl)(2-hydroxyethyl)amino)-heptanoate B13. ¹H NMR (300 MHz, CDCl₃) δ 4.88 (m, 1H), 4.07 (t, J=6.80 Hz, 2H), 3.55 (t, J=6.04 Hz, 2H), 2.58 (t, J=6.00 Hz, 2H), 2.48 (m, 4H), 2.30 (m, 4H), 1.64 (m, 6H), 1.51 (m, 8H), 1.45-1.20 (m, 46H), 0.89 (t, J=6.80 Hz, 9H).

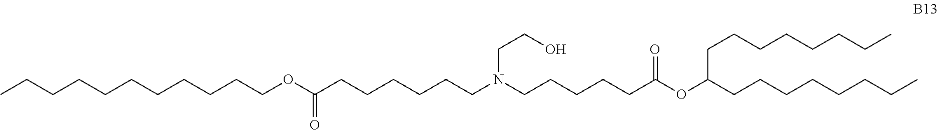 [SMILES: CCCCCCCCCCCOC(=O)CCCCCCN(CCO)CCCCCC(=O)OC(CCCCCCCC)CCCCCCCC]

Heptyl 8-((6-(heptadecan-9-yloxy)-6-oxohexyl)(2-hydroxyethyl)amino)octanoate B14. ¹H NMR (300 MHz, CDCl₃) δ 4.86 (m, 1H), 4.05 (t, J=6.83 Hz, 2H), 3.51 (t, J=6.00 Hz, 2H), 2.56 (t, J=6.02 Hz, 2H), 2.45 (m, 4H), 2.28 (m, 4H), 1.63 (m, 6H), 1.49 (m, 4H), 1.45-1.20 (m, 44H), 0.86 (t, J=6.78 Hz, 9H).

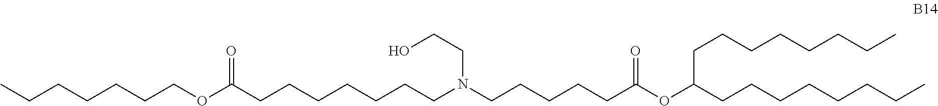 [SMILES: CCCCCCCCC(CCCCCCCC)OC(=O)CCCCCN(CCO)CCCCCCCC(=O)OCCCCCCC]

Nonyl 8-((6-(heptadecan-9-yloxy)-6-oxohexyl)(2-hydroxyethyl)amino)octanoate B15. ¹H NMR (300 MHz, CDCl₃) δ 4.86 (m, 1H), 4.05 (t, J=6.83 Hz, 2H), 3.61 (m, 2H), 2.68 (m, 2H), 2.55 (m, 4H), 2.29 (t, J=6.79 Hz, 4H), 1.72-1.46 (m, 12H), 1.45-1.20 (m, 44H), 0.87 (t, J=6.83 Hz, 9H).

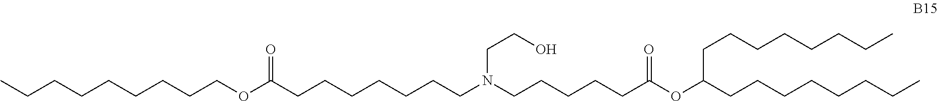 [SMILES: CCCCCCCCCOC(=O)CCCCCCCN(CCO)CCCCCC(=O)OC(CCCCCCCC)CCCCCCCC]

Undecyl 8-((6-(heptadecan-9-yloxy)-6-oxohexyl)(2-hydroxyethyl)amino)-octanoate B16. ¹H NMR (300 MHz, CDCl₃) δ 4.85 (m, 1H), 4.04 (t, J=6.83 Hz, 2H), 3.91 (m, 2H), 3.15-2.80 (m, 6H), 2.29 (m, 4H), 1.74-1.46 (m, 16H), 1.45-1.20 (m, 46H), 0.87 (t, J=6.80 Hz, 9H).

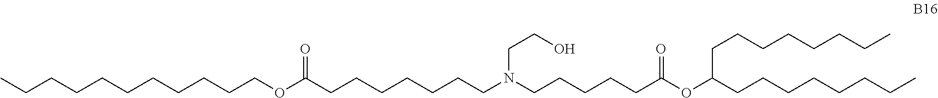 [SMILES: CCCCCCCCCCCOC(=O)CCCCCCCN(CCO)CCCCCC(=O)OC(CCCCCCCC)CCCCCCCC]

Nonyl 8-((7-(heptadecan-9-yloxy)-7-oxoheptyl)(2-hydroxyethyl)amino)octanoate B17. ¹H NMR (300 MHz, CDCl₃) δ 4.86 (m, 1H), 4.05 (t, J=6.81 Hz, 2H), 3.68 (m, 2H), 3.10-2.60 (m, 6H), 2.29 (m, 4H), 1.73-1.46 (m, 14H), 1.45-1.19 (m, 44H), 0.87 (t, J=6.80 Hz, 9H).

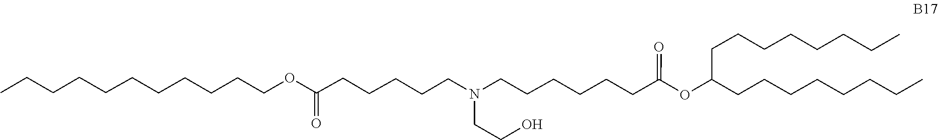 [SMILES: CCCCCCCCCCCOC(=O)CCCCCN(CCO)CCCCCCC(=O)OC(CCCCCCCC)CCCCCCCC]

Heptadecan-9-yl 10-((2-hydroxyethyl)(8-(nonyloxy)-8-oxooctyl)amino)decanoate B18. ¹H NMR (300 MHz, CDCl₃) δ 4.86 (m, 1H), 4.05 (t, J=6.79 Hz, 2H), 3.79 (m, 2H), 2.93-2.75 (m, 6H), 2.27 (m, 4H), 1.75-1.46 (m, 16H), 1.45-1.20 (m, 50H), 0.85 (t, J=6.82 Hz, 9H).

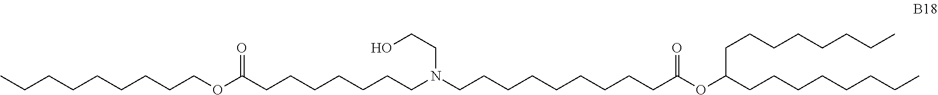 [SMILES: CCCCCCCCCOC(=O)CCCCCCCN(CCO)CCCCCCCCCC(=O)OC(CCCCCCCC)CCCCCCCC]

Heptadecan-9-yl 10-((2-hydroxyethyl)(10-oxo-10-(undecyloxy)decyl)amino)-decanoate B19. ¹H NMR (300 MHz, CDCl₃) δ 4.89 (m, 1H), 4.08 (t, J=6.84 Hz, 2H), 3.66 (m, 2H), 2.80-2.51 (m, 6H), 2.31 (m, 4H), 1.63 (m, 8H), 1.53 (m, 6H), 1.46-1.18 (m, 60H), 0.90 (t, J=6.79 Hz, 9H).

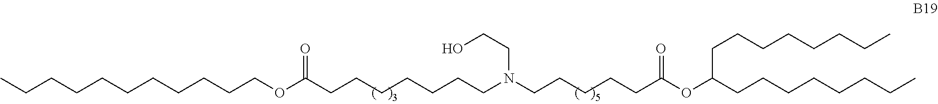

Heptadecan-9-yl 6-((6-(heptyloxy)-6-oxohexyl)(3-hydroxypropyl)amino)-hexanoate B20. ¹H NMR (300 MHz, CDCl₃) (4.90 (m, 1H), 4.05 (t, J=6.68 Hz, 2H), 3.78 (t, J=6.17 Hz, 2H), 2.63 (t, J=6.0 Hz, 2H), 2.41 (m, 4H), 2.31 (m, 4H), 1.72-1.53 (m, 9H), 1.51-1.43 (m, 4H), 1.32-1.14 (m, 40H), 0.87 (t, J=6.82 Hz, 9H).

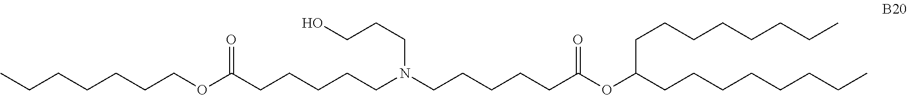 [SMILES: CCCCCCCCC(CCCCCCCC)OC(=O)CCCCCN(CCCO)CCCCCC(=O)OCCCCCCC]

Heptadecan-9-yl 6-((3-hydroxypropyl)(6-(nonyloxy)-6-oxohexyl)amino)-hexanoate B21. ¹H NMR (300 MHz, CDCl₃) δ 4.86 (m, 1H), 4.05 (t, J=6.80 Hz, 2H), 3.81 (t, J=6.24 Hz, 2H), 2.86 (m, 2H), 2.67 (m, 4H), 2.31 (m, 4H), 1.79-1.46 (m, 20H), 1.45-1.18 (m, 36H), 0.87 (t, J=6.79 Hz, 9H).

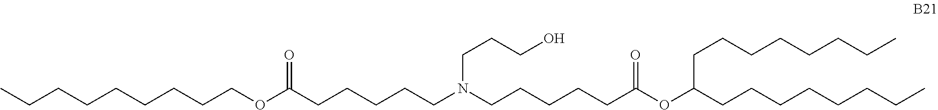 [SMILES: CCCCCCCCCOC(=O)CCCCCN(CCCO)CCCCCC(=O)OC(CCCCCCCC)CCCCCCCC]

Heptadecan-9-yl 7-((7-(heptyloxy)-7-oxoheptyl)(3-hydroxypropyl)amino)-heptanoate B22. ¹H NMR (300 MHz, CDCl₃) δ 4.85 (m, 1H), 4.05 (t, J=6.82 Hz, 2H), 3.82 (t, J=6.20 Hz, 2H), 3.09 (m, 2H), 2.90 (m, 4H), 2.28 (m, 4H), 1.92 (m, 2H), 1.78-1.12 (m, 54H), 0.86 (t, J=6.79 Hz, 9H).

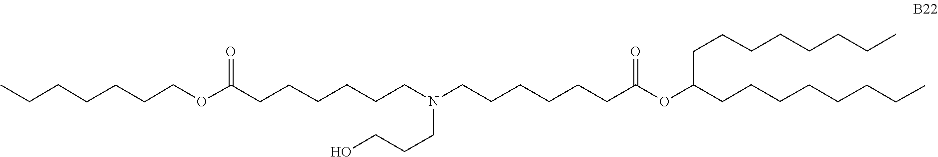 [SMILES: CCCCCCCCC(CCCCCCCC)OC(=O)CCCCCCN(CCCO)CCCCCCC(=O)OCCCCCCC]

Heptadecan-9-yl 8-((3-hydroxypropyl)(8-(nonyloxy)-8-oxooctyl)amino)octanoate B23. ¹H NMR (300 MHz, CDCl₃) δ 4.85 (m, 1H), 4.05 (t, J=6.82 Hz, 2H), 3.81 (t, J=6.31 Hz, 2H), 3.06 (m, 2H), 2.80 (m, 4H), 2.27 (m, 4H), 1.91 (m, 2H), 1.75-1.47 (m, 16H), 1.44-1.20 (m, 46H), 0.85 (t, J=6.83 Hz, 9H).

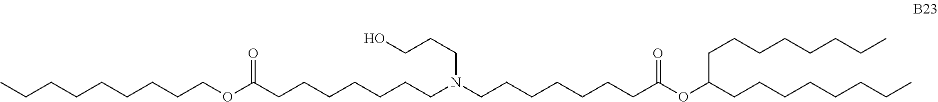 [SMILES: CCCCCCCCCOC(=O)CCCCCCCN(CCCO)CCCCCCCC(=O)OC(CCCCCCCC)CCCCCCCC]

Heptadecan-9-yl 8-((3-hydroxypropyl)(8-oxo-8-(undecyloxy)octyl)amino)-octanoate B24. ¹H NMR (300 MHz, CDCl₃) δ 4.85 (m, 1H), 4.04 (t, J=6.82 Hz, 2H), 3.81 (t, J=6.22 Hz, 2H), 3.04 (m, 2H), 2.86 (m, 4H), 2.26 (m, 4H), 1.93 (m, 2H), 1.75-1.47 (m, 14H), 1.45-1.08 (m, 52H), 0.87 (t, J=6.80 Hz, 9H).

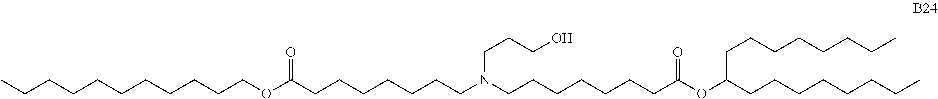 [SMILES: CCCCCCCCCCCOC(=O)CCCCCCCN(CCCO)CCCCCCCC(=O)OC(CCCCCCCC)CCCCCCCC]

Heptadecan-9-yl 7-((3-hydroxypropyl)(6-(nonyloxy)-6-oxohexyl)amino)-heptanoate B25. ¹H NMR (300 MHz, CDCl₃) δ 4.85 (m, 1H), 4.05 (t, J=6.83 Hz, 2H), 3.82 (t, J=6.28 Hz, 2H), 3.01 (m, 2H), 2.85 (m, 4H), 2.30 (m, 4H), 1.77-1.47 (m, 18H), 1.40-1.18 (m, 40H), 0.87 (t, J=6.79 Hz, 9H).

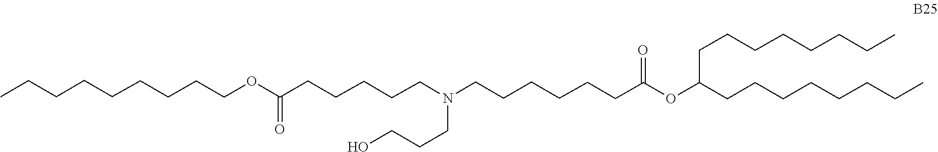 [SMILES: CCCCCCCCCOC(=O)CCCCCN(CCCO)CCCCCCC(=O)OC(CCCCCCCC)CCCCCCCC]

Heptadecan-9-yl 8-((6-(heptyloxy)-6-oxohexyl)(3-hydroxypropyl)amino)-octanoate B26. ¹H NMR (300 MHz, CDCl₃) δ 4.86 (m, 1H), 4.06 (t, J=6.86 Hz, 2H), 3.80 (t, J=6.28 Hz, 2H), 2.79 (m, 2H), 2.59 (m, 4H), 2.30 (m, 4H), 1.75-1.33 (m, 18H), 1.31-1.18 (m, 38H), 0.88 (t, J=6.81 Hz, 9H).

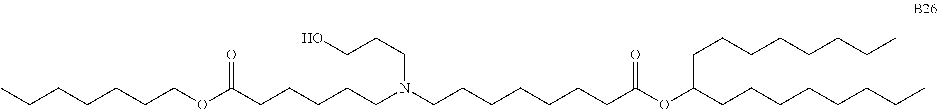 [SMILES: CCCCCCCCC(CCCCCCCC)OC(=O)CCCCCCCN(CCCO)CCCCCC(=O)OCCCCCCC]

Heptadecan-9-yl 8-((3-hydroxypropyl)(6-oxo-6-(undecyloxy)hexyl)amino)-octanoate B27. ¹H NMR (300 MHz, CDCl₃) δ 4.86 (m, 1H), 4.05 (t, J=6.78 Hz, 2H), 3.80 (t, J=6.36 Hz, 2H), 2.74 (m, 2H), 2.52 (m, 4H), 2.28 (m, 4H), 1.78-1.46 (m, 20H), 1.44-1.18 (m, 44H), 0.88 (t, J=6.83 Hz, 9H).

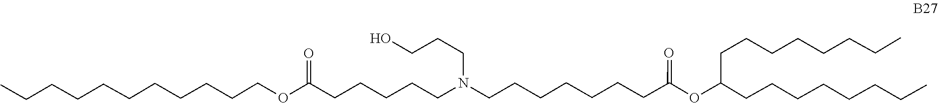 [SMILES: CCCCCCCCCCCOC(=O)CCCCCN(CCCO)CCCCCCCC(=O)OC(CCCCCCCC)CCCCCCCC]

Di(heptadecan-9-yl) 6,6′-((2-hydroxyethyl)azanediyl)dihexanoate B28. ¹H NMR (300 MHz, CDCl₃) δ 4.88 (m, 2H), 3.56 (t, 0.1=6.70 Hz, 2H), 2.62 (t, J=6.42 Hz, 2H), 2.50 (t, J=6.64 Hz, 4H), 2.30 (t, J=6.42 Hz, 4H), 1.66 (m, 4H), 1.57-1.36 (m, 12H), 1.36-1.20 (m, 52H), 0.90 (t, J=6.79 Hz, 12H).

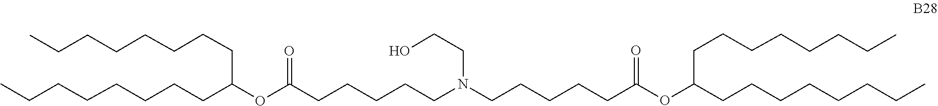 [SMILES: CCCCCCCCC(CCCCCCCC)OC(=O)CCCCCN(CCO)CCCCCC(=O)OC(CCCCCCCC)CCCCCCCC]

Di(heptadecan-9-yl) 7,7′-((2-hydroxyethyl)azanediyl)diheptanoate B29. ¹H NMR (300 MHz, CDCl₃) δ 4.88 (m, 2H), 3.58 (t, J=6.54 Hz, 2H), 2.64 (t, J=6.02 Hz, 2H), 2.49 (t, J=6.64 Hz, 4H), 2.30 (t, J=6.71 Hz, 4H), 1.62 (m, 4H), 1.58-1.39 (m, 10H), 1.36-1.20 (m, 58H), 0.90 (t, J=6.75 Hz, 12H).

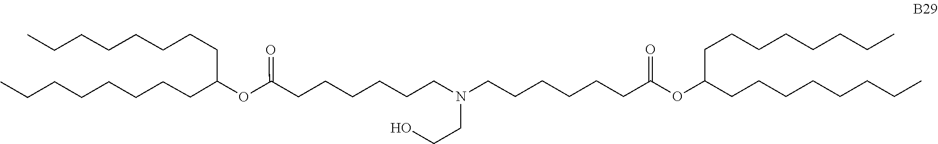 [SMILES: CCCCCCCCC(CCCCCCCC)OC(=O)CCCCCCN(CCO)CCCCCCC(=O)OC(CCCCCCCC)CCCCCCCC]

((2-Hydroxyethyl)azanediyl)bis(hexane-6,1-diyl) bis(2-hexyldecanoate) B30. ¹H NMR (300 MHz, CDCl₃) Q 4.06 (t, J=6.82 Hz, 4H), 3.59 (m, 2H), 2.65 (m 2H), 2.53 (m, 4H), 2.31 (m, 2H), 1.91 (m, 2H), 1.75-1.50 (m, 10H), 1.48-1.30 (m, 12H), 1.30-1.11 (m, 40H), 0.87 (t, J=7.02 Hz, 12H).

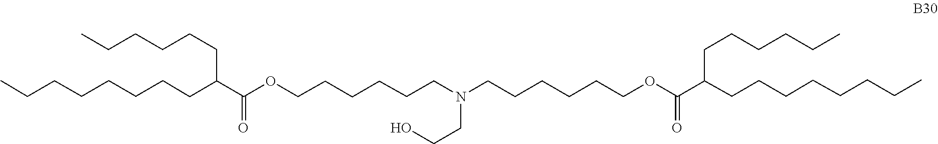 [SMILES: CCCCCCCCC(CCCCCC)C(=O)OCCCCCCN(CCO)CCCCCCOC(=O)C(CCCCCC)CCCCCCCC]

7-((2-Hydroxyethyl)(8-oxo-8-(pentadecan-7-yloxy)octyl)amino)heptyl 2-hexyldecanoate B31. ¹H NMR (300 MHz, CDCl₃) Q 4.06 (t, J=6.80 Hz, 4H), 3.60 (m, 2H), 2.67 (m 2H), 2.54 (m, 4H), 2.29 (m, 2H), 1.70-1.09 (m, 68H), 0.87 (t, J=6.90 Hz, 12H).

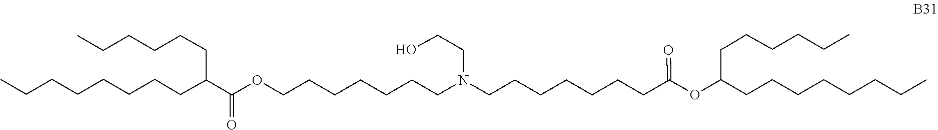 [SMILES: CCCCCCCCC(CCCCCC)OC(=O)CCCCCCCN(CCO)CCCCCCCOC(=O)C(CCCCCC)CCCCCCCC]

((2-Hydroxyethyl)azanediyl)bis(octane-8,1-diyl) bis(2-hexyldecanoate) B32. ¹H NMR (300 MHz, CDCl₃) Q 4.06 (t, J=6.80 Hz, 4H), 3.62 (m, 2H), 2.82-2.32 (m, 6H), 2.29 (m, 2H), 1.75-1.53 (m, 20H), 1.43 (m, 4H), 1.42-1.20 (m, 50H), 0.87 (t, J=6.96 Hz, 12H).

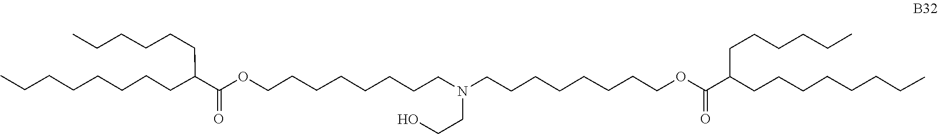 [SMILES: CCCCCCCCC(CCCCCC)C(=O)OCCCCCCCCN(CCO)CCCCCCCCOC(=O)C(CCCCCC)CCCCCCCC]

((3-Hydroxypropyl)azanediyl)bis(hexane-6,1-diyl) bis(2-hexyldecanoate) B33. ¹H NMR (300 MHz, CDCl₃) Q 4.05 (t, J=6.83 Hz, 4H), 3.79 (t, J=6.21 Hz, 2H), 2.70 (t, J=6.23 Hz, 2H), 2.27 (m, 4H), 2.28 (m, 2H), 1.72 (m, 2H), 1.69-1.49 (m, 12H), 1.49-1.28 (m, 12H), 1.28-1.18 (m, 41H), 0.86 (t, J=6.98 Hz, 12H).

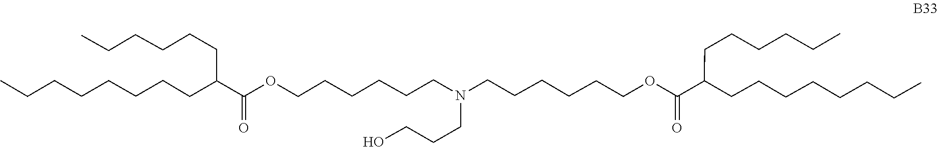 [SMILES: CCCCCCCCC(CCCCCC)C(=O)OCCCCCCN(CCCO)CCCCCCOC(=O)C(CCCCCC)CCCCCCCC]

((3-Hydroxypropyl)azanediyl)bis(heptane-7,1-diyl) bis(2-hexyldecanoate) B34. ¹H NMR (300 MHz, CDCl₃) Q 4.06 (t, J=6.79 Hz, 4H), 3.79 (t, J=6.74 Hz, 2H), 2.63 (t, J=6.79 Hz, 2H), 2.38 (m, 4H), 2.32 (m 2H), 1.73-1.50 (m, 10H), 1.50-32-1.30 (m, 8H), 1.30-1.20 (m, 54H), 0.87 (t, J=6.98 Hz, 12H).

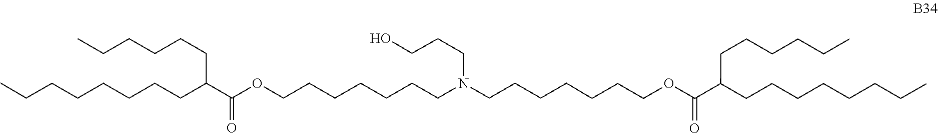 [SMILES: CCCCCCCCC(CCCCCC)C(=O)OCCCCCCCN(CCCO)CCCCCCCOC(=O)C(CCCCCC)CCCCCCCC]

((3-Hydroxypropyl)azanediyl)bis(octane-8,1-diyl) bis(2-hexyldecanoate) B35. ¹H NMR (300 MHz, CDCl₃) Q 4.06 (t, J=6.86 Hz, 4H), 3.82 (t, J=6.38 Hz, 2H), 3.12-2.50 (m, 6H), 2.32 (m, 2H), 1.87 (m, 2H), 1.69-1.33 (m, 20H), 1.31-1.13 (m, 52H), 0.87 (t, J=6.98 Hz, 12H).

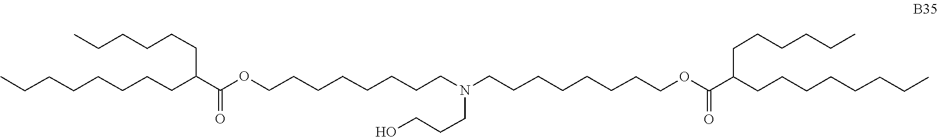 [SMILES: CCCCCCCCC(CCCCCC)C(=O)OCCCCCCCCN(CCCO)CCCCCCCCOC(=O)C(CCCCCC)CCCCCCCC]

((4-Hydroxybutyl)azanediyl)bis(heptane-7,1-diyl) bis(2-hexyldecanoate) B36. ¹H NMR (300 MHz, CDCl₃) Q 4.05 (t, J=6.82 Hz, 4H), 3.57 (m, 2H), 2.52 (m, 6H), 2.30 (m 2H), 1.68 (m, 4H), 1.66-1.50 (m, 12H), 1.50-1.18 (m, 56H), 0.87 (t, J=6.98 Hz, 12H).

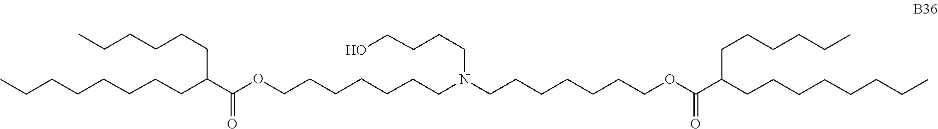 [SMILES: CCCCCCCCC(CCCCCC)C(=O)OCCCCCCCN(CCCCO)CCCCCCCOC(=O)C(CCCCCC)CCCCCCCC]

((4-Hydroxybutyl)azanediyl)bis(octane-8,1-diyl) bis(2-hexyldecanoate) B37. ¹H NMR (300 MHz, CDCl₃) Q 4.05 (t, J=6.80 Hz, 4H), 3.56 (m, 2H), 2.89-2.38 (m, 6H), 2.28 (m, 2H), 1.75-1.52 (m, 22H), 1.48 (m, 4H), 1.43-1.18 (m, 50H), 0.86 (t, J=6.92 Hz, 12H).

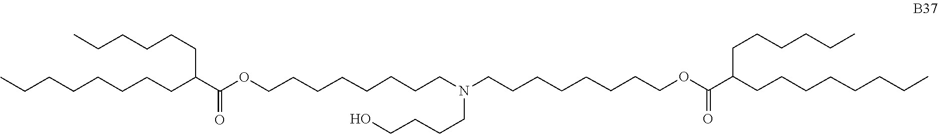 [SMILES: CCCCCCCCC(CCCCCC)C(=O)OCCCCCCCCN(CCCCO)CCCCCCCCOC(=O)C(CCCCCC)CCCCCCCC]

((5-Hydroxypentyl)azanediyl)bis(hexane-6,1-diyl) bis(2-hexyldecanoate) B38. ¹H NMR (300 MHz, CDCl₃) Q 4.05 (t, J=6.82 Hz, 4H), 3.67 (m, 2H), 2.98 (m, 6H), 2.28 (m, 2H), 1.90-1.75 (m, 2H), 1.68-1.53 (m, 12H), 1.50-1.28 (m, 14H), 1.27-1.06 (m, 42H), 0.87 (t, J=6.98 Hz, 12H).

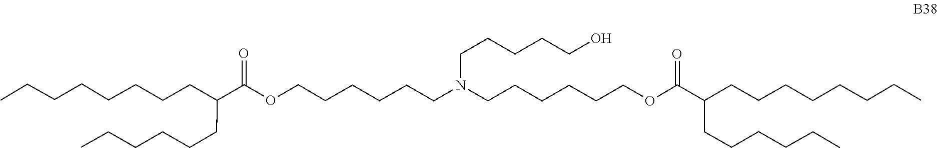 [SMILES: CCCCCCCCC(CCCCCC)C(=O)OCCCCCCN(CCCCCO)CCCCCCOC(=O)C(CCCCCC)CCCCCCCC]

((5-Hydroxypentyl)azanediyl)bis(heptane-7,1-diyl) bis(2-hexyldecanoate) B39. ¹H NMR (300 MHz, CDCl₃) Q 4.06 (t, J=6.80 Hz, 4H), 3.64 (t, J=6.69 Hz, 2H), 2.44 (m, 6H), 2.30 (m, 2H), 1.75-1.36 (m, 20H), 1.36-1.19 (m, 44H), 0.87 (t, J=6.98 Hz, 12H).

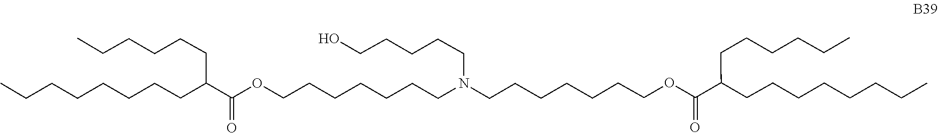 [SMILES: CCCCCCCCC(CCCCCC)C(=O)OCCCCCCCN(CCCCCO)CCCCCCCOC(=O)C(CCCCCC)CCCCCCCC]

((5-Hydroxypentyl)azanediyl)bis(octane-8,1-diyl) bis(2-hexyldecanoate) B40. ¹H NMR (300 MHz, CDCl₃) Q 4.06 (t, J=6.84 Hz, 4H), 3.66 (t, J=6.72 Hz, 2H), 2.82-2.41 (m, 6H), 2.30 (m, 2H), 1.73-1.51 (m, 22H), 1.43 (m, 4H), 1.40-1.19 (m, 52H), 0.87 (t, J=7.02 Hz, 12H).

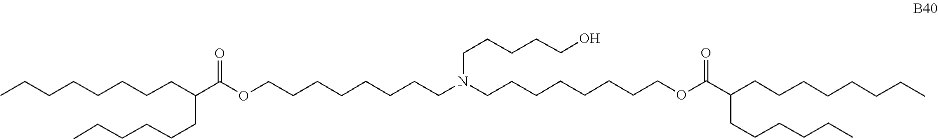 [SMILES: CCCCCCCCC(CCCCCC)C(=O)OCCCCCCCCN(CCCCCO)CCCCCCCCOC(=O)C(CCCCCC)CCCCCCCC]

Di(heptadecan-9-yl) 8,8′-((2-hydroxyethyl)azanediyl)dioctanoate B41. MS (ESI) m z: 822.6 [M+H]⁺.

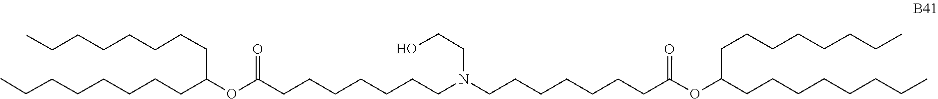 [SMILES: CCCCCCCCC(CCCCCCCC)OC(=O)CCCCCCCN(CCO)CCCCCCCC(=O)OC(CCCCCCCC)CCCCCCCC]

Heptadecan-9-yl (Z)-8-((2-hydroxyethyl)(6-(non-2-en-1-yloxy)-6-oxohexyl)-amino)octanoate B44. MS (ESI) m z: 680.2 [M+H]⁺.

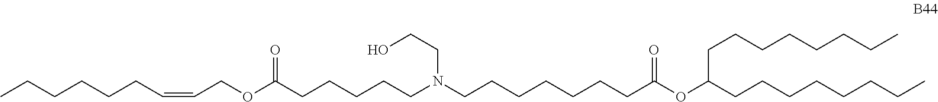 [SMILES: CCCCCC/C=C\COC(=O)CCCCCN(CCO)CCCCCCCC(=O)OC(CCCCCCCC)CCCCCCCC]

((2-(2-Methoxyethoxy)ethyl)azanediyl)bis(hexane-6,1-diyl) bis(2-hexyldecanoate) B46. MS (ESI) m z: 796.7 [M+H]⁺.

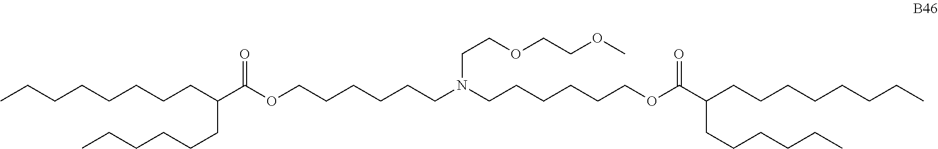 [SMILES: CCCCCCCCC(CCCCCC)C(=O)OCCCCCCN(CCCCCCOC(=O)C(CCCCCC)CCCCCCCC)CCOCCOC]

Di(pentadecan-7-yl) 8,8′-((2-hydroxyethyl)azanediyl)dioctanoate B47. MS (ESI) m z: 766.8 [M+H]⁺.

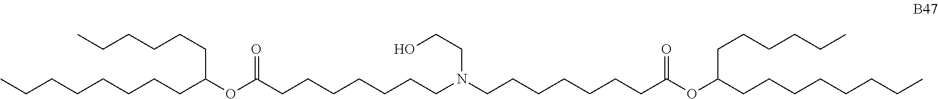 [SMILES: CCCCCCCCC(CCCCCC)OC(=O)CCCCCCCN(CCO)CCCCCCCC(=O)OC(CCCCCC)CCCCCCCC]

The following compounds are prepared similarly according to the synthetic procedures or methodologies exemplified herein.

Pentadecan-7-yl 8-((2-hydroxyethyl)(6-oxo-6-((11,11,11-trifluoroundecyl)oxy)-hexyl)amino)octanoate A8.

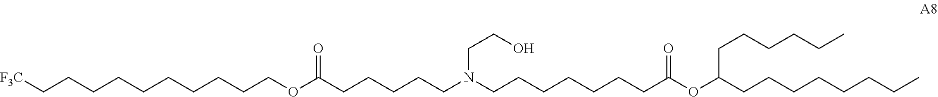 [SMILES: CCCCCCCCC(CCCCCC)OC(=O)CCCCCCCN(CCO)CCCCCC(=O)OCCCCCCCCCCC(F)(F)F]

1,1,1,17,17,17-Hexafluoroheptadecan-9-yl 8-((2-hydroxyethyl)(6-oxo-6-((11,11,11-trifluoroundecyl)oxy)hexyl)amino)octanoate A9.

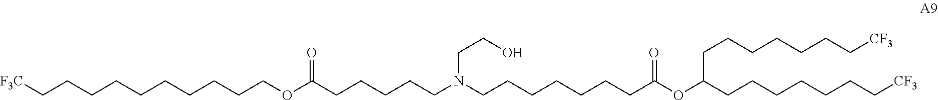 [SMILES: O=C(CCCCCN(CCO)CCCCCCCC(=O)OC(CCCCCCCC(F)(F)F)CCCCCCCC(F)(F)F)OCCCCCCCCCCC(F)(F)F]

8,8′-((3-Hydroxypropyl)azanediyl)bis(N-hexyl-N-octyloctanamide) A11.

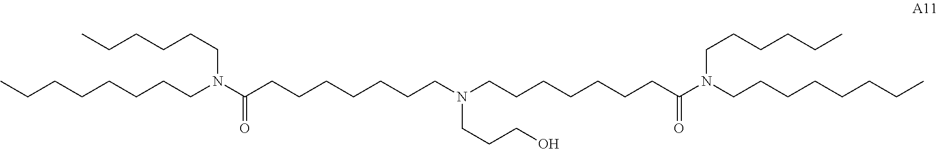 [SMILES: CCCCCCCCN(CCCCCC)C(=O)CCCCCCCN(CCCO)CCCCCCCC(=O)N(CCCCCC)CCCCCCCC]

((3-Hydroxypropyl)azanediyl)bis(hexane-6,1-diyl) bis(hexyl(octyl)carbamate) A12.

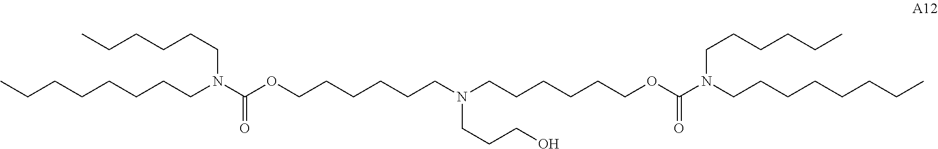 [SMILES: CCCCCCCCN(CCCCCC)C(=O)OCCCCCCN(CCCO)CCCCCCOC(=O)N(CCCCCC)CCCCCCCC]

N-(3-Hydroxypropyl)-N,N-di(7-(pentadecan-7-oxycarbonyloxy)heptyl)amine A13.

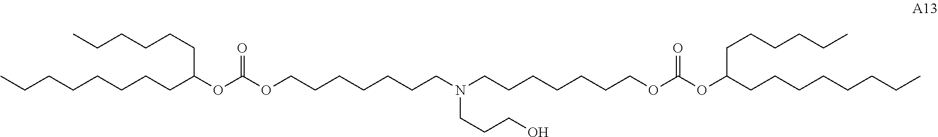 [SMILES: CCCCCCCCC(CCCCCC)OC(=O)OCCCCCCCN(CCCO)CCCCCCCOC(=O)OC(CCCCCC)CCCCCCCC]

((((3-Hydroxypropyl)azanediyl)bis(propane-3,1-diyl))bis(oxy))bis(ethane-2,1-diyl)bis(2-hexyldecanoate) A14.

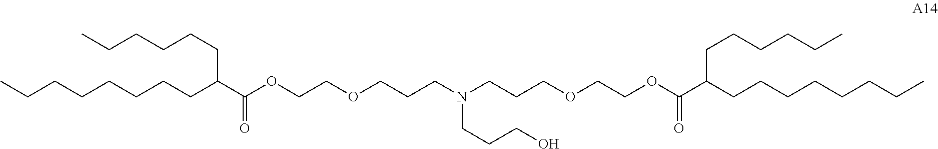 [SMILES: CCCCCCCCC(CCCCCC)C(=O)OCCOCCCN(CCCO)CCCOCCOC(=O)C(CCCCCC)CCCCCCCC]

Di(pentadecan-7-yl) 3,3′-((((3-hydroxypropyl)azanediyl)bis(propane-3,1-diyl))bis(oxy))dipropionate A15.

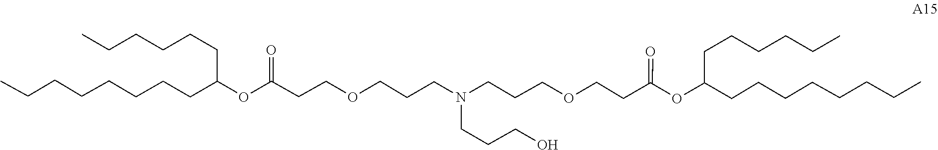 [SMILES: CCCCCCCCC(CCCCCC)OC(=O)CCOCCCN(CCCO)CCCOCCC(=O)OC(CCCCCC)CCCCCCCC]

Bis(1,1,1,15,15,15-hexafluoropentadecan-7-yl) 7,7′-((3-hydroxypropyl)-azanediyl)diheptanoate A16.

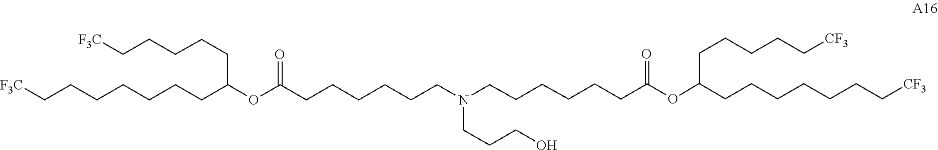 [SMILES: O=C(CCCCCCN(CCCO)CCCCCCC(=O)OC(CCCCCCCC(F)(F)F)CCCCCC(F)(F)F)OC(CCCCCCCC(F)(F)F)CCCCCC(F)(F)F]

1,1,1,15,15,15-Hexafluoropentadecan-7-yl 8-((3-hydroxypropyl)(6-oxo-6-((11,11,11-trifluoroundecyl)oxy)hexyl)amino)octanoate A17.

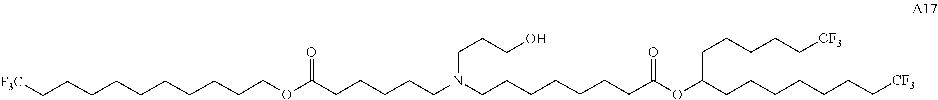 [SMILES: O=C(CCCCCN(CCCO)CCCCCCCC(=O)OC(CCCCCCCC(F)(F)F)CCCCCC(F)(F)F)OCCCCCCCCCCC(F)(F)F]

Pentadecan-7-yl 8-((3-hydroxypropyl)(6-oxo-6-((11,11,11-trifluoroundecyl)oxy)-hexyl)amino)octanoate A18.

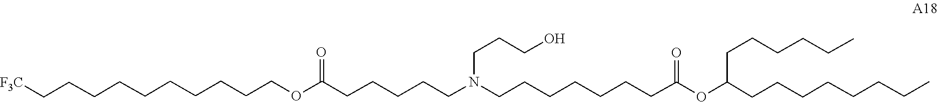 [SMILES: CCCCCCCCC(CCCCCC)OC(=O)CCCCCCCN(CCCO)CCCCCC(=O)OCCCCCCCCCCC(F)(F)F]

1,1,1,17,17,17-Hexafluoroheptadecan-9-yl 8-((3-hydroxypropyl)(6-oxo-6-((11,11,11-trifluoroundecyl)oxy)hexyl)amino)octanoate A19.

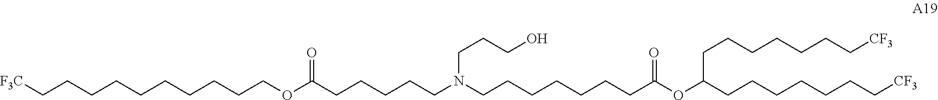 [SMILES: O=C(CCCCCN(CCCO)CCCCCCCC(=O)OC(CCCCCCCC(F)(F)F)CCCCCCCC(F)(F)F)OCCCCCCCCCCC(F)(F)F]

Heptadecan-9-yl 8-((3-hydroxypropyl)(6-oxo-6-((11,11,11-trifluoroundecyl)oxy)-hexyl)amino)octanoate A20.

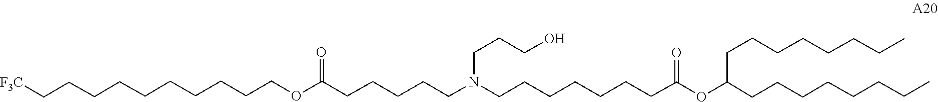 [SMILES: CCCCCCCCC(CCCCCCCC)OC(=O)CCCCCCCN(CCCO)CCCCCC(=O)OCCCCCCCCCCC(F)(F)F]

8,8′-((4-Hydroxybutyl)azanediyl)bis(N-hexyl-N-octyloctanamide) A21.

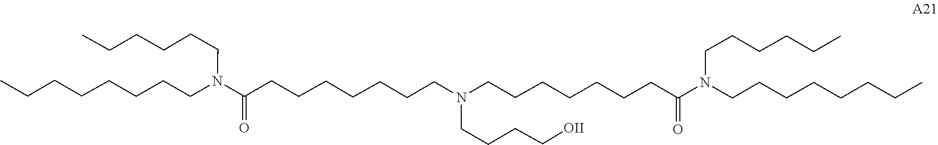

((4-Hydroxybutyl)azanediyl)bis(hexane-6,1-diyl) bis(hexyl(octyl)carbamate) A22.

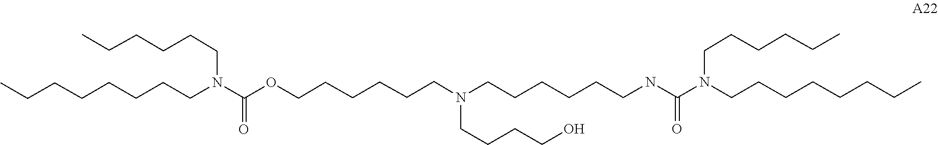 [SMILES: CCCCCCCCN(CCCCCC)C(=O)NCCCCCCN(CCCCO)CCCCCCOC(=O)N(CCCCCC)CCCCCCCC]

N-(4-Hydroxybutyl)-N,N-di(7-(pentadecan-7-oxycarbonyloxy)heptyl)amine A23.

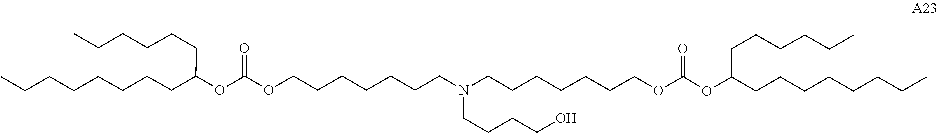 [SMILES: CCCCCCCCC(CCCCCC)OC(=O)OCCCCCCCN(CCCCO)CCCCCCCOC(=O)OC(CCCCCC)CCCCCCCC]

((((4-Hydroxybutyl)azanediyl)bis(propane-3,1-diyl))bis(oxy))bis(ethane-2,1-diyl) bis(2-hexyldecanoate) A24.

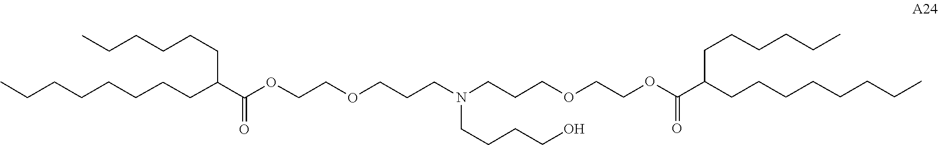 [SMILES: CCCCCCCCC(CCCCCC)C(=O)OCCOCCCN(CCCCO)CCCOCCOC(=O)C(CCCCCC)CCCCCCCC]

Di(pentadecan-7-yl) 3,3′-((((4-hydroxybutyl)azanediyl)bis(propane-3,1-diyl))bis(oxy))dipropionate A25.

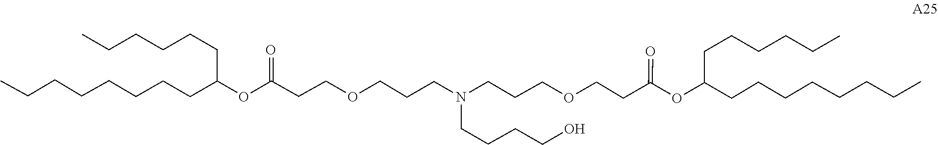 [SMILES: CCCCCCCCC(CCCCCC)OC(=O)CCOCCCN(CCCCO)CCCOCCC(=O)OC(CCCCCC)CCCCCCCC]

Bis(1,1,1,15,15,15-hexafluoropentadecan-7-yl) 7,7′-((4-hydroxybutyl)-azanediyl)diheptanoate A26.

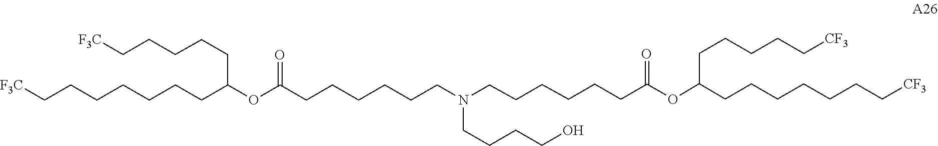 [SMILES: O=C(CCCCCCN(CCCCO)CCCCCCC(=O)OC(CCCCCCCC(F)(F)F)CCCCCC(F)(F)F)OC(CCCCCCCC(F)(F)F)CCCCCC(F)(F)F]

1,1,1,15,15,15-Hexafluoropentadecan-7-yl 8-((4-hydroxybutyl)(6-oxo-6-((11,11,11-trifluoroundecyl)oxy)hexyl)amino)octanoate A27.

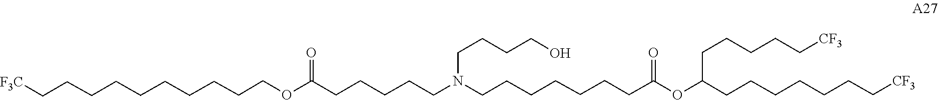 [SMILES: O=C(CCCCCN(CCCCO)CCCCCCCC(=O)OC(CCCCCCCC(F)(F)F)CCCCCC(F)(F)F)OCCCCCCCCCCC(F)(F)F]

Pentadecan-7-yl 8-((4-hydroxybutyl)(6-oxo-6-((11,11,11-trifluoroundecyl)oxy)-hexyl)amino)octanoate A28.

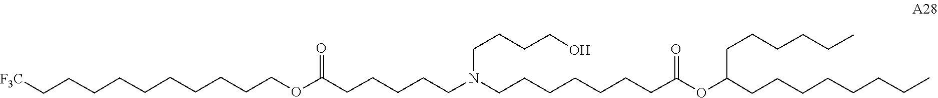 [SMILES: CCCCCCCCC(CCCCCC)OC(=O)CCCCCCCN(CCCCO)CCCCCC(=O)OCCCCCCCCCCC(F)(F)F]

1,1,1,17,17,17-Hexafluoroheptadecan-9-yl 8-((4-hydroxybutyl)(6-oxo-6-((11,11,11-trifluoroundecyl)oxy)hexyl)amino)octanoate A29.

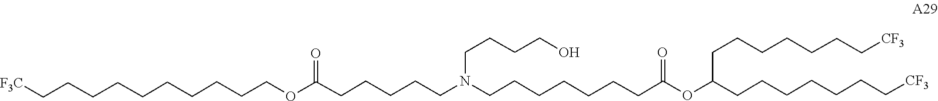 [SMILES: O=C(CCCCCN(CCCCO)CCCCCCCC(=O)OC(CCCCCCCC(F)(F)F)CCCCCCCC(F)(F)F)OCCCCCCCCCCC(F)(F)F]

Heptadecan-9-yl 8-((4-hydroxybutyl)(6-oxo-6-((11,11,11-trifluoroundecyl)oxy)-hexyl)amino)octanoate A30.

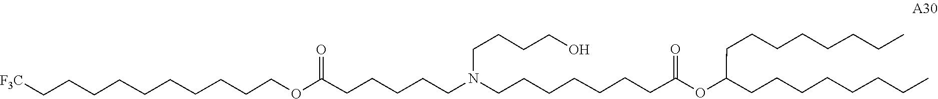 [SMILES: CCCCCCCCC(CCCCCCCC)OC(=O)CCCCCCCN(CCCCO)CCCCCC(=O)OCCCCCCCCCCC(F)(F)F]

8,8′-((5-Hydroxypentyl)azanediyl)bis(N-hexyl-N-octyloctanamide) A31.

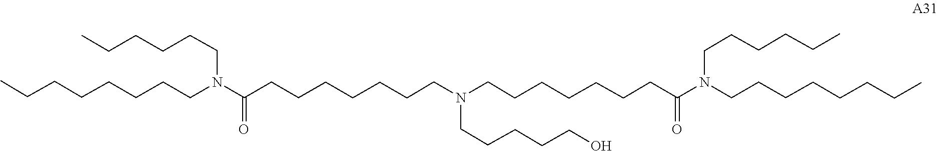 [SMILES: CCCCCCCCN(CCCCCC)C(=O)CCCCCCCN(CCCCCO)CCCCCCCC(=O)N(CCCCCC)CCCCCCCC]

((5-Hydroxypentyl)azanediyl)bis(hexane-6,1-diyl) bis(hexyl(octyl)carbamate) A32.

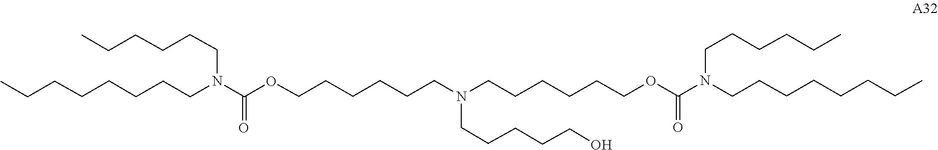 [SMILES: CCCCCCCCN(CCCCCC)C(=O)OCCCCCCN(CCCCCO)CCCCCCOC(=O)N(CCCCCC)CCCCCCCC]

N-(5-Hydroxypentyl)-N,N-di(7-(pentadecan-7-oxycarbonyloxy)heptyl)amine A33.

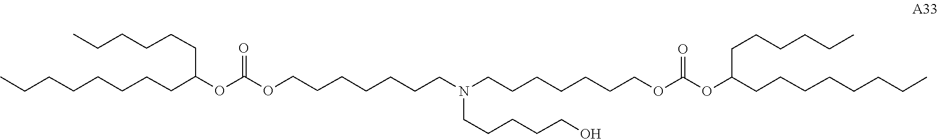 [SMILES: CCCCCCCCC(CCCCCC)OC(=O)OCCCCCCCN(CCCCCO)CCCCCCCOC(=O)OC(CCCCCC)CCCCCCCC]

((((5-Hydroxypentyl)azanediyl)bis(propane-3,1-diyl))bis(oxy))bis(ethane-2,1-diyl) bis(2-hexyldecanoate) A34.

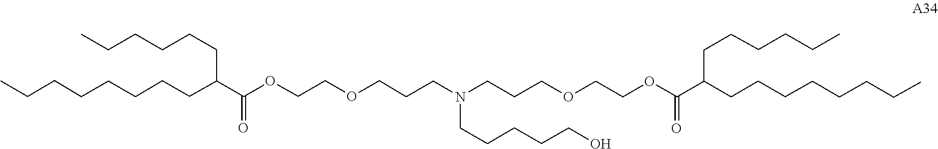 [SMILES: CCCCCCCCC(CCCCCC)C(=O)OCCOCCCN(CCCCCO)CCCOCCOC(=O)C(CCCCCC)CCCCCCCC]

Di(pentadecan-7-yl) 3,3′-((((5-hydroxypentyl)azanediyl)bis(propane-3,1-diyl))bis(oxy))dipropionate A35.

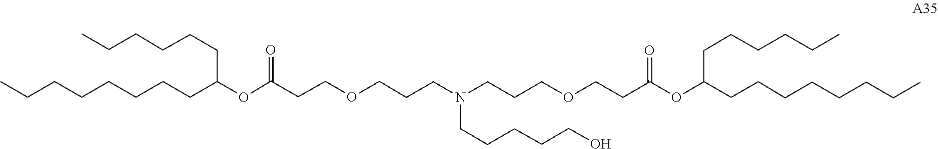 [SMILES: CCCCCCCCC(CCCCCC)OC(=O)CCOCCCN(CCCCCO)CCCOCCC(=O)OC(CCCCCC)CCCCCCCC]

Bis(1,1,1,15,15,15-hexafluoropentadecan-7-yl) 7,7′-((5-hydroxypentyl)-azanediyl)diheptanoate A36.

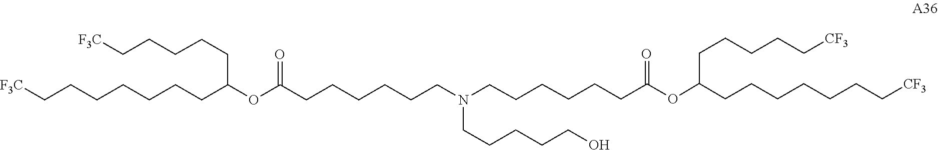 [SMILES: O=C(CCCCCCN(CCCCCO)CCCCCCC(=O)OC(CCCCCCCC(F)(F)F)CCCCCC(F)(F)F)OC(CCCCCCCC(F)(F)F)CCCCCC(F)(F)F]

1,1,1,15,15,15-Hexafluoropentadecan-7-yl 8-((5-hydroxypentyl)(6-oxo-6-((11,11,11-trifluoroundecyl)oxy)hexyl)amino)octanoate A37.

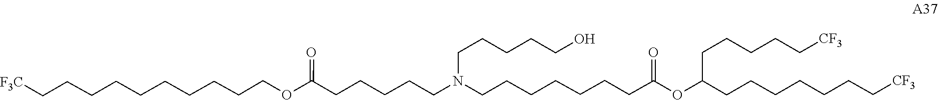 [SMILES: O=C(CCCCCN(CCCCCO)CCCCCCCC(=O)OC(CCCCCCCC(F)(F)F)CCCCCC(F)(F)F)OCCCCCCCCCCC(F)(F)F]

Pentadecan-7-yl 8-((5-hydroxypentyl)(6-oxo-6-((11,11,11-trifluoroundecyl)oxy)-hexyl)amino)octanoate A38.

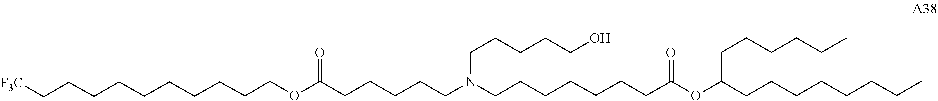 [SMILES: CCCCCCCCC(CCCCCC)OC(=O)CCCCCCCN(CCCCCO)CCCCCC(=O)OCCCCCCCCCCC(F)(F)F]

1,1,1,17,17,17-Hexafluoroheptadecan-9-yl 8-((5-hydroxypentyl)(6-oxo-6-((11,11,11-trifluoroundecyl)oxy)hexyl)amino)octanoate A39.

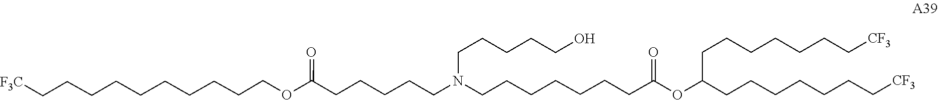 [SMILES: O=C(CCCCCN(CCCCCO)CCCCCCCC(=O)OC(CCCCCCCC(F)(F)F)CCCCCCCC(F)(F)F)OCCCCCCCCCCC(F)(F)F]

Heptadecan-9-yl 8-((5-hydroxypentyl)(6-oxo-6-((11,11,11-trifluoroundecyl)oxy)-hexyl)amino)octanoate A40.

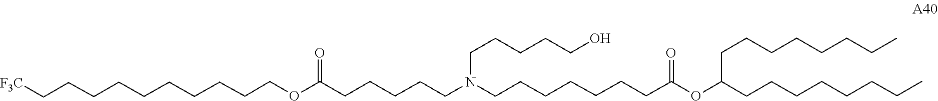 [SMILES: CCCCCCCCC(CCCCCCCC)OC(=O)CCCCCCCN(CCCCCO)CCCCCC(=O)OCCCCCCCCCCC(F)(F)F]

N-(2-Hydroxyethyl)-N,N-di(5-(pentadecan-7-oxycarbonyloxy)pentyl)amine A41.

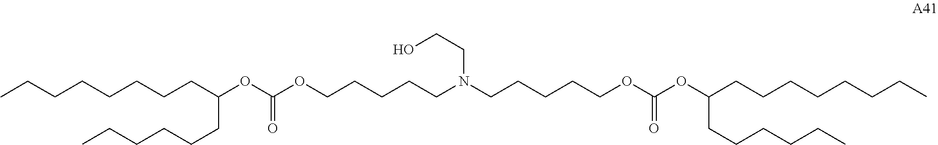 [SMILES: CCCCCCCCC(CCCCCC)OC(=O)OCCCCCN(CCO)CCCCCOC(=O)OC(CCCCCC)CCCCCCCC]

N-(2-Hydroxyethyl)-N,N-di(7-(heptadecan-9-oxycarbonyloxy)heptyl)amine A42.

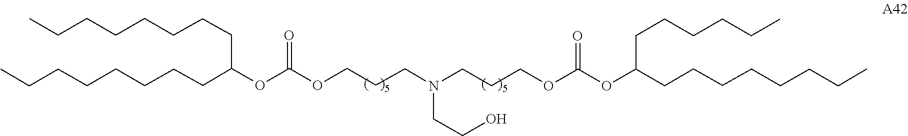

((((2-Hydroxyethyl)azanediyl)bis(propane-3,1-diyl))bis(oxy))bis(ethane-2,1-diyl) bis(2-hexyldecanoate) A43.

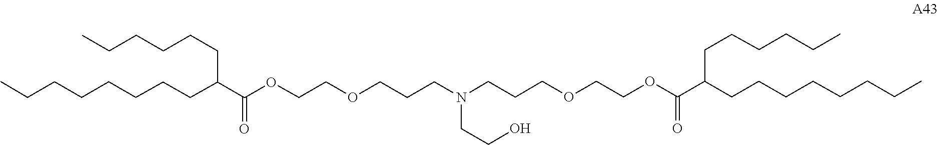 [SMILES: CCCCCCCCC(CCCCCC)C(=O)OCCOCCCN(CCO)CCCOCCOC(=O)C(CCCCCC)CCCCCCCC]

Di(pentadecan-7-yl) 3,3′-((((2-hydroxyethyl)azanediyl)bis(propane-3,1-diyl))bis(oxy))dipropionate A44.

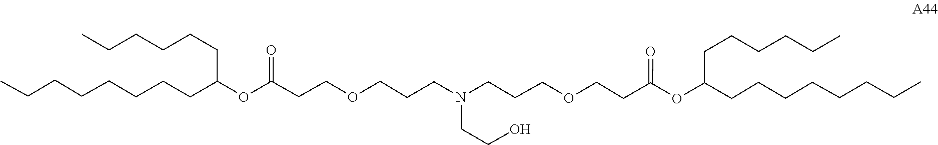 [SMILES: CCCCCCCCC(CCCCCC)OC(=O)CCOCCCN(CCO)CCCOCCC(=O)OC(CCCCCC)CCCCCCCC]

Di(pentadecan-7-yl) 10-(2-hydroxyethyl)-4,7,13,16-tetraoxa-10-azanonadecane-dioate A45.

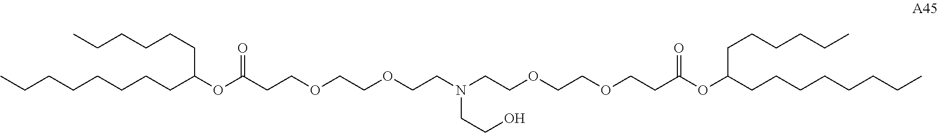 [SMILES: CCCCCCCCC(CCCCCC)OC(=O)CCOCCOCCN(CCO)CCOCCOCCC(=O)OC(CCCCCC)CCCCCCCC]

Heptadecan-9-yl (6-((4-hydroxybutyl)(6-(((undecyloxy)carbonyl)amino)hexyl)-amino)hexyl)carbamate A47.

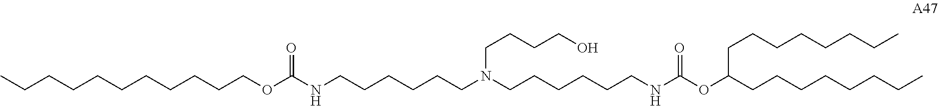 [SMILES: CCCCCCCCCCCOC(=O)NCCCCCCN(CCCCO)CCCCCCNC(=O)OC(CCCCCCCC)CCCCCCCC]

((((2-Hydroxyethyl)azanediyl)bis(ethane-2,1-diyl))bis(oxy))bis(ethane-2,1-diyl) bis(2-hexyldecanoate) A50.

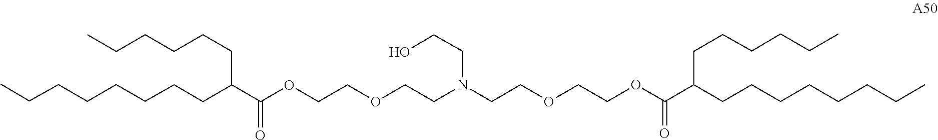 [SMILES: CCCCCCCCC(CCCCCC)C(=O)OCCOCCN(CCO)CCOCCOC(=O)C(CCCCCC)CCCCCCCC]

Di(heptadecan-9-yl) 10-(2-hydroxyethyl)-4,7,13,16-tetraoxa-10-azanonadecane-dioate A51.

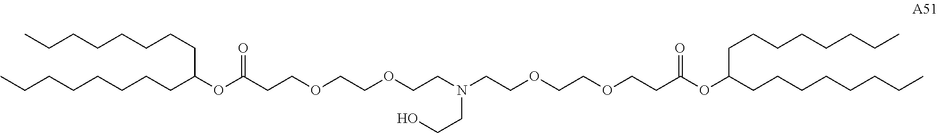 [SMILES: CCCCCCCCC(CCCCCCCC)OC(=O)CCOCCOCCN(CCO)CCOCCOCCC(=O)OC(CCCCCCCC)CCCCCCCC]

8,8′-((2-Hydroxyethyl)azanediyl)bis(N-hexyl-N-octyloctanamide) A52.

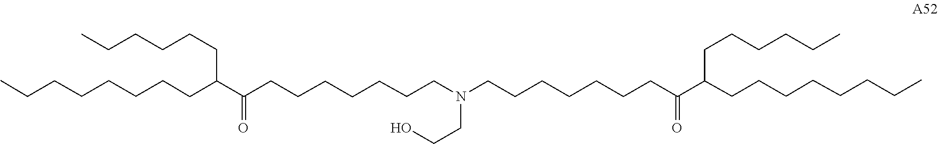 [SMILES: CCCCCCCCC(CCCCCC)C(=O)CCCCCCCN(CCO)CCCCCCCC(=O)C(CCCCCC)CCCCCCCC]

Heptadecan-9-yl 8-((2-hydroxyethyl)(3-oxo-4,7,10,13-tetraoxatetracosyl)amino)-octanoate A53.

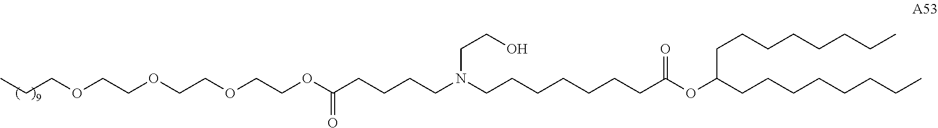

Heptadecan-9-yl 8-((2-hydroxyethyl)(6-oxo-6-(2-(2-(pentyloxy)ethoxy)ethoxy)-hexyl)amino)octanoate A54.

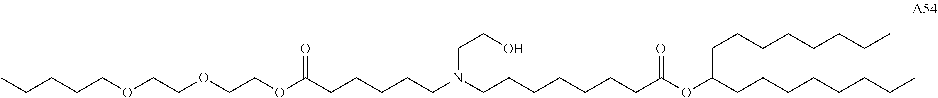 [SMILES: CCCCCCCCC(CCCCCCCC)OC(=O)CCCCCCCN(CCO)CCCCCC(=O)OCCOCCOCCCCC]

Di(heptadecan-9-yl) 8,8′-((4-hydroxybutyl)azanediyl)dioctanoate B42.

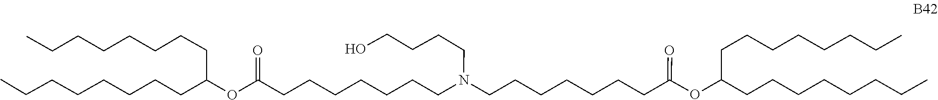 [SMILES: CCCCCCCCC(CCCCCCCC)OC(=O)CCCCCCCN(CCCCO)CCCCCCCC(=O)OC(CCCCCCCC)CCCCCCCC]

Di(heptadecan-9-yl) 8,8′-((3-(dimethylamino)propyl)azanediyl)dioctanoate B43.

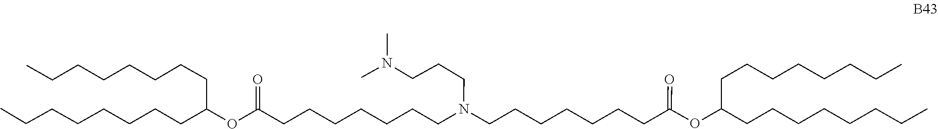 [SMILES: CCCCCCCCC(CCCCCCCC)OC(=O)CCCCCCCN(CCCCCCCC(=O)OC(CCCCCCCC)CCCCCCCC)CCCN(C)C]

Di(heptadecan-9-yl) 8,8′-((2-hydroxyethyl)azanediyl)dioctanoate B45.

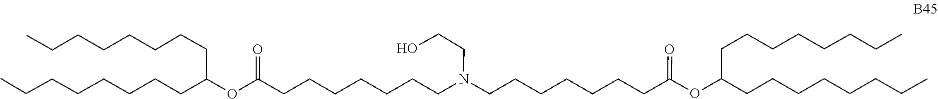 [SMILES: CCCCCCCCC(CCCCCCCC)OC(=O)CCCCCCCN(CCO)CCCCCCCC(=O)OC(CCCCCCCC)CCCCCCCC]

5-((2-Hydroxyethyl)(7-((2-octyldecanoyl)oxy)heptyl)amino)pentyl dodecanoate B48.

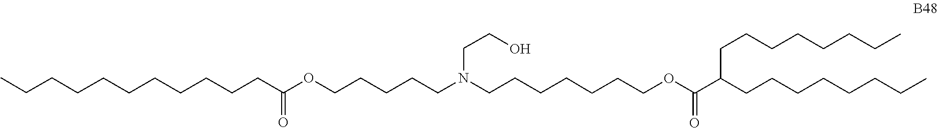 [SMILES: CCCCCCCCCCCC(=O)OCCCCCN(CCO)CCCCCCCOC(=O)C(CCCCCCCC)CCCCCCCC]

The examples set forth above are provided to give those of ordinary skill in the art with a complete disclosure and description of how to make and use the claimed embodiments and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

What is claimed is:

1. A compound of Formula (I): 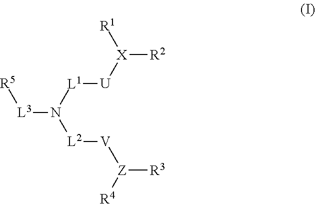
or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, or hydrate thereof; wherein:
R¹ is hydrogen, C₁₋₂₀ alkyl, C₁₋₂₀ heteroalkyl, C₂₋₂₀ alkenyl, or C₂₋₂₀ alkynyl;
R², R³, and R⁴ are each independently C₁₋₂₀ alkyl, C₁₋₂₀ heteroalkyl, C₂₋₂₀ alkenyl, or C₂₋₂₀ alkynyl;
R⁵ is —C(O)OR^{5a}, —OR^{a}, or —OC(O)R^{5a}; wherein each R^{5a} is independently hydrogen, C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl;
L¹, L², and L³ are each independently C₁₋₂₀ alkylene, C₁₋₂₀ heteroalkylene, C₂₋₂₀ alkenylene, or C₂₋₂₀ alkynylene;
U and V are each independently —C(O)—, —C(O)O—, or —OC(O)O—; and
X and Y are each independently C(H) or N;
with the proviso that, when U and V are each —C(O)O—, and X and Z are each C(H), then at least one of R¹, R², R³, and R⁴ is substituted C₁₋₂₀ alkyl, or at least one of L¹ and L² is C₁₋₂₀ heteroalkylene;
wherein each alkyl, alkylene, heteroalkyl, heteroalkylene, alkenyl, alkenylene, alkynyl, alkynylene, cycloalkyl, aryl, aralkyl, heteroaryl, and heterocyclyl is optionally substituted with one or more, in one embodiment, one, two, three, or four, substituents Q, wherein each Q is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{b})OR^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, nitro, imino, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OP(O)(OR^{f})OR^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{e}, —NR^{e}C(O)N^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

2. The compound of claim 1, wherein X is N.

3. The compound of claim 1, wherein X is C(H).

4. The compound of any one of claims 1 to 3, wherein Z is N.

5. The compound of any one of claims 1 to 3, wherein Z is C(H).

6. The compound of claim 1, having the structure of Formula (II): 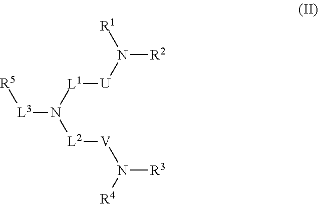
or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, or hydrate thereof.

7. The compound of any one of claims 1 to 6, wherein U is —C(O)—.

8. The compound of any one of claims 1 to 6, wherein U is —C(O)O—.

9. The compound of any one of claims 1 to 8, wherein V is —C(O)—.

10. The compound of any one of claims 1 to 8, wherein V is —C(O)O—.

11. The compound of claim 1 or 6, having the structure of Formula (III): 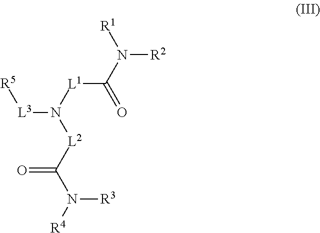
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof.

12. The compound of claim 1 or 6, having the structure of Formula (IV): 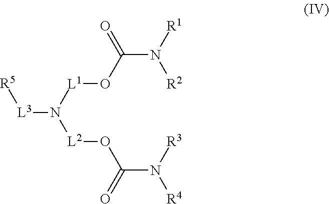
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof.

13. The compound of claim 1, having the structure of Formula (V): 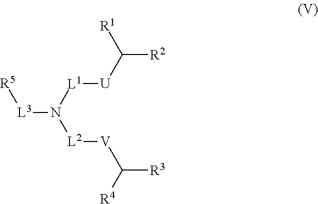
or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, or hydrate thereof.

14. The compound of any one of claims 1 to 5 and 13, wherein U is —C(O)—.

15. The compound of any one of claims 1 to 5 and 13, wherein U is —C(O)O—.

16. The compound of any one of claims 1 to 5 and 13, wherein U is —OC(O)O—.

17. The compound of any one of claims 1 to 5 and 13 to 16, wherein V is —C(O)—.

18. The compound of any one of claims 1 to 5 and 13 to 16, wherein V is —C(O)O—.

19. The compound of any one of claims 1 to 5 and 13 to 16, wherein V is —OC(O)O—.

20. The compound of claim 1 or 13, having the structure of Formula (VI): 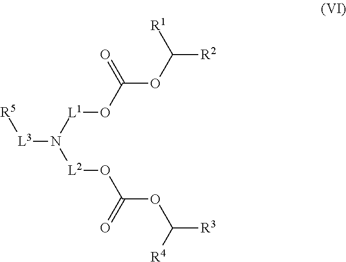
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof.

21. The compound of claim 1 or 13, having the structure of Formula (VII): 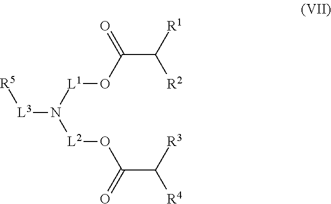
or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, or hydrate thereof.

22. The compound of claim 1 or 13, having the structure of Formula (VII): 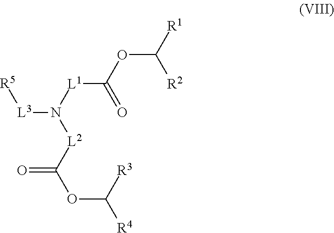
or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, or hydrate thereof.

23. The compound of any one of claims 1 to 22, wherein L¹ is C₁₋₂₀ alkylene, C₁₋₂₀ heteroalkylene, or C₂₋₂₀ alkenylene, each optionally substituted with one or more substituents Q.

24. The compound of any one of claims 1 to 23, wherein L¹ is C₁₋₁₀ alkylene, optionally substituted with one or more substituents Q.

25. The compound of any one of claims 1 to 24, wherein L¹ is ethanediyl, propanediyl, butanediyl, pentanediyl, hexanediyl, heptanediyl, octanediyl, nonanediyl, or decanediyl, each optionally substituted with one or more substituents Q.

26. The compound of any one of claims 1 to 24, wherein L¹ is unsubstituted C₁₋₁₀ alkyl.

27. The compound of any one of claims 1 to 26, wherein L¹ is pentane-1,5-diyl, hexane-1,6-diyl, or heptane-1,7-diyl.

28. The compound of any one of claims 1 to 23, wherein L¹ is C₁₋₁₀ heteroalkylene, optionally substituted with one or more substituents Q.

29. The compound of any one of claims 1 to 23 and 28, wherein L¹ is unsubstituted C₁₋₁₀ heteroalkylene.

30. The compound of any one of claims 1 to 23 and 28, wherein L¹ is —N(H)—C₁₋₂₀ alkenylene or —N(H)—C₁₋₂₀ heteroalkenylene, each optionally substituted with one or more substituents Q.

31. The compound of any one of claims 1 to 23, 28, and 30, wherein L¹ is —N(H)—C₁₋₂₀ alkenylene, optionally substituted with one or more substituents Q.

32. The compound of any one of claims 1 to 23, 28, and 30, wherein L¹ is —N(H)—C₁₋₁₀ alkenylene, optionally substituted with one or more substituents Q.

33. The compound of any one of claims 1 to 23, 28, and 29, wherein L¹ is —(CH₂)₂O(CH₂)₂—, —(CH₂)₂O(CH₂)₃—, —(CH₂)₂O(CH₂)₂O(CH₂)₂—, or —N(H)(CH₂)₆—.

34. The compound of any one of claims 1 to 33, wherein L² is C₁₋₂₀ alkylene, C₁₋₂₀ heteroalkylene, or C₂₋₂₀ alkenylene, each optionally substituted with one or more substituents Q.

35. The compound of any one of claims 1 to 34, wherein L² is C₁₋₁₀ alkylene, optionally substituted with one or more substituents Q.

36. The compound of any one of claims 1 to 35, wherein L² is ethanediyl, propanediyl, butanediyl, pentanediyl, hexanediyl, heptanediyl, octanediyl, nonanediyl, or decanediyl, each optionally substituted with one or more substituents Q.

37. The compound of any one of claims 1 to 35, wherein L² is unsubstituted C₁₋₁₀ alkyl.

38. The compound of any one of claims 1 to 37, wherein L² is pentane-1,5-diyl, hexane-1,6-diyl, or heptane-1,7-diyl.

39. The compound of any one of claims 1 to 34, wherein L² is C₁₋₁₀ heteroalkylene, optionally substituted with one or more substituents Q.

40. The compound of any one of claims 1 to 34 and 39, wherein L² is unsubstituted C₁₋₂₀ heteroalkylene.

41. The compound of any one of claims 1 to 34 and 39, wherein L¹ is —N(H)—C₁₋₂₀ alkenylene or —N(H)—C₁₋₂₀ heteroalkenylene, each optionally substituted with one or more substituents Q.

42. The compound of any one of claims 1 to 34, 39, and 41, wherein L¹ is —N(H)—C₁₋₂₀ alkenylene, optionally substituted with one or more substituents Q.

43. The compound of any one of claims 1 to 34, 39, and 41, wherein L¹ is —N(H)—C₁₋₁₀ alkenylene, optionally substituted with one or more substituents Q.

44. The compound of any one of claims 1 to 34, 39, and 40, wherein L² is —(CH₂)₂O(CH₂)₂—, —(CH₂)₂O(CH₂)₃—, —(CH₂)₂O(CH₂)₂O(CH₂)₂—, or —N(H)(CH₂)₆—.

45. The compound of claim 1, having the structure of Formula (IX): 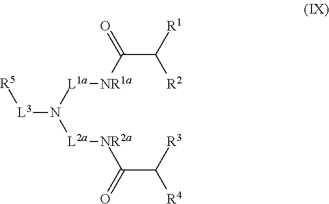
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof; wherein:
L^{1a} and L^{2a} are each independently C₁₋₂₀ alkylene or C₁₋₂₀ heteroalkylene, each optionally substituted with one or more substituents Q; and
R^{1a} and R^{2a} are each independently (i) hydrogen; or (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each optionally substituted with one or more substituents Q.

46. The compound of claim 1, having the structure of Formula (X): 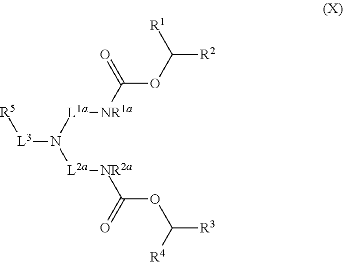
or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, or hydrate thereof; wherein:
L^{1a} and L^{2a} are each independently C₁₋₂₀ alkylene or C₁₋₂₀ heteroalkylene, each optionally substituted with one or more substituents Q; and
R^{1a} and R^{2a} are each independently (i) hydrogen; or (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each optionally substituted with one or more substituents Q.

47. The compound of claim 45 or 46, wherein R^{1a} is hydrogen.

48. The compound of any one of claims 45 to 47, wherein R^{2a} is hydrogen.

49. The compound of any one of claims 45 to 48, wherein L^{1a} is C₁₋₂₀ alkylene, C₁₋₂₀ heteroalkylene, or C₂₋₂₀ alkenylene, each optionally substituted with one or more substituents Q.

50. The compound of any one of claims 45 to 49, wherein L^{1a} is C₁₋₁₀ alkylene, optionally substituted with one or more substituents Q.

51. The compound of any one of claims 45 to 50, wherein L^{1a} is ethanediyl, propanediyl, butanediyl, pentanediyl, hexanediyl, heptanediyl, octanediyl, nonanediyl, or decanediyl, each optionally substituted with one or more substituents Q.

52. The compound of any one of claims 45 to 50, wherein L^{1a} is unsubstituted C₁₋₁₀ alkyl.

53. The compound of any one of claims 45 to 52, wherein L^{1a} is pentane-1,5-diyl, hexane-1,6-diyl, or heptane-1,7-diyl.

54. The compound of any one of claims 45 to 49, wherein L^{1a} is C₁₋₁₀ heteroalkylene, optionally substituted with one or more substituents Q.

55. The compound of any one of claims 45 to 54, wherein L^{2a} is C₁₋₂₀ alkylene, C₁₋₂₀ heteroalkylene, or C₂₋₂₀ alkenylene, each optionally substituted with one or more substituents Q.

56. The compound of any one of claims 45 to 55, wherein L^{2a} is C₁₋₁₀ alkylene, optionally substituted with one or more substituents Q.

57. The compound of any one of claims 45 to 56, wherein L^{2a} is ethanediyl, propanediyl, butanediyl, pentanediyl, hexanediyl, heptanediyl, octanediyl, nonanediyl, or decanediyl, each optionally substituted with one or more substituents Q.

58. The compound of any one of claims 45 to 56, wherein L^{2a} is unsubstituted C₁₋₁₀ alkyl.

59. The compound of any one of claims 45 to 58, wherein L^{2a} is pentane-1,5-diyl, hexane-1,6-diyl, or heptane-1,7-diyl.

60. The compound of any one of claims 45 to 55, wherein L^{2a} is C₁₋₁₀ heteroalkylene, optionally substituted with one or more substituents Q.

61. The compound of any one of claims 1 to 60, wherein R¹ is hydrogen, C₁₋₂₀ alkyl, C₁₋₂₀ heteroalkyl, or C₂₋₂₀ alkenyl, each optionally substituted with one or more substituents Q.

62. The compound of any one of claims 1 to 61, wherein R¹ is C₁₋₁₀ alkyl, optionally substituted with one or more substituents Q.

63. The compound of any one of claims 1 to 62, wherein R¹ is C₁₋₁₀ alkyl, substituted with one, two, or three fluoro.

64. The compound of any one of claims 1 to 63, wherein R¹ is trifluoromethyl, trifluoroethyl, trifluoropropyl, trifluorobutyl, trifluoropentyl, trifluorohexyl, trifluoroheptyl, trifluorooctyl, trifluorononyl, or trifluorodecyl.

65. The compound of any one of claims 1 to 64, wherein R¹ is 6,6,6-trifluorohex-1-yl, 8,8,8-trifluorooct-1-yl, or 10,10,10-trifluorodec-1-yl.

66. The compound of any one of claims 1 to 62, wherein R¹ is unsubstituted C₁₋₁₀ alkyl.

67. The compound of any one of claims 1 to 62, wherein R¹ is methyl, ethyl, propyl, butyl, pentyl, hexyl, heptyl, octyl, nonyl, or decyl, each optionally substituted with one or more substituents Q.

68. The compound of any one of claims 1 to 62 and 67, wherein R¹ is n-hexyl, or n-octyl.

69. The compound of any one of claims 1 to 61, wherein R¹ is hydrogen.

70. The compound of any one of claims 1 to 69, wherein R² is C₁₋₂₀ alkyl, C₁₋₂₀ heteroalkyl, or C₂₋₂₀ alkenyl, each optionally substituted with one or more substituents Q.

71. The compound of any one of claims 1 to 70, wherein R² is C₁₋₁₀ alkyl, optionally substituted with one or more substituents Q.

72. The compound of any one of claims 1 to 71, wherein R² is C₁₋₁₀ alkyl, substituted with one, two, or three fluoro.

73. The compound of any one of claims 1 to 72, wherein R² is trifluoromethyl, trifluoroethyl, trifluoropropyl, trifluorobutyl, trifluoropentyl, trifluorohexyl, trifluoroheptyl, trifluorooctyl, trifluorononyl, or trifluorodecyl.

74. The compound of any one of claims 1 to 73, wherein R² is 6,6,6-trifluorohex-1-yl, 8,8,8-trifluorooct-1-yl, or 10,10,10-trifluorodec-1-yl.

75. The compound of any one of claims 1 to 71, wherein R² is unsubstituted C₁₋₁₀ alkyl.

76. The compound of any one of claims 1 to 71, wherein R² is methyl, ethyl, propyl, butyl, pentyl, hexyl, heptyl, octyl, nonyl, or decyl, each optionally substituted with one or more substituents Q.

77. The compound of any one of claims 1 to 71 and 76, wherein R² is n-hexyl or n-octyl.

78. The compound of any one of claims 1 to 77, wherein R³ is C₁₋₂₀ alkyl, C₁₋₂₀ heteroalkyl, or C₂₋₂₀ alkenyl, each optionally substituted with one or more substituents Q.

79. The compound of any one of claims 1 to 78, wherein R³ is C₁₋₁₀ alkyl, optionally substituted with one or more substituents Q.

80. The compound of any one of claims 1 to 79, wherein R³ is C₁₋₁₀ alkyl, substituted with one, two, or three fluoro.

81. The compound of any one of claims 1 to 80, wherein R³ is trifluoromethyl, trifluoroethyl, trifluoropropyl, trifluorobutyl, trifluoropentyl, trifluorohexyl, trifluoroheptyl, trifluorooctyl, trifluorononyl, or trifluorodecyl.

82. The compound of any one of claims 1 to 81, wherein R³ is 6,6,6-trifluorohex-1-yl, 8,8,8-trifluorooct-1-yl, or 10,10,10-trifluorodec-1-yl.

83. The compound of any one of claims 1 to 79, wherein R³ is unsubstituted C₁₋₁₀ alkyl.

84. The compound of any one of claims 1 to 79, wherein R³ is methyl, ethyl, propyl, butyl, pentyl, hexyl, heptyl, octyl, nonyl, or decyl, each optionally substituted with one or more substituents Q.

85. The compound of any one of claims 1 to 79 and 84, wherein R³ is n-hexyl or n-octyl.

86. The compound of any one of claims 1 to 85, wherein R⁴ is C₁₋₂₀ alkyl, C₁₋₂₀ heteroalkyl, or C₂₋₂₀ alkenyl, each optionally substituted with one or more substituents Q.

87. The compound of any one of claims 1 to 86, wherein R⁴ is C₁₋₁₀ alkyl, optionally substituted with one or more substituents Q.

88. The compound of any one of claims 1 to 87, wherein R³ is C₁₋₁₀ alkyl, substituted with one, two, or three fluoro.

89. The compound of any one of claims 1 to 88, wherein R⁴ is trifluoromethyl, trifluoroethyl, trifluoropropyl, trifluorobutyl, trifluoropentyl, trifluorohexyl, trifluoroheptyl, trifluorooctyl, trifluorononyl, or trifluorodecyl.

90. The compound of any one of claims 1 to 89, wherein R⁴ is 6,6,6-trifluorohex-1-yl, 8,8,8-trifluorooct-1-yl, or 10,10,10-trifluorodec-1-yl.

91. The compound of any one of claims 1 to 87, wherein R⁴ is unsubstituted C₁₋₁₀ alkyl.

92. The compound of any one of claims 1 to 87, wherein R⁴ is methyl, ethyl, propyl, butyl, pentyl, hexyl, heptyl, octyl, nonyl, or decyl, each optionally substituted with one or more substituents Q.

93. The compound of any one of claims 1 to 87 and 92, wherein R⁴ is n-hexyl or n-octyl.

94. The compound of any one of claims 1 to 93, wherein R⁵ is —OR^{5a}.

95. The compound of any one of claims 1 to 94, wherein R⁵ is C₁₋₆ alkoxy, optionally substituted with one or more substituents Q.

96. The compound of any one of claims 1 to 94, wherein R⁵ is hydroxyl or methoxy.

97. The compound of any one of claims 1 to 94, wherein R⁵ is hydroxyl.

98. The compound of any one of claims 1 to 97, wherein L³ is C₁₋₂₀ alkylene, C₁₋₂₀ heteroalkylene, or C₂₋₂₀ alkenylene, each optionally substituted with one or more substituents Q.

99. The compound of any one of claims 1 to 98, wherein L³ is C₁₋₁₀ alkylene, optionally substituted with one or more substituents Q.

100. The compound of any one of claims 1 to 99, wherein L³ is ethanediyl, propanediyl, butanediyl, pentanediyl, or hexanediyl, each optionally substituted with one or more substituents Q.

101. The compound of any one of claims 1 to 99, wherein L³ is unsubstituted C₁₋₁₀ alkyl.

102. The compound of any one of claims 1 to 101, wherein L³ is ethane-1,2-diyl, propane-1,3-diyl, butane-1,4-diyl, or pentane-1,5-diyl.

103. The compound of any one of claims 1 to 102, wherein L³ is ethane-1,2-diyl or butane-1,4-diyl.

104. A compound of:
8,8′-((2-hydroxyethyl)azanediyl)bis(N-hexyl-N-octyloctanamide) A1;
((2-hydroxyethyl)azanediyl)bis(hexane-6,1-diyl) bis(hexyl(octyl)carbamate) A2;
N-(2-hydroxyethyl)-N,N-di(7-(pentadecan-7-oxycarbonyloxy)heptyl)amine A3;
((((2-hydroxyethyl)azanediyl)bis(ethane-2,1-diyl))bis(oxy))bis(ethane-2,1-diyl) bis(2-hexyldecanoate) A4;
di(heptadecan-9-yl) 10-(2-hydroxyethyl)-4,7,13,16-tetraoxa-10-azanonadecanedioate A5;
bis(1,1,1,15,15,15-hexafluoropentadecan-7-yl) 7,7′-((2-hydroxyethyl)-azanediyl)diheptanoate A6;
1,1,1,15,15,15-hexafluoropentadecan-7-yl 8-((2-hydroxyethyl)(6-oxo-6-((11,11,11-trifluoroundecyl)oxy)hexyl)amino)octanoate A7;
pentadecan-7-yl 8-((2-hydroxyethyl)(6-oxo-6-((11,11,11-trifluoroundecyl)oxy)-hexyl)amino)octanoate A8;
1,1,1,17,17,17-hexafluoroheptadecan-9-yl 8-((2-hydroxyethyl)(6-oxo-6-((11,11,11-trifluoroundecyl)oxy)hexyl)amino)octanoate A9;
heptadecan-9-yl 8-((2-hydroxyethyl)(6-oxo-6-((11,11,11-trifluoroundecyl)oxy)-hexyl)amino)octanoate A10;
8,8′-((3-hydroxypropyl)azanediyl)bis(N-hexyl-N-octyloctanamide) A11;
((3-hydroxypropyl)azanediyl)bis(hexane-6,1-diyl) bis(hexyl(octyl)carbamate) A12;
N-(3-hydroxypropyl)-N,N-di(7-(pentadecan-7-oxycarbonyloxy)heptyl)amine A13;
((((3-hydroxypropyl)azanediyl)bis(propane-3,1-diyl))bis(oxy))bis(ethane-2,1-diyl) bis(2-hexyldecanoate) A14;
di(pentadecan-7-yl) 3,3′-((((3-hydroxypropyl)azanediyl)bis(propane-3,1-diyl))bis(oxy))dipropionate A15;
bis(1,1,1,15,15,15-hexafluoropentadecan-7-yl) 7,7′-((3-hydroxypropyl)-azanediyl)diheptanoate A16;
1,1,1,15,15,15-hexafluoropentadecan-7-yl 8-((3-hydroxypropyl)(6-oxo-6-((11,11,11-trifluoroundecyl)oxy)hexyl)amino)octanoate A17;
pentadecan-7-yl 8-((3-hydroxypropyl)(6-oxo-6-((11,11,11-trifluoroundecyl)oxy)-hexyl)amino)octanoate A18;
1,1,1,17,17,17-hexafluoroheptadecan-9-yl 8-((3-hydroxypropyl)(6-oxo-6-((11,11,11-trifluoroundecyl)oxy)hexyl)amino)octanoate A19;
heptadecan-9-yl 8-((3-hydroxypropyl)(6-oxo-6-((11,11,11-trifluoroundecyl)oxy)-hexyl)amino)octanoate A20;
8,8′-((4-hydroxybutyl)azanediyl)bis(N-hexyl-N-octyloctanamide) A21;
((4-hydroxybutyl)azanediyl)bis(hexane-6,1-diyl) bis(hexyl(octyl)carbamate) A22;
N-(4-hydroxybutyl)-N,N-di(7-(pentadecan-7-oxycarbonyloxy)heptyl)amine A23;
((((4-hydroxybutyl)azanediyl)bis(propane-3,1-diyl))bis(oxy))bis(ethane-2,1-diyl) bis(2-hexyldecanoate) A24;
di(pentadecan-7-yl) 3,3′-((((4-hydroxybutyl)azanediyl)bis(propane-3,1-diyl))bis(oxy))dipropionate A25;
bis(1,1,1,15,15,15-hexafluoropentadecan-7-yl) 7,7′-((4-hydroxybutyl)-azanediyl)diheptanoate A26;
1,1,1,15,15,15-hexafluoropentadecan-7-yl 8-((4-hydroxybutyl)(6-oxo-6-((11,11,11-trifluoroundecyl)oxy)hexyl)amino)octanoate A27;
pentadecan-7-yl 8-((4-hydroxybutyl)(6-oxo-6-((11,11,11-trifluoroundecyl)oxy)-hexyl)amino)octanoate A28;
1,1,1,17,17,17-hexafluoroheptadecan-9-yl 8-((4-hydroxybutyl)(6-oxo-6-((11,11,11-trifluoroundecyl)oxy)hexyl)amino)octanoate A29;
heptadecan-9-yl 8-((4-hydroxybutyl)(6-oxo-6-((11,11,11-trifluoroundecyl)oxy)-hexyl)amino)octanoate A30;
8,8′-((5-hydroxypentyl)azanediyl)bis(N-hexyl-N-octyloctanamide) A31;
((5-hydroxypentyl)azanediyl)bis(hexane-6,1-diyl) bis(hexyl(octyl)carbamate) A32;
N-(5-hydroxypentyl)-N,N-di(7-(pentadecan-7-oxycarbonyloxy)heptyl)amine A33;
((((5-hydroxypentyl)azanediyl)bis(propane-3,1-diyl))bis(oxy))bis(ethane-2,1-diyl) bis(2-hexyldecanoate) A34;
di(pentadecan-7-yl) 3,3′-((((5-hydroxypentyl)azanediyl)bis(propane-3,1-diyl))bis(oxy))dipropionate A35;
bis(1,1,1,15,15,15-hexafluoropentadecan-7-yl) 7,7′-((5-hydroxypentyl)-azanediyl)diheptanoate A36;
1,1,1,15,15,15-hexafluoropentadecan-7-yl 8-((5-hydroxypentyl)(6-oxo-6-((11,11,11-trifluoroundecyl)oxy)hexyl)amino)octanoate A37;
pentadecan-7-yl 8-((5-hydroxypentyl)(6-oxo-6-((11,11,11-trifluoroundecyl)oxy)-hexyl)amino)octanoate A38;
1,1,1,17,17,17-hexafluoroheptadecan-9-yl 8-((5-hydroxypentyl)(6-oxo-6-((11,11,11-trifluoroundecyl)oxy)hexyl)amino)octanoate A39;
heptadecan-9-yl 8-((5-hydroxypentyl)(6-oxo-6-((11,11,11-trifluoroundecyl)oxy)-hexyl)amino)octanoate A40;
N-(2-hydroxyethyl)-N,N-di(5-(pentadecan-7-oxycarbonyloxy)pentyl)amine A41;
N-(2-hydroxyethyl)-N,N-di(7-(heptadecan-9-oxycarbonyloxy)heptyl)amine A42;
((((2-hydroxyethyl)azanediyl)bis(propane-3,1-diyl))bis(oxy))bis(ethane-2,1-diyl) bis(2-hexyldecanoate) A43;
di(pentadecan-7-yl) 3,3′-((((2-hydroxyethyl)azanediyl)bis(propane-3,1-diyl))bis(oxy))dipropionate A44;
di(pentadecan-7-yl) 10-(2-hydroxyethyl)-4,7,13,16-tetraoxa-10-azanonadecane-dioate A45;
heptadecan-9-yl (6-((3-hydroxypropyl)(6-(((undecyloxy)carbonyl)amino)hexyl)-amino)hexyl)carbamate A46;
heptadecan-9-yl (6-((4-hydroxybutyl)(6-(((undecyloxy)carbonyl)amino)hexyl)-amino)hexyl)carbamate A47;
N,N-(((4-hydroxybutyl)azanediyl)bis(hexane-6,1-diyl))bis(2-hexyldecanamide) A48;
N,N-(((2-hydroxyethyl)azanediyl)bis(hexane-6,1-diyl))bis(2-hexyldecanamide) A49;
((((2-hydroxyethyl)azanediyl)bis(ethane-2,1-diyl))bis(oxy))bis(ethane-2,1-diyl) bis(2-hexyldecanoate) A50;
di(heptadecan-9-yl) 10-(2-hydroxyethyl)-4,7,13,16-tetraoxa-10-azanonadecane-dioate A51;
8,8′-((2-hydroxyethyl)azanediyl)bis(N-hexyl-N-octyloctanamide) A52;
heptadecan-9-yl 8-((2-hydroxyethyl)(3-oxo-4,7,10,13-tetraoxatetracosyl)amino)-octanoate A53; or
heptadecan-9-yl 8-((2-hydroxyethyl)(6-oxo-6-(2-(2-(pentyloxy)ethoxy)ethoxy)-hexyl)amino)octanoate A54;
or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, or hydrate thereof.

105. A compound of:
heptadecan-9-yl 6-((2-hydroxyethyl)(5-(nonyloxy)-5-oxopentyl)amino)hexanoate B1;
heptadecan-9-yl 6-((6-(heptyloxy)-6-oxohexyl)(2-hydroxyethyl)amino)hexanoate B2;
heptadecan-9-yl 6-((2-hydroxyethyl)(6-oxo-6-(undecyloxy)hexyl)amino)-hexanoate B3;
heptadecan-9-yl 7-((7-(heptyloxy)-7-oxoheptyl)(2-hydroxyethyl)amino)-heptanoate B4;
heptadecan-9-yl 7-((2-hydroxyethyl)(7-(nonyloxy)-7-oxoheptyl)amino)-heptanoate B5;
heptadecan-9-yl 7-((2-hydroxyethyl)(7-oxo-7-(undecyloxy)heptyl)amino)-heptanoate B6;
heptadecan-9-yl 8-((8-(heptyloxy)-8-oxooctyl)(2-hydroxyethyl)amino)octanoate B7;
heptadecan-9-yl 8-((2-hydroxyethyl)(8-oxo-8-(undecyloxy)octyl)amino)-octanoate B8;
heptadecan-9-yl 7-((2-hydroxyethyl)(6-oxo-6-(undecyloxy)hexyl)amino)-heptanoate B9;
heptadecan-9-yl 8-((6-(heptyloxy)-6-oxohexyl)(2-hydroxyethyl)amino)octanoate B10;
heptadecan-9-yl 8-((2-hydroxyethyl)(6-(nonyloxy)-6-oxohexyl)amino)octanoate B11;
heptyl 7-((6-(heptadecan-9-yloxy)-6-oxohexyl)(2-hydroxyethyl)amino)-heptanoate B12;
undecyl 7-((6-(heptadecan-9-yloxy)-6-oxohexyl)(2-hydroxyethyl)amino)-heptanoate B13;
heptyl 8-((6-(heptadecan-9-yloxy)-6-oxohexyl)(2-hydroxyethyl)amino)octanoate B14;
nonyl 8-((6-(heptadecan-9-yloxy)-6-oxohexyl)(2-hydroxyethyl)amino)octanoate B15;
undecyl 8-((6-(heptadecan-9-yloxy)-6-oxohexyl)(2-hydroxyethyl)amino)-octanoate B16;
nonyl 8-((7-(heptadecan-9-yloxy)-7-oxoheptyl)(2-hydroxyethyl)amino)octanoate B17;
heptadecan-9-yl 10-((2-hydroxyethyl)(8-(nonyloxy)-8-oxooctyl)amino)decanoate B18;
heptadecan-9-yl 10-((2-hydroxyethyl)(10-oxo-10-(undecyloxy)decyl)amino)-decanoate B19;
heptadecan-9-yl 6-((6-(heptyloxy)-6-oxohexyl)(3-hydroxypropyl)amino)-hexanoate B20;
heptadecan-9-yl 6-((3-hydroxypropyl)(6-(nonyloxy)-6-oxohexyl)amino)-hexanoate B21;
heptadecan-9-yl 7-((7-(heptyloxy)-7-oxoheptyl)(3-hydroxypropyl)amino)-heptanoate B22;
heptadecan-9-yl 8-((3-hydroxypropyl)(8-(nonyloxy)-8-oxooctyl)amino)octanoate B23;
heptadecan-9-yl 8-((3-hydroxypropyl)(8-oxo-8-(undecyloxy)octyl)amino)-octanoate B24;
heptadecan-9-yl 7-((3-hydroxypropyl)(6-(nonyloxy)-6-oxohexyl)amino)-heptanoate B25;
heptadecan-9-yl 8-((6-(heptyloxy)-6-oxohexyl)(3-hydroxypropyl)amino)-octanoate B26;
heptadecan-9-yl 8-((3-hydroxypropyl)(6-oxo-6-(undecyloxy)hexyl)amino)-octanoate B27;
di(heptadecan-9-yl) 6,6′-((2-hydroxyethyl)azanediyl)dihexanoate B28;
di(heptadecan-9-yl) 7,7′-((2-hydroxyethyl)azanediyl)diheptanoate B29;
((2-hydroxyethyl)azanediyl)bis(hexane-6,1-diyl) bis(2-hexyldecanoate) B30;
7-((2-hydroxyethyl)(8-oxo-8-(pentadecan-7-yloxy)octyl)amino)heptyl 2-hexyldecanoate B31;
((2-hydroxyethyl)azanediyl)bis(octane-8,1-diyl) bis(2-hexyldecanoate) B32;
((3-hydroxypropyl)azanediyl)bis(hexane-6,1-diyl) bis(2-hexyldecanoate) B33;
((3-hydroxypropyl)azanediyl)bis(heptane-7,1-diyl) bis(2-hexyldecanoate) B34;
((3-hydroxypropyl)azanediyl)bis(octane-8,1-diyl) bis(2-hexyldecanoate) B35;
((4-hydroxybutyl)azanediyl)bis(heptane-7,1-diyl) bis(2-hexyldecanoate) B36;
((4-hydroxybutyl)azanediyl)bis(octane-8,1-diyl) bis(2-hexyldecanoate) B37;
((5-hydroxypentyl)azanediyl)bis(hexane-6,1-diyl) bis(2-hexyldecanoate) B38;
((5-hydroxypentyl)azanediyl)bis(heptane-7,1-diyl) bis(2-hexyldecanoate) B39;
((5-hydroxypentyl)azanediyl)bis(octane-8,1-diyl) bis(2-hexyldecanoate) B40;
di(heptadecan-9-yl) 8,8′-((2-hydroxyethyl)azanediyl)dioctanoate B41;
di(heptadecan-9-yl) 8,8′-((4-hydroxybutyl)azanediyl)dioctanoate B42;
di(heptadecan-9-yl) 8,8′-((3-(dimethylamino)propyl)azanediyl)dioctanoate B43;
heptadecan-9-yl (Z)-8-((2-hydroxyethyl)(6-(non-2-en-1-yloxy)-6-oxohexyl)-amino)octanoate B44;
di(heptadecan-9-yl) 8,8′-((2-hydroxyethyl)azanediyl)dioctanoate B45;
((2-(2-methoxyethoxy)ethyl)azanediyl)bis(hexane-6,1-diyl) bis(2-hexyldecanoate) B46;
di(pentadecan-7-yl) 8,8′-((2-hydroxyethyl)azanediyl)dioctanoate B47; or
5-((2-hydroxyethyl)(7-((2-octyldecanoyl)oxy)heptyl)amino)pentyl dodecanoate B48;
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof.

106. A lipid nanoparticle comprising a compound of any one of claims 1 to 105.
